(19) United States
(12) Patent Application Publication
(10) Pub. No.: US 2017/0304261 A1
(43) Pub. Date: Oct. 26, 2017

(54) SILVESTROL, SILVESTROL ANALOGS AND USES THEREOF

(71) Applicant: Ohio State Innovation Foundation, Columbus, OH (US)

(72) Inventors: Robert A. Baiocchi, Dublin, OH (US); John Thomas Patton, Columbus, OH (US); David M. Lucas, Hilliard, OH (US); Douglas Kinghorn, Columbus, OH (US); Michael Grever, Powell, OH (US)

(21) Appl. No.: 15/372,904

(22) Filed: Dec. 8, 2016

Related U.S. Application Data
(63) Continuation of application No. 14/235,371, filed on May 25, 2014, now abandoned.
(60) Provisional application No. 61/512,135, filed on Jul. 27, 2011.

(51) Int. Cl. A61K 31/357; A61K 39/39; A61K 45/06; A61K 39/00

(52) U.S. Cl. CPC A61K 31/357; A61K 45/06; A61K 39/0011; A61K 39/39; A61K 2039/55511

(57) ABSTRACT

Disclosed herein are treatment methods, vaccination methods, compositions, co-therapeutic methods, combination therapeutic compositions and kits comprising silvestrol and silvestrol analogs alone or in combination with therapeutic and preventative therapies. Disclosed herein are also methods, compositions and kits that can be used to treat cancer, viral infections, to modulate the immune system or as preventative therapies.

### BACKGROUND

Cyclopenta[b]benzofuran constituents of plants from the genus Aglaia were first discovered in 1982, and have been of considerable interest since one such molecule, rocaglamide, was found to exhibit anti-leukemic activity in the P388 lymphocytic leukemic murine model [King 1982 Rose 1988]. The anti-proliferative activity of members of the cyclopenta[b]benzofuran class have been demonstrated using human tumor cell lines [Bohnenstengel 1999; Bohnenstengel 1999; Su 2006] (reviewed in Kim 2006) and primary human tumor cells [Zhu 2007]. Silvestrol, a member of this class, was identified from tropical plants by activity-guided chromatographic fractionation. The identification of cytotoxic extracts from Aglaia foveolata led to the purification of silvestrol as well as (+)-episilvestrol, its C-5′″S epimer at the diol side chain of the dioxanyl ring. Using detailed NMR studies and single-crystal X-ray diffraction, the structure and absolute configuration of silvestrol was then characterized [Hwang et al].

Silvestrol is a unique member of the cyclopenta[b]benzofuran class, and bears a bulky dioxanyl group unprecedented in nature. Preliminary structure-activity relationship studies indicate that the dioxanyl side chain is important for cytotoxicity, as silvestrol is much more potent than rocaglamide M vitro, and acetylation of the two dioxanyloxy hydroxyl groups causes a ten-fold reduction in potency [Hwang 2004]. Because of its unique structure and high potency, silvestrol has attracted the attention of synthetic organic chemists. The total syntheses of (−)-silvestrol has been demonstrated by El Sous 2007 and Gerard 2007, which confirmed the structure and stereochemistry reported earlier. The synthesis of silvestrol has been refined by Adams 2009, which refinement generated, additional bioactive derivatives [Adams 2009]. These studies indicate that stereochemistry of the cyclopentabenzofuran backbone was critical for the anti-cancer activity of silvestrol. Structure-activity relationship studies by Cencic 2009 confirmed the importance of both the cyclopenta[b]benzofuran core and side chain for optimal potency. While silvestrol was originally isolated from fruits and twigs of A. foveolata, work shows it can be successfully extracted from the leaves and stems of this plant [Salim 2007]. The occurrence of this compound in several plant parts including leaves enables silvestrol to be, produced in large quantities as a “renewable resource” that will not sacrifice the plant of origin.

### SUMMARY

Disclosed herein are treatment methods comprising the step or administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject. In an aspect, disclosed herein are treatment methods comprising the step of administering, to a subject diagnosed with a cancer, a composition comprising: (a) silvestrol or a silvestrol analog; (b) an and-cancer agent; and (c) a pharmaceutically acceptable carrier, wherein the composition is administered in a dosage and frequency synergistically effective to treat the cancer in the subject, wherein the anti-cancer agent is an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage, and frequency synergistically effective to treat the cancer in the subject, wherein the dosage of the anti-cancer agent is lower than the standard amount necessary to treat the cancer in the subject.

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the silvestrol analog is a compound, of Formula (I) or a salt or prodrug thereof or a compound (including stereoisomers within the dioxanyl group) of formula (i) or a salt or prodrug thereof.

In an aspect, disclosed herein are treatment methods comprising the step of administering to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the composition is administered in an amount in the range of 0.1 to 200 micrograms of complex per kg body weight of the subject per administration. In an aspect, disclosed herein are treatment methods comprising the step of administering, to a subject diagnosed with a cancer, a composition comprising; (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the silvestrol or a silvestrol analog of the composition is administered in an amount in the range of 0.1 to 0.3 micrograms of complex per kg body weight of the subject per administration. In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the silvestrol or a silvestrol analog of the composition is administered in an amount of 0.2 micrograms of complex per kg body weight of the subject per administration.

In an aspect, disclosed herein are treatment methods comprising the step of administering, to a subject diagnosed with a cancer, a composition comprising (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the composition is administered repeatedly to the subject. In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the subject is a mammal such as a human. In some aspects, the repeated administration can be daily, every other day, every third day, every forth day, every fifth day, every sixth day, or once per week. One of skill will be able to determine dosage regime based on the dosage of the compositions given to the subject and the subject him or herself.

In an aspect, disclosed herein are treatment methods comprising the step of administering, to a subject diagnosed with a cancer, a composition comprising: (a) silvestrol or a silvestrol analog; (b) an anti-cancer agent; and (C) a pharmaceutically acceptable carrier, wherein the composition is administered in a dosage and frequency synergistically effective treat the cancer in the subject, wherein the subject has been diagnosed with a need for modulating the immune system prior to the administering step.

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, further comprising the step of identifying a subject in need of modulating the immune system.

In an aspect, disclosed herein are treatment methods comprising the step of administering, to a subject diagnosed with a cancer, a composition comprising; (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the cancer is lymphoma, B cell lymphoma, T cell lymphoma, mycosis fungicides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein humoral immune function in the subject is unaffected, wherein Ig production in the subject is unaffected, wherein IFNg production in the subject remains normal, wherein the composition potentiates virus-specific or tumor-specific cytotoxic T lymphocytes, wherein the composition potentiates virus-specific or tumor-specific T-helper cells, wherein the composition potentiates virus-specific or tumor-specific T-helper cells, or wherein the composition potentiates virus-specific or tumor-specific Th1, Th2, Th17, or Treg cells.

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the composition further comprises one or more immunomodulatory agents.

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the one or more immunomodulatory agents is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein one or more immunomodulatory agents potentiates an antibody-dependent cell-mediated cytoxicity (“ADCC”) response in the subject.

Disclosed herein are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer. In an aspect, disclosed herein are vaccination methods comprising the step of administering, to a subject not yet diagnosed with a cancer, a composition comprising; (a) silvestrol or a silvestrol analog; (b) anti-cancer agent; and (c) a pharmaceutically acceptable carrier, wherein the composition is administered in a dosage and frequency synergistically effective to modulate the immune system in the subject, thereby vaccinating the subject against the cancer wherein the anti-cancer agent is an antibody, a preventive vaccine, a therapeutic vaccine, a cytokine, a biologic fusion construct, a nucleic acid constructs (i.e.: DNA-based vaccines or immune modulatory products), a receptor ligand, cytokine or receptor antagonist, or an adoptively transferred cell (NK, T cells, DCs).

In an aspect, are vaccination methods comprising the step of administering, to a subject not yet diagnosed with a cancer, a composition comprising: (a) silvestrol or a silvestrol analog; (b) anti-cancer agent; and (c) a pharmaceutically acceptable carrier, wherein the composition is administered in a dosage and frequency synergistically effective to modulate the immune system in the subject, thereby vaccinating the subject against the cancer, wherein the dosage of the anti-cancer agent is lower than the standard amount necessary to treat the cancer in the subject.

In an aspect, are vaccination methods comprising the step of administering, to a subject not yet diagnosed with a cancer, a composition comprising: (a) silvestrol or a silvestrol analog, (b) a anti-cancer agent; and (c) a pharmaceutically acceptable carrier, wherein the composition is administered in a dosage and frequency synergistically effective to modulate the immune system in the subject, thereby vaccinating the subject against the cancer, wherein the silvestrol analog is a compound of Formula (I) or a salt or prodrug thereof or a compound (including stereoisomers within the dioxanyl group) of formula (i) or a salt or prodrug thereof.

In an aspect, are vaccination methods comprising the step of administering, to a subject not yet diagnosed with a cancer, a composition comprising: (a) silvestrol or a silvestrol analog; (b) a anti-cancer agent, and (c) a pharmaceutically acceptable carrier, wherein the composition is administered in a dosage and frequency synergistically effective to modulate the immune system in the subject, thereby vaccinating the subject against the cancer wherein the composition is administered in an amount in the range of 0.1 to 200 micrograms of complex per kg body weight of the subject per administration. In an aspect, are vaccination methods comprising the step of administering, to a subject not yet diagnosed with a cancer, a composition comprising: (a) silvestrol or silvestrol analog; (b) anti-cancer agent; and (c) a pharmaceutically acceptable carrier, wherein the composition is administered in a dosage and frequency synergistically effective to modulate the immune system in the subject, thereby vaccinating the subject against the cancer wherein the silvestrol of silvestrol analog of the composition is administered in an amount in the range of 0.1 to 0.3 micrograms of complex per kg body weight of the subject per administration.

In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer wherein the composition is administered repeatedly to the subject. In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating, the subject against the caner wherein the subject is a mammal, such as a human.

In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer, wherein the subject has been diagnosed with a need for modulating the immune system prior to the administering step.

In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer, further comprising the step of identifying a subject in need of modulating the immune system. In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer, wherein humoral immune function in the subject is unaffected, wherein Ig production in the subject is unaffected, wherein IFNg production in the subject remains normal, wherein the composition potentiates virus-specific or tumor-specific cytotoxic T lymphocytes, wherein the composition potentiates virus-specific or tumor-specific T-helper cells, wherein the composition potentiates virus-specific or tumor-specific T-helper cells, or wherein the composition potentiates virus-specific or tumor-specific Th1, Th2, Th17, or Treg cells.

In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer, wherein the composition further comprises one or more immunomodulatory agents. In an aspect, the one or more immunomodulatory agents is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, are methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer, wherein the composition further comprises one or more immunomodulatory agents, wherein one or more immunomodulatory agents potentiates an antibody-dependent cell-mediated cytoxicity (“ADCC”) response in the subject.

Disclosed herein are compositions comprising (a) silvestrol or a silvestrol analog; (b) an anti-cancer agent; and (c) a pharmaceutically acceptable carrier. In an aspect, disclosed herein are compositions comprising (a) silvestrol or a silvestrol analog; (b) an anti-cancer agent; and (c) a pharmaceutically acceptable carrier, wherein the silvestrol or silvestrol analog is silvestrol, are compositions comprising (a) silvestrol or a silvestrol analog; (b) an anti-cancer agent; and (c) a pharmaceutically acceptable carrier, wherein the silvestrol or silvestrol analog is a silvestrol analog. In an aspect, disclosed herein are compositions comprising (a) silvestrol or a silvestrol analog; (b) an anti-cancer agent; and (c) a pharmaceutically acceptable carrier, wherein the silvestrol or silvestrol analog is silvestrol, wherein the silvestrol analog is a compound of Formula (I) or a salt or prodrug thereof or a compound (including stereoisomers within the dioxanyl group) of formula (i) or a salt or prodrug thereof.

In an aspect, disclosed herein are compositions comprising (a) silvestrol or a silvestrol analog; (b) an anti-cancer agent; and (c) pharmaceutically acceptable carrier, wherein the silvestrol or silvestrol analog is silvestrol, are compositions comprising (a) silvestrol or a silvestrol analog; (b) an anti-cancer agent; and (c) a pharmaceutically acceptable carrier, wherein the anti-cancer agent is an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

In an aspect, disclosed herein are compositions comprising (a) silvestrol or a silvestrol analog; (b) an anti-cancer agent; and (c) a pharmaceutically acceptable carrier, wherein the silvestrol or silvestrol analog is silvestrol, are compositions comprising (a) silvestrol or a silvestrol analog; (b) an anti-cancer agent; and (c) a pharmaceutically acceptable carrier, wherein the composition further comprises one or more immunomodulatory agents. In an aspect, disclosed herein are compositions comprising (a) silvestrol or a silvestrol analog; (b) an anti-cancer agent; and (c) a pharmaceutically acceptable carrier, wherein the silvestrol or silvestrol analog is silvestrol, are compositions comprising (a) silvestrol or a silvestrol analog; (b) an anti-cancer agent; and (c) a pharmaceutically acceptable carrier, wherein the composition further comprises one or more immunomodulatory agents, wherein the one or more immunomodulatory agents is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

Disclosed herein are methods comprising the step of administering a composition comprising silvestrol or silvestrol analog, and a pharmaceutically acceptable carrier, wherein the composition is administered in a dosage and frequency less than 0.5, 0.4, or 0.3 mg/kg/day. In an aspect, disclosed herein are methods comprising the step of administering a composition comprising silvestrol or a silvestrol analog, and a pharmaceutically acceptable carrier, wherein the silvestrol or silvestrol analog of the composition is administered in a dosage and frequency less than 0.5, 0.4, or 0.3 mg/kg/day.

In an aspect, disclosed herein are methods comprising the step of administering a composition comprising silvestrol or a silvestrol analog, and a pharmaceutically acceptable carrier, wherein the composition is administered in a dosage and frequency less than 0.5, 0.4, or 0.3 mg/kg/day, wherein the subject has been diagnosed with a cancer prior to administration. In an aspect, disclosed herein are methods comprising the step of administering a composition comprising silvestrol or a silvestrol analog, and a pharmaceutically acceptable carrier, wherein the silvestrol or silvestrol analog composition is administered in a dosage and frequency less than 0.5., 0.4, or 0.3 mg/kg/day, wherein the subject has been diagnose with a cancer prior to administration.

In an aspect, disclosed herein are methods comprising the step of administering a composition comprising silvestrol or a silvestrol analog, and a pharmaceutically acceptable carrier, wherein the composition is administered in a dosage and frequency less than 0.5, 0.4, or 0.3 mg/kg/day, wherein the silvestrol analog is a compound of Formula (I) or a salt or prodrug thereof or a compound (including stereoisomers within the dioxanyl group) of formula (i) or a salt prodrug thereof. In an aspect, disclosed herein are methods comprising the step of administering a composition comprising silvestrol or a silvestrol analog, and a pharmaceutically acceptable carrier, wherein the silvestrol analog of the composition is administered in a dosage and frequency less than 0.5, 0.4, or 0.3 mg/kg/day, wherein the silvestrol analog is a compound of Formula (I) or a salt or prodrug thereof or a compound (including stereoisomers within the dioxanyl group) of formula (i) or a salt or prodrug thereof.

In an aspect, disclosed herein are methods comprising the step of administering a composition comprising silvestrol or a silvestrol analog, and a pharmaceutically acceptable carrier, wherein the compositions administered in a dosage and frequency less than 0.5, 0.4, or 0.3 mg/kg/day, wherein the composition further comprises one or more immunomodulatory agents. In an aspect, disclosed herein are methods comprising the step of administering a composition comprising silvestrol or a silvestrol analog, and a pharmaceutically acceptable carrier, wherein the silvestrol or silvestrol analog of the composition is administered in a dosage and frequency less than 0.5, 0.4, or 0.3 mg/kg/day, wherein the composition further comprises one or more immunomodulatory agents.

In an aspect, disclosed herein are methods comprising the step of administering a composition comprising silvestrol or a silvestrol analog, and a pharmaceutically acceptable carrier, wherein the composition is administered in a dosage and frequency less than 0.5, 0.4, or 0.3 mg/kg/day, wherein the composition further comprises one or more immunomodulatory agents, wherein the one or more immunomodulatory agents is a cytokine, immune system adjuvant, fusion protein, an antibody, a preventive vaccine, a therapeutic vaccine, a cytokine, biologic fusion construct, a nucleic acid constructs (i.e.: DNA-based vaccines or immune modulatory products), a receptor ligand, cytokine or receptor antagonist, or an adoptively transferred cell (NK, T cells, DCs). In an aspect, disclosed herein are methods comprising the step of administering a composition comprising silvestrol or a silvestrol analog, and a pharmaceutically acceptable carrier, wherein the silvestrol or silvestrol analog composition is administered in a dosage and frequency less than 0.5, 0.4, or 0.3 mg/kg/day, wherein the composition further comprises one or more immunomodulatory agents, wherein the one or immunomodulatory agents is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

In an aspect, disclosed herein are methods comprising the step of administering a composition comprising silvestrol or a silvestrol analog, and a pharmaceutically acceptable carrier, wherein the composition is administered in a dosage and frequency less than 0.5, 0.4, or 0.3 mg/kg/day, wherein the composition further comprises one or more immunomodulatory agents, wherein one or more immunomodulatory agents potentiates an antibody-dependent cell-mediated cytoxicity (“ADCC”) response in the subject. In an aspect, disclosed herein are methods comprising the step of administering a composition comprising silvestrol or a silvestrol analog, and a pharmaceutically acceptable carrier, wherein the silvestrol or silvestrol analog of the composition is administered in a dosage and frequency less than 0.5, 0.4, or 0.3 mg/kg/day, wherein the composition further comprises one or more immunomodulatory agents, wherein one or more immunomodulatory agents potentiates an antibody-dependent cell-mediated cytoxicity (“ADCC”) response in the subject.

Disclosed herein are methods comprising the step of administering, to a subject not yet diagnosed with a cancer, a composition comprising silvestrol or a silvestrol analog, and a pharmaceutically acceptable carrier, wherein the composition is administered in a dosage and frequency effective to modulate the immune system in the subject. In an aspect, disclosed herein are methods comprising the step of administering, to a subject not yet diagnosed with a cancer, a composition comprising silvestrol or a silvestrol analog, and a pharmaceutically acceptable carrier, wherein the composition administered in a dosage and frequency effective to modulate the immune system in the subject, wherein the dosage is less than 0.5, 0.4, or 0.3 mg/kg/day. In an aspect, disclosed herein are methods comprising the step of administering, to a subject not yet diagnosed with a cancer, a composition comprising silvestrol or a silvestrol analog, and a pharmaceutically acceptable carrier, wherein the composition is administered in a dosage and frequency effective to modulate the immune system in the subject. In an aspect, disclosed herein are methods comprising the step of administering, to a subject not yet diagnosed with a cancer, a composition comprising silvestrol or a silvestrol analog, and a pharmaceutically acceptable carrier, wherein the silvestrol or silvestrol analog of the composition is administered in a dosage and frequency effective to modulate the immune system in the subject, wherein the dosage is less than 0.5, 0.4, or 0.3 mg/kg/day. In some aspects, the silvestrol or silvestrol analog of the composition is administered in a dosage and frequency effective to modulate the immune system in the subject, wherein the dosage is 0.2 mg/kg/day.

In an aspect, disclosed herein are methods comprising the step of administering, to a subject not yet diagnosed with cancer, a composition comprising silvestrol or silvestrol analog, and a pharmaceutically acceptable carrier, wherein the composition is administered in a dosage and frequency effective to modulate the immune system in the subject, wherein the silvestrol or silvestrol analog is silvestrol.

In an aspect, disclosed herein are methods comprising the step of administering, to a subject not yet diagnosed with a cancer, a composition comprising silvestrol or a silvestrol analog, and a pharmaceutically acceptable carrier, wherein the composition is administered in a dosage and frequency effective to modulate the immune system in the subject, wherein the silvestrol or silvestrol analog is a silvestrol analog.

In an aspect, disclosed herein are methods comprising the step of administering, to a subject not yet diagnosed with a cancer, a composition comprising silvestrol or a silvestrol analog, and a pharmaceutically acceptable carrier, wherein the composition is administered in a dosage and frequency effective to modulate the immune system in the subject, wherein the composition further comprises one or more immunomodulatory agents.

Disclosed herein are compositions comprising silvestrol or a silvestrol analog, and a pharmaceutically acceptable carrier, wherein the composition is provided in a dosage and frequency less than 0.5 mg/kg/day but effective to modulate the immune system in a subject.

In an aspect, disclosed herein are compositions comprising silvestrol or a silvestrol analog, and a pharmaceutically acceptable carrier, wherein the composition is provided in a dosage and frequency less than 0.5 mg/kg/day but effective to modulate the immune system in a subject wherein the composition further comprises one or more immunomodulatory agents.

Disclosed herein are co-therapeutic methods comprising administration of silvestrol or a silvestrol analog, and an agent having a side-effect of immunosuppression.

Disclosed herein are co-therapeutic methods comprising administration of silvestrol or a silvestrol analog, and an anti-viral agent. In an aspect, disclosed herein are co-therapeutic methods comprising administration of silvestrol or a silvestrol analog and an anti-viral agent, wherein the dosage of the anti-viral agent is lower than the standard amount necessary to treat the viral infection in the subject.

Disclosed herein are combination therapeutic compositions comprising silvestrol or a silvestrol analog, and an anti-viral agent.

Disclosed herein are kits comprising at least silvestrol or a silvestrol analog or a pharmaceutically acceptable salt thereof, and one or more of: an anti-cancer agent, an immune modulatory agent, an agent having a side-effect of immunosuppression, an anti-viral agent, an agent known to modulate the immune system; at least one agent known to modulate the activity of an immune cell; at least one agent known to modulate viral gene expression; at least one agent known to treat cancer; at least one, agent known to treat one or more symptoms of cancer; at least one agent known to treat a disease state or conditions associated with cellular hyperproliferation; instructions for modulating the immune system in a subject; instructions for modulating the activity of an immune cell in a subject; instructions for treating a disease state or conditions associated with cellular hyperproliferation; instructions for treating a cancer in a subject, instructions for vaccinating a subject not yet diagnosed with cancer, instructions for administering a composition comprising silvestrol or a silvestrol analog and a pharmaceutically acceptable carrier to a subject, instructions for administration of silvestrol or a silvestrol analog and an agent having a side-effect of immunosuppression to a subject, or instructions for administration of silvestrol or a silvestrol analog and an anti-viral agent to a subject.

While aspects of the present invention can be described and claimed in a particular statutory class, such as the system statutory class, this is for convenience only and one of skill in the art will understand that each aspect of the present invention can be described and claimed in any statutory class.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying Figures, which are incorporated in and constitute a part of this specification, illustrate several aspects and together with the description serve to explain the principles of the invention.

FIG. 1 shows a schematic for the production of EBV⁺ LCLs derived from SCID mice.

FIG. 2 shows level of (A) apoptosis (represented as relative % viability) and (B) proliferation (represented as relative % growth) of LCLs following exposure to silvestrol.

FIG. 3 shows immunoblots following exposure to silvestrol for (A) LMP-1 expression in seven different LCLs, (B) LMP-1 expression in a single LCL on days 1, 3, and 5, (C) LMP-1 expression and expression of other EBV proteins, and (D) STAT and Akt expression on days 1, 3, and 5.

FIG. 4 shows a schematic detailing the interaction between LMP-1 CTAR domains and downstream pathways such as NFκB and Akt pathways.

FIG. 5 shows immunoblots following exposure to silvestrol for expression of proteins including LMP-1, NF-κB, and Akt.

FIG. 6 shows immunoblots for expression of various proteins in the STAT and Akt pathways following silvestrol exposure.

FIG. 7 shows (A) immunoblots of LMP-1 expression in six different LCLs following exposure to silvestrol and (B) the relative viability of the LCLs in (A).

FIG. 8 shows the viability relative to control plotted against relative LMP-1 levels (untreated), which correlation generated an R² value of 0.2984.

FIG. 9 shows data related to the expansion of cytotoxic T-lymphocytes (CD3₊/CD8₊) (CTLs) following exposure to various concentrations of silvestrol. (A) shows relative population of CTLs and (B) shows the relative cell number of CTLs.

FIG. 10 shows data related to the expansion of helper T-cells (CD3₊/CD4₊) following exposure to various concentrations of silvestrol. (A) shows relative population of helper T-cells and (B) shows the relative cell number of helper T-cells.

FIG. 11 shows data related to the expansion of NK cells (CD3₋/CD56₊) following, exposure to various concentrations of silvestrol. (A) shows relative population of NK cells and (B) shows the relative cell number of NK cells.

FIG. 12 shows data related to the expansion of co-cultures of non-irradiated DC9 cells following exposure to various concentration of silvestrol. (A) shows the % of viable population of NK cells (CD3⁻/CD56³⁰ ) and (B) shows the number of viable expanded NK cells (CD3⁻/CD56³⁰ ) cells.

FIG. 13 shows data related to the expansion of co-cultures of non-irradiated DC9 cells following exposure to various concentrations of silvestrol. (A) shows the % of viable population of cytotoxic T-lymphocytes (CD3⁺/CD8⁺) and (B) shows the number of viable expanded cytotoxic T-lymphocytes (CD3⁺/CD8⁺).

FIG. 14 shows data related to the expansion of co-cultures of non-irradiated MAC cells following exposure to various concentrations of silvestrol. (A) shows the % of viable population of NK cells (CD3⁻/CD56³⁰ ) and (B) shows the number of viable expanded NK cells (CD3⁻/CD56³⁰ ) cells.

FIG. 15 shows data related to the expansion of co-cultures of non-irradiated MAC cells following exposure to various concentrations of silvestrol. (A) shows the % of viable population of CTLs (CD3⁺/CD8⁺) and (B) shows the number of viable expanded CTLs (CD3⁺/CD8⁺).

FIG. 16 shows the effect of exposure to silvestrol at various concentrations on LCLs only, on PBMCs, and on non-irradiated co-cultures.

FIG. 17 shows % toxicity of immune effector subsets as assessed via CFSE⁺/7-AAD⁺ staining following exposure to silvestrol at various concentrations.

FIG. 18 shows IFNgamma production of similar immune effector subsets when plated against irradiated co-cultures of DC9 cells.

FIG. 19 shows the relative cytotoxicity on DC9 cells following pretreatment with silvestrol and subsequent treatment with various agents including rituximab and herceptin.

FIG. 20 shows the relative cytotoxicity of NK cells from co-cultures following treatment with rituximab and herceptin.

FIG. 21 shows data regarding the expansion of NK cells following silvestrol treatment.

FIG. 22 shows the relative cytotoxicity of various cells including MAC cells and DC9 cells and co-cultures following treatment with rituximab and herceptin.

FIG. 23 shows data regarding engraftment of human PBMCs following exposure to silvestrol as measured by the production of human immunoglobulin.

FIG. 24 shows data regarding the body weight of mice following exposure to silvestrol.

FIG. 25 shows (A) data regarding spleen size of mice following exposure to silvestrol and (B) representative images of spleens.

FIG. 26 shows a Kaplan-Meier analysis of overall survival in mice exposed to silvestrol.

FIG. 27 shows a schematic for evaluating the effect of exposure to silvestrol on immune surveillance.

FIG. 28 shows data related to the expansion of cytotoxic T-lymphocytes (CTLs) (CD3⁺/CD8⁺) and helper T-cells (CD3⁺/CD4⁺) following exposure to various concentrations of silvestrol. (A) shows relative population of CD3⁺/CD8⁺ CTLs and (B) shows the relative population of CD3⁺/CD4⁺helper T-cells. Co-cultures (CoCx) were created by mixing irradiated LCL with equal numbers of autologous PBMC. CoCx were incubated in the presence of 10 U/ml IL-2 and given a single dose of 0 (vehicle only), 2, 5, or 10 nM silvestrol. Flow cytometric analysis was conducted on day 14. Cells were gated on Live events expressed as percentage viable population, relative to the vehicle CoCx condition, for (A) CD3⁺/C8⁺; (B) CD3⁺/CD4⁺

FIG. 29 shows data related to the expansion of NK cells (CD3⁺/CD56⁺) following exposure to various concentrations of silvestrol. Data are presented as relative population of CD3⁻/CD56⁺ NK cells. CO-cultures (CoCx) were created by mixing irradiated LCL with equal numbers of autologous PBMC, CoCx were incubated in the presence of 10 U/ml IL-2 and given a single dose of 0 (vehicle only), 2, 5, or 10 nM silvestrol. Flow cytometric analysis was conducted on day 14. Cells were gated on live events and analyzed for percentage of viable populations of CD3⁻/CD56⁺, relative to the vehicle CoCx condition. Results shown are the averages from three individual CoCx.

FIG. 30 shows data related to the expansion of CD3⁺/CD8⁺ CTLs and CD3⁻/CD56⁺ NK cells following exposure to various concentration of silvestrol. (A) shows the % of viable population of CD3⁺/CD8⁺ CTLs and (B) shows the % of viable population of CD3⁻/CD56⁺ NK cells. CoCx were created by mixing non-irradiated LCL with equal numbers of autologous PBMC. CoCx were incubated in the presence of 10 U/ml IL-2 with or without silvestrol and flow cytometric analysis was conducted on day 10. Live events were gathered by gating on cells negative for LIVE/DEAD stain. Data are expressed as percentage of total viable population for (A) CD3⁺/CD8⁺; (B) CD3⁻/C56⁺.

FIG. 31 shows % toxicity of cells as assessed via CFSE⁺/7-AAD₊ staining following exposure to silvestrol at various concentrations.

FIG. 32 shows % toxicity of cells as assessed via CFSE⁺/7-AAD₊ staining foilowing various treatment protocols including co-culturing.

FIG. 33 shows data regarding the immunomodulatory activity in irradiated LCL co-cultures following exposure to silvestrol. Relative to the vehicle co-culture condition, the relative % population is shown for (A) total cells, (B) CD3⁺/CD4⁺ helper T cells, (C) CD3⁺/CD8⁺ CTLs, and (D) CD3⁻/CD56⁺NK cells.

FIG. 34 shows data regarding the immunomodulatory activity in non-irradiated LCL co-cultures following exposure to silvestrol. FIG. 34 shows the % population for (A) CD3⁻/CD19⁺ LCLs, (B) CD3⁺/CD4⁺ helper T cells. (C) CD3⁺/CD8⁺ CTLs, and (D) CD3⁻/CD56⁺ NK cells. (E) shows that when left untreated, the CD3⁻/CD19⁺ LCLs proliferated and matched the expansion of the effector subsets.

FIG. 35 shows data regarding the effect that exposure to silvestrol has on adaptive and innate immune effectors. FIG. 35 shows the relative cytotoxicity of (A) expanded effector cells activity against autologous LCLs, (B) effectors exposed to silvestrol, (C) autologous NK cells following exposure to rituximab, and (D) s non-autologous NK cells following exposure to rituximab.

FIG. 36 shows immunoblots of various proteins following exposure to silvestrol including NF-κB proteins (left panel) and apoptotic and epigenetic proteins (right panel).

FIG. 37 shows data regarding the immunomodulatory activity in non-irradiated LCL co-cultures following exposure to silvestrol for two additional donors.

FIG. 38 shows relative INFgamma release from various cells (CTLs, NK cells, and helper T-cells) following exposure to silvestrol during (A) non-specific stimulation (TPA/INO) and (B) specific stimulation (autologous LCLs). Co-cultures (CoCx) were created by mixing irradiated LCL with equal numbers of autologous PBMC. CoCx were incubated in the presence of 10 U/ml IL-2 and given a single dose of 0 (vehicle only), 2, 5, or 10 nM silvestrol. At day 14 cells were (A) stimulated non-specifically by addition of 12-O-tetradocanoylphorbol-13-acetate (TPA) and ionomycin (INO) to assay for the ability to release interferon gamma (IFNγ) or (B) by the addition of equal numbers autologous LCL to measure cell mediated response to antigen-induced IFNγ production. IFNγ production was measured by intercellular flow following four hour stimulation.

FIG. 39 shows data regarding expansion of co-cultures of DC9 cells following alteration of an effector cell population and (A) exposure to silvestrol at varying concentrations or (B) exposure to silvestrol at a singular concentration. CoCx were created by mixing non-irradiated LCL with equal numbers of autologous PBMC that had been (1) non-depleted; (2) CD8 depleted; (3) CD56 depleted; (4) CD3 depleted; (5) T cell enriched by magnetic bead separation. CoCx were incubated in the presence of 10 U/ml IL-2 with or without silvestrol and flow cytometric analysis was conducted on day 10 by assaying for viable B cell (CD3−/CD19+) population.

FIG. 40 shows data regarding % of viable population of co-cultures of DC9 cells following exposure to silvestrol and (A) RAK tetramer and (B) FLR tetramer. Co-cultures (CoCx) were excited by mixing irradiated LCL with equal numbers of autologous PBMC. CoCx were incubated in the presence of 10 U/ml IL-2 and given a single dose of 0 (vehicle only), 2, 5, or 10 nM silvestrol. At day 14 flow cytometric analysis was conducted to assay for cytotoxic T cell population specific for EBV viral proteins. Cells were incubated with class 1 MHC tetramers bound to (A) BZLF-1 peptide (RAK) or (B) EBNA-3A (FLR) and gated on CD3+/CD8+ cells bound to tetramer.

FIG. 41 shows data regarding the NK cytotoxicity of K562 cells.

FIG. 42 shows data regarding the cytotoxicity of LCLs pretreated with BZLF1.

FIG. 43 shows data relating to CD3⁺/CD4⁺ helper T cells IFNgamma release in co-cultures of LCLs pretreated with BZLF1.

FIG. 44 shows data relating to CD3⁺/CD8⁺ CTLs IFNgamma release in co-cultures of LCLs pretreated with BZLF1.

FIG. 45 shows data relating to induction of IL-6 exposure to silvestrol.

FIG. 46 shows data regarding LMP-3 expression following exposure to silvestrol.

FIG. 47 shows data regarding EBNA1 expression following exposure to silvestrol.

FIG. 48 shows a schematic of a translation initiation complex with inhibitors including silvestrol.

FIG. 49 shows data relating to T cell and NK cell activation markers and total T cell populations for co-cultures and PBMCs exposed to silvestrol and for co-cultures and PBMCs not exposed to silvestrol. CoCx were created by mixing non-irradiated LCL with equal numbers of autologous PBMC. CoCx or PBMC alone were incubated in the presence of 10 U/ml IL-2 with or without 10 nM silvestrol and flow cytometric analysis was conducted on day 10.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned, herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

a. Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, EIZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl, ” or “a residue” includes mixtures of two or more such functional groups, alkyls, or residues, and the like.

The word “or” as used herein means any one member of a particular list and also includes any combination members of that list.

As used herein, “modulate”, “modulating”, or “modulated” means to alter, by increasing or decreasing.

As used herein, “unaffected” refers to the lack of a change prior to a specific action.

By “normal subject” is meant an individual who does not have a disease or symptom of a disease. For example, in the context of cancer, a “normal” subject is a subject without cancer or symptoms of cancer. As another example, a “normal” subject” in the context of viral infections, would be a subject without a viral infection or symptom of viral infection.

As used herein, “anti-cancer agent” refers to an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs) that when administered to a subject with cancer treats or prevents cancer in the subject. As used herein, “anti-cancer agent” also refers to a composition that reduces or prevents a cancerous tissue from forming or growth of a cancerous tissue.

As used herein, a “vaccine” refers to compositions that boost the immune system's natural ability to protect the body against “foreign invaders,” mainly infectious agents, that may cause disease. For example, vaccines can prevent cancer from developing in healthy people or can treat an existing cancer by strengthening the body's natural defenses against the cancer. Such vaccines can be referred to as preventative (or prophylactic) vaccines and treatment or therapeutic vaccines, respectively. Examples of preventative or therapeutic vaccines include, but are not limited to tumor cell vaccines, antigen vaccines, dendritic cell vaccines, DNA vaccines, and vector-based vaccines.

As used herein, a “tumor cell vaccine” refers to vaccines made from actual cancer cells that have been removed from a subject. The cells are treated in the lab, usually with radiation, so they cannot form more tumors. In some cases, doctors also change the cells in certain ways, often by adding chemicals or new genes, to make them more likely to be seen as foreign by the immune system. The cells can then be injected into the subject. Tumor cell vaccines can be autologous, meaning the vaccine is made from killed tumor cells taken from the same subject in whom they will later be used. Tumor cell vaccines can also be allogeneic, meaning the cells for the vaccine come from someone other than the patient being treated.

As used herein, an “antigen vaccine” refers to vaccines that boosts the immune system by using only one antigen (or a few), rather than whole tumor cells that contain many thousands of antigens. The antigens can be polypeptides. Antigen vaccines can be specific for a certain type of cancer. Antigen vaccines can also be comprised of a mixture of more than one antigen.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

In addition, as used herein, the term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B.C. Johnson, Ed., Academic Press, New York, pp, 1-12, (1983)).

As used herein, the term “amino acid sequence” refers to a list of abbreviations, letters, characters or words representing amino acid residues.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

As used herein, a “dendritic cell vaccine” refers to autologous vaccines and are often made individually for each subject. An example of a dendritic cell vaccine is Sipuleucel-T (Provenge), which is used to treat advanced prostate cancer.

As used herein, a “DNA vaccine” refers to vaccines composed of nucleic acids.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which incorporated into a vector, such as an autonomously replicating plasmid or virus; or incorporated into the genomic DNA of a prokaryote or eukaryote (e.g., transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

As used herein, a “Vector-based vaccine” refers to viruses, bacteria, yeast cells, or other structures that can be used to get antigens or DNA into the body. As used herein, a “Vector-based vaccine” refers to a composition that comprises a vector.

A “Vector” refers to a vehicle used to transfer genetic material to a target cell. Examples of vectors include, but are not limited to expression plasmids vectors, and viral vectors.

Expression vectors can be any nucleotide construction used to deliver genes into cells (e.g., a plasmid), or as part of a general strategy to deliver genes, e.g., as part of recombinant retrovirus or adenoviros (Ram et al. Cancer Res. 53:83-88, (1993)). The expression vectors can include a nucleic acid sequence encoding an anti-cancer agent, an anti-viral agent, or an immunomodulatory agent.

As used herein, plasmid or viral vectors are agents that transport the a nucleic acid, into a cell without degradation and include a promoter yielding expression of the gene in the cells into which it is delivered. In some embodiments the isolated polynucleotides disclosed herein are derived from either a virus or a retrovirus.

Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Marine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason, are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i,e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gent transfer. In Microbiology-1985, American Society for Microbiology, pp, 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery, provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:2S129-2S134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol, 51:650-655 (1984); Seth, et al., Mol. Cell. Biol. 4:1528-1533 (1984); Varga et al., J. Virology 65:6061-6070(1931); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The disclosed vectors thus can provide DNA molecules that are capable of integration into a mammalian chromosome without substantial toxicity.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

In some aspects, the vaccine is a Anthrax Vaccine, BCG Live, Diphtheria & Tetanus Toxoids Adsorbed, Diphtheria & Tetanus Toxoids, Adsorbed Diphtheria & Tetanus, Toxoids & Acellular Pertussis Vaccine, Diphtheria & Tetanus Toxoids & Acellular Pertussis Vaccine, Diphtheria & Tetanus Toxoids & Acellular Pertussis Vaccine, Diphtheria & Tetanus Toxoids & Acellular Pertussis Vaccine, Hepatitis B (recombinant) and Inactivated Poliovirus Vaccine Combined, Diphtheria and Tetanus Toxoids and Acellular Pertussis Adsorbed and Inactivated Poliovirus Vaccine, Diphtheria and Tetanus Toxoids and Acellular Pertussis, Inactivated Poliovirus and Haemophilus b Conjugate (Tetanus Toxoid Conjugate) Vaccine Haemophilus b Conjugate Vaccine, (Meningococcal Protein Conjugate), Haemophilus b Conjugate Vaccine (Tetanus Toxoid Conjugate), Haemophilus b Conjugate Vaccine (Tetanus Toxoid Conjugate), Haemophilus b Conjugate Vaccine (Meningococcal Protein Conjugate) & Hepatitis B Vaccine (Recombinant), Hepatitis A Vaccine, Inactivated, Hepatitis A Vaccine, Inactivated, Hepatitis A Inactivated and Hepatitis B (Recombinant) Vaccine, Hepatitis B Vaccine (Recombinant), Hepatitis B Vaccine (Recombinant), Human Papillomavirus Quadrivalent (Types 6, 11, 16, 18) Vaccine, Recombinant, Human Papillomavirus Bivalent (Types 16, 18) Vaccine, Recombinant, Influenza A (H1N1) 2009 Monovalent Vaccine, Influenza A (H1N1) 2009 Monovalent Vaccine, Influenza A (H1N1) 2009 Monovalent Vaccine, Influenza A (H1N1) 2009 Monovalent Vaccine, Influenza A (H1N1) 2009 Monovalent Vaccine, Influenza Virus Vaccine, H5N128 (for National Stockpile) Influenza Virus Vaccine (Trivalent, Types A and B), Influenza Virus Vaccine (Trivalent, Types A and B), Influenza Vaccine, Live, Intranasal (Trivalent Types A and B), Influenza Virus Vaccine (Trivalent, Types A and B), Influenza Virus Vaccine (Trivalent Types A and B), Influenza Virus Vaccine (Trivalent, Types A and B), Influenza Virus Vaccine (Trivalent, Types A and B), Influenza Vaccine,Live, Intranasal (Quadrivalent, Types A and Types B), Japanese Encephalitis Virus Vaccine, Inactivated, Adsorbed, Japanese Encephalitis Virus Vaccine Inactivated, Measles Virus Vaccine, Live, Measles and Mumps Virus Vaccine, Live Measles, Mumps, and Rubella Virus Vaccine, Live, Measles, Mumps, Rubella and Varicella Virus Vaccine Live, Meningococcal (Groups A, C, Y, and W-135) Oligosaccharide Diphtheria CRM197 Conjugate Vaccine, Meningococcal Groups C and Y and Haemophilus b Tetanus Toxoid Conjugate Vaccine, Meningococcal Polysaccharide (Serogroups A, C, Y and W-135) Diphtheria Toxoid Conjugate Vaccine, Meningococcal Polysaccharide Vaccine, Groups A, C. Y and W-135 Combined, Mumps Virus Vaccine Live Plague Vaccine Pneumococcal Vaccine, Polyvalent, Pneumococcal 7-valent Conjugate Vaccine, (Diphtheria CRM197 Protein) Pneumococcal 13-valent Conjugate Vaccine, (Diphtheria CRM197 Protein) Poliovirus Vaccine Inactivated (Human Diploid Cell) Poliovirus Vaccine Inactivated (Monkey Kidney Cell), Rabies Vaccine, Rabies Vaccine, Rabies Vaccine Adsorbed Rotavirus Vaccine, Live, Oral, Rotavirus Vaccine, Live, Oral, Pentavalent, Rubella Virus Vaccine Live, Smallpox (Vaccinia) Vaccine, Live, Tetanus & Diphtheria Toxoids Adsorbed for Adult Use, Tetanus & Diphtheria Toxoids Adsorbed for Adult Use, Tetanus & Diphtheria Toxoids Adsorbed for Adult Use Tetanus Toxoid, Tetanus Toxoid Adsorbed, Tetanus Toxoid, Reduced Diphtheria Toxoid and Acellular Pertussis Vaccine, Adsorbed, Tetanus Toxoid, Reduced Diphtheria Toxoid and Acellular Pertussis Vaccine, Adsorbed, Typhoid Vaccine Live Oral Ty21a, Typhoid Vi Polysaccharide Vaccine, Varicella Virus Vaccine Live, Yellow Fever Vaccine, or Zoster Vaccine, Live, (Oka/Merck) vaccines.

Other useful systems include, for example, plicating and host-restricted non-replicating vaccinia virus vectors.

By “sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

A “subject with cancer”, “cancer subject”, or a “subject diagnosed with cancer” is a subject with cancerous tissue.

As used herein, “cancerous tissue” is meant to mean a tissue that comprises neoplastic cells, exhibits an abnormal growth of cells and/or hyperproliferative cells. As used herein, the term “neoplastic” means an abnormal growth of a cell or tissue (e.g., a tumor or non-solid hyper proliferative cellular activity) which may be benign or cancerous. As used herein, “abnormal growth of cells” and/or “hyperproliferative cells” are meant to refer to cell growth independent of normal regulatory mechanisms (e.g., loss of contact inhibition), including the abnormal growth of benign and malignant cells or other neoplastic diseases. As used herein, the term “tumor” includes neoplasms that are identifiable through clinical screening or diagnostic procedures including, but not limited to, palpation, biopsy, cell proliferation index, endoscopy, mammography, digital mammography, ultrasonography, computed tomography (CT), magnetic resonance imaging (MRI), positron emission tomography (PET), radiography, radionuclide evaluation, CT- or MRI-guided aspiration cytology, and imaging-guided needle biopsy, among others. Such diagnostic techniques are well known to those skilled in the art and are described in Holland, et al., Cancer Medicine, 4th Ed., Vol. One, Williams & Wilkins, Baltimore, Md. (1997).

As used herein, “immunomodulatory agents” refers to compositions that have an effect on the immune system. For example, an immunomodulatory agent can be an immunosuppressant, immunostimulant, or tolerogen. Immunosuppressants inhibit immune response. Immunostimulants increase the immune response. Tolerogens induce tolerance and make a tissue non-responsive to antigen. Non-limiting examples of immunomodulatory agents includes, but are not limited to, a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

As used herein, “potentiate”, “potentiates”, “potentiated” refers to increasing the effectiveness of or making something more potent or powerful.

As used herein, an “antibody-dependent cell-mediated cytoxicity (“ADCC”) response” refers to a mechanism of cell-mediated immune defense whereby an effector cell of the immune system actively lyses a target cell that has been bound by specific antibodies. Classical ADCC is mediated by natural killer (NK) cells; neutrophils and eosinophils can also mediate ADCC.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, “silvestrol” refers to a composition having the following structure:

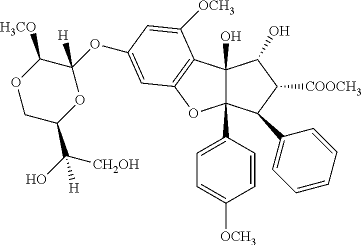 [SMILES: COC(=O)[C@H]1[C@@H](O)[C@@]2(O)c3c(OC)cc(O[C@@H]4O[C@@H]([C@H](O)CO)CO[C@H]4OC)cc3O[C@@]2(c2ccc(OC)cc2)[C@@H]1c1ccccc1]

Silvestrol can be isolated from the fruits and twigs of Aglaia foveolata or chemically synthesized using methods known in the art.

As used herein, the term “analog” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. For example, a “silvestrol analog” refers to a compound having a structure derived from the structure of silvestrol and whose structure is sufficiently similar to silvestrol and based upon that similarity, would be expected by one skilled in the art to exhibit the same or similar activities and utilities as silvestrol, or to induce, as a precursor, the same or similar activities and utilities as silvestrol. Examples of silvestrol analogs include, but are not limited to, compounds of Formula (I) or a salt or prodrug thereof.

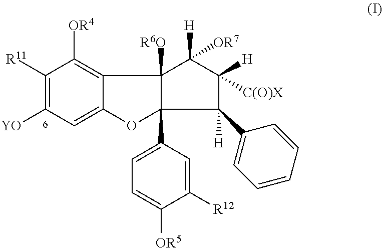

wherein
each R⁴-R¹⁰ is independently selected from the group consisting of hydrogen, optionally substituted alkyl, optionally substituted acyl, optionally substituted aryl, optionally substituted arylalkyl, optionally substituted cycloalkylalkyl, optionally substituted arylacyl, optionally substituted cycloalkylacyl and a C-1 linked saccharide;
X is OR⁸ or NR⁹ R¹⁰;
R¹¹ and R¹² are preferably each independently hydrogen or, alternatively, OR⁴ and R¹¹, and/or OR⁵ and R¹² together form a methylenedioxy group; and
Y is selected from the group consisting of optionally substituted phenyl, optionally substituted benzyl, optionally substituted benzoyl, optionally substituted C₃-C₈ cycloalkyl, (preferably optionally substituted C₅-C₆ cycloalkyl) optionally substituted CH₂—(C₃-C₈ cycloalkyl) (preferably optionally substituted CH₂—(C₅-C₆ cycloalkyl ), optionally substituted 5-6 membered heterocyclyl, and optionally substituted CH₂-(5-6 membered heterocyclyl).

Examples of silvestrol analogs also include, but are not limited to, compounds (including stereoisomers within the dioxanyl group) of formula (i) or a salt or prodrug thereof.

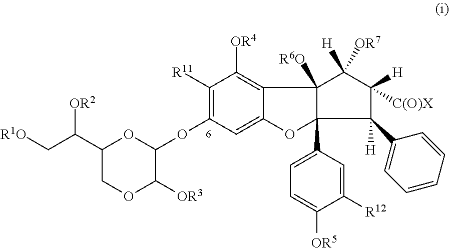

wherein
and each R1-R¹⁰ is independently selected from the group consisting of hydrogen, optionally substituted alkyl, optionally substituted acyl, optionally substituted aryl, optionally substituted arylalkyl, optionally substituted cycloalkylalkyl, optionally substituted arylacyl, optionally substituted cycloalkylacyl and a C-1 linked saccharide; and
X is OR⁸ or NR⁹ R¹⁰;
R¹¹ and R¹² are each independently hydrogen or, OR⁴ and R¹¹, and/or OR⁵ and R¹² together form a methylenedioxy group.

Examples of silvestrol analogs also include, but are not limited to, compounds (including stereoisomers within the dioxanyl group) of formula (i) or a salt or prodrug thereof.

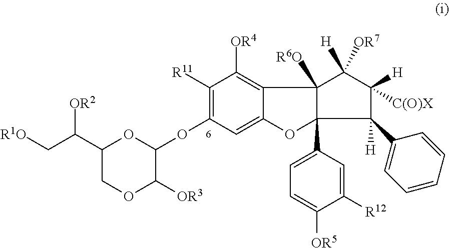

wherein each R1-R¹⁰ is independently selected from the group consisting of hydrogen, optionally substituted alkyl, optionally substituted acyl, optionally substituted aryl, optionally substituted arylalkyl, optionally substituted cycloalkylalkyl, optionally substituted arylacyl, optionally substituted cycloalkylacyl and a C-1 linked saccharide; and
X is OR⁸ or NR⁹ R¹⁰;
R¹¹ and R¹² are each independently hydrogen or, OR⁴ and R¹¹, and/or OR⁵ and R¹² together form a methylenedioxy group, wherein R¹¹ and R¹² are both hydrogen.

Additional examples of “silvestrol analogs” include, but are not limited to (−)-silvestrol, episilvestrol, (−)-episilvestrol, silvestrol dioxane, episilvesterol dioxane, Flavagline 61, (−)-4′-desmethoxyepisilvestrol and 1-O-formylaglafoline (or FA). Methods of making or synthesizing the silvestrol analogs can be found at least in Adams, T. E., et al, (2009). “Total synthesis of the potent anticancer Aglaia metabolites (−)-silvestrol and (−)-episilvestrol and the active analogue (−)-4′-desmethoxyepisilvestrol.” J. Am. Chem. Soc. 131, pp. 1607-1616; Bordeleau, M., et al. “Therapeutic suppression of translation initiation modulates chemosensitivity in a mouse lymphoma model.” J. Clin. Invest. Doi:10.1172/JC134753; Cencic, R., et al. (2009). “Antitumor activity and mechanism of action of the cyclopenta[b]benzofuran, silvestrol,” PLoS ONE 4(4): e5223. doi:10.1371/journal.pone.0005223; Cencic, R., et at. (2010). “Synergistic effect of inhibiting translation initiation in combination with cytotoxic agents in acute myelogenous leukemia cells.” Leukemia Research 34, pp. 535-541; El Sous, M., et al. (2007). “Total synthesis of (−)-episilvestrol and (−)-silvestrol.” Angew. Chem. Int. Ed. 46, pp. 1-5; U.S. Patent Application Publication No. 2008/0177093; U.S. Patent Application Publication No. 2009/0186839 and U.S. Pat. No. 6,710,075, all of which are hereby incorporated by reference for their leaching of silvestrol analogs and methods of making and synthesizing the same.

As used herein, “homolog” or “homologue” refers to a polypeptide or nucleic acid with homology to a specific known sequence. Specifically disclosed are variants of the nucleic acids and polypeptides herein disclosed which have at least 40, 41, 42, 43, 44, 45, 46, 47, 46, 49, 49, 50, 51, 52, 53, 54, 55, 56, 57, 56, 59, 60, 61, 62, 63, 64, 65, 66, 67, 68, 69, 70, 71, 72, 71, 74, 75, 76, 77, 78, 79, 80, 61, 82, 83, 84, 85, 86, 87, 68, 89, 90, 91, 92, 93, 94, 95, 96, 97, 98, 99 percent homology to the stated or known sequence. Those of skill in the art readily understand how to determine the homology of two proteins or nucleic acids. For example, the homology can be calculated after aligning the two sequences so that the homology is at its highest level. It is understood that one way to define any variants, modifications, or derivatives of the disclosed genes and proteins herein is through defining the variants, modification, and derivatives in terms of homology to specific known sequences.

As used herein, the term “subject” refers to the target of administration, e.g., an animal. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step. In some aspects of the disclosed method, the subject has been diagnosed with cancer prior to the administering step. In some aspects of the disclosed method, the subject has been diagnosed with a viral infection prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g. a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder” means having been subjected to a physical examination by a person of skill, for example, a physician, and found to have a condition that can be diagnosed or treated by a compound or composition that can treat the disorder. As a further example, “diagnosed with cancer” refers to having been subjected to a physical examination by a person of skill, for example, a physician, and found to have a condition characterized by cancer wherein treating the cancer would be beneficial to the subject. Such a diagnosis can be in reference to a disorder, such as cancer, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to cancer or a viral infection) based upon an earlier diagnosis by a person of skill, and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and i.e. of the patient; the time of administration; the route of administration; the rate of excretion of the specific compound employed; the duration of the treatment; drugs used in combination or coincidental with the specific compound employed and like factors well know in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for on or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “EC₅₀,” is intended to the concentration or dose of a substance (e.g., a compound or a drug) that is required for 50% enhancement or activation of a biological process, or component of a process, including a protein, subunit, organelle, ribonucleoprotein, etc. EC₅₀ also refers to the concentration or dose of a substance that is required for 50% enhancement or activation in vivo, as further defined elsewhere herein. Alternatively, EC₅₀ can refer to the concentration or dose of compound that provokes a response halfway between the baseline and maximum response. The response can be measured in an in vitro or in vivo system as is convenient and appropriate for the biological response of interest. For example, the response can be measured in vitro using cultured tumor cells or in an ex vivo organ culture system with isolated tumor cells. Alternatively, the response can be measured in vivo using an appropriate research model such as rodent, including mice and rats. The mouse or rat can be an inbred strain with phenotypic characteristics of interest such as obesity or diabetes. As appropriate, the response can be measured in a transgenic or knockout mouse or rat wherein the gene or genes has been introduced or knocked-out, as appropriate, to replicate a disease process.

As used herein, “IC₅₀,” is intended to refer to the concentration or dose of a substance (e.g., a compound or a drug) that is required for 50% inhibition or diminution of a biological process, or component of a process, including a protein, subunit, organelle, ribonucleoprotein, etc. IC₅₀ also refers to the concentration or dose of a substance that is required for 50% inhibition or diminution in viva, as further defined elsewhere herein. Alternatively, IC₅₀ also refers to the half maximal (50%) inhibitory concentration (IC) or inhibitory dose of a substance. The response can be measured in a in vitro or in vivo system as is convenient and appropriate for the biological response of interest. For example, the response can be measured in vitro using cultured tumor cells or in an ex vivo organ culture system with isolated tumor cells. Alternatively, the response can be measured in vivo using an appropriate research model such as rodent, including mice and rats. The mouse or rat can be an inbred strain with phenotypic characteristics of interest such as obesity or diabetes. As appropriate, the response can be measured in a transgenic or knockout mouse or rat wherein the a gene or genes has been introduced or knocked-out, as appropriate, to replicate a disease process.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As described, herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

As used herein, the term “stable” refers to compounds that are not substantially altered when subjected to conditions to allow for their production, detection, and, in certain aspects, their recovery, purification, and use for one or more of the purposes disclosed herein.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with en asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

It is known that chemical substances form solids which are present in different states of order which are termed polymorphic forms or modifications. The different modifications of a polymorphic substance, can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth heroin be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc., of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein tare certain structural requirements for performing the disclosed functions, and it is understood that there are a variety of structures that can perform the same function that are related to the disclosed structures, and that these structures will typically achieve the same result.

### A. Compositions

A composition comprising: (a) silvestrol or a silvestrol analog; (b) a anti-cancer agent; and (c) a pharmaceutically acceptable carrier.

A composition comprising: (a) silvestrol or a silvestrol analog; (b) anti-cancer agent; and (c) pharmaceutically acceptable carrier, wherein the silvestrol or silvestrol analog is silvestrol. In an aspect, disclosed herein is a composition comprising: (a) silvestrol or a silvestrol analog; (b) a anti-cancer agent; and (c) a pharmaceutically acceptable carrier, wherein the silvestrol or silvestrol analog is silvestrol wherein silvestrol has the following structure:

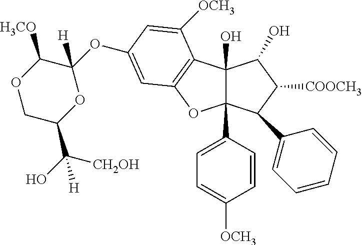 [SMILES: COC(=O)[C@H]1[C@@H](O)[C@@]2(O)c3c(OC)cc(O[C@@H]4O[C@@H]([C@H](O)CO)CO[C@H]4OC)cc3O[C@@]2(c2ccc(OC)cc2)[C@@H]1c1ccccc1]

A composition comprising: (a) silvestrol or a silvestrol analog; (b) a anti-cancer agent; and (c) to pharmaceutically acceptable carrier, wherein the silvestrol or silvestrol analog is silvestrol analog. In an aspect, disclosed herein is a composition comprising: (a) silvestrol or a silvestrol analog; (b) a anti-cancer agent; and (c) a pharmaceutically acceptable carrier, wherein the silvestrol analog is a compound of Formula (I) or a salt or prodrug thereof or a compound (including stereoisomers within the dioxanyl group) of formula (i) or a salt or prodrug thereof.

A composition comprising: (a) silvestrol or a silvestrol analog; (b) a anti-cancer agent; and (c) a pharmaceutically acceptable carrier, wherein the anti-cancer agent is an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

A composition comprising: (a) silvestrol or a silvestrol analog; (b) a anti-cancer agent; and (c) pharmaceutically acceptable carrier, wherein the composition further comprises one or more immunomodulatory agents.

A composition comprising; (a) silvestrol or a silvestrol analog; (b) a anti-cancer agent; and (c) to pharmaceutically acceptable carrier, wherein the one or more immunomodulatory agents is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, cytokine or receptor antagonist, or an adoptively transferred cell (NK, T cells, DCs).

A composition comprising: (a) silvestrol or a silvestrol analog; (b) a vaccine; and (c) a pharmaceutically acceptable carrier.

A composition comprising: (a) silvestrol or a silvestrol analog; (b) a vaccine; and (c) a pharmaceutically acceptable carrier, wherein the silvestrol or silvestrol analog is silvestrol. In an aspect, disclosed herein is a composition comprising: (a) silvestrol or a silvestrol analog; (b) a vaccine; and (c) a pharmaceutically acceptable carrier, wherein the silvestrol or silvestrol analog is silvestrol wherein silvestrol has the following structure:

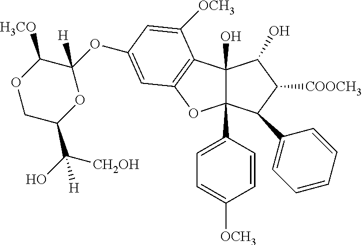 [SMILES: COC(=O)[C@H]1[C@@H](O)[C@@]2(O)c3c(OC)cc(O[C@@H]4O[C@@H]([C@H](O)CO)CO[C@H]4OC)cc3O[C@@]2(c2ccc(OC)cc2)[C@@H]1c1ccccc1]

A composition comprising: (a) silvestrol or a silvestrol analog; (b) a vaccine; and (c) a pharmaceutically acceptable carrier, wherein the silvestrol or silvestrol analog is a silvestrol analog. In an aspect, disclosed herein is a composition comprising: (a) silvestrol or a silvestrol analog; (b) a anti-cancer agent; and (c) a pharmaceutically acceptable carrier, wherein the silvestrol analog is a compound of Formula (I) or a salt or prodrug thereof or a compound (including stereoisomers within the dioxanyl group) of formula (i) or a salt or prodrug thereof.

A composition comprising; (a) silvestrol or a silvestrol analog; (b) a vaccine; and (c) a pharmaceutically acceptable carrier, wherein the vaccine is a tumor cell vaccines, antigen vaccines, dendritic cell vaccines, DNA vaccines, and vector-based vaccines.

A composition comprising: (a) silvestrol or a silvestrol analog; (b) a vaccine; and (c) a pharmaceutically acceptable carrier, wherein the composition further comprises one or more immunomodulatory agents.

A composition comprising: (a) silvestrol or a silvestrol analog; (b) a vaccine; and (c) a pharmaceutically acceptable carrier, wherein the one or more immunomodulatory agents is a cytokine, immune system adjuvant, fusion protein, an antibody, a preventive vaccine, a therapeutic vaccine, cytokine, a biologic fusion construct, a nucleic acid constructs (i.e.: DNA-based vaccines or immune modulatory products), a receptor ligand, cytokine or receptor antagonist, or an adoptively transferred cell (NK, T cells, DCs).

The vaccine in the compositions disclosed herein can be preventative (or prophylactic) vaccines and treatment or therapeutic vaccines. Examples of vaccines that can be used in the compositions and methods disclosed herein, include, but are not limited to, tumor cell vaccines, antigen vaccines, dendritic cell vaccines, DNA vaccines, and vector-based vaccines.

A composition comprising silvestrol or a silvestrol analog, and a pharmaceutically acceptable carrier, wherein the composition is provided in a dosage and frequency less than 0.3 mg/kg/day but effective to modulate the immune system in a subject. In an aspect, the composition is provided in a dosage and frequency 0.2 mg/kg/day. In an aspect, the composition is provided in a dosage and frequency 0.2 mg/kg every other day.

A composition comprising silvestrol or a silvestrol analog, and a pharmaceutically acceptable carrier, wherein the composition is provided in a dosage and frequency less than 0.3 mg/kg/day but effective to modulate the immune system in a subject, wherein the silvestrol or silvestrol analog is silvestrol. In an aspect, the composition is provided in a dosage and frequency 0.2 mg/kg/day. In an aspect, the composition is provided in a dosage and frequency 0.2 mg/kg every other day.

A composition comprising silvestrol or a silvestrol analog, and a pharmaceutically acceptable carrier, wherein the composition is provided in a dosage and frequency less than 0.3 mg/kg/day but effective to modulate the immune system in a subject, wherein the silvestrol or silvestrol analog is a silvestrol analog. In an aspect, the composition is provided in a dosage and frequency 0.2 mg/kg/day. In an aspect, the composition is provided in a dosage and frequency 0.2 mg/kg every other day.

A composition comprising silvestrol or a silvestrol analog, and a pharmaceutically acceptable carrier, wherein the composition is provided in a dosage and frequency less than 0.3 mg/kg/day but effective to modulate the immune system in a subject, wherein the silvestrol analog is a compound of Formula (I) or a salt or prodrug thereof or a compound (including stereoisomers within the dioxanyl group) of formula (i) or a salt or prodrug thereof. In an aspect, the composition is provided in a dosage and frequency 0.2 mg/kg/day. In an aspect, the composition is provided in a dosage and frequency 0.2 mg/kg every other day.

A composition comprising silvestrol or a silvestrol analog, and a pharmaceutically acceptable carrier, wherein the composition is provided in a dosage and frequency less than 0.3 mg/kg/day but effective to modulate the immune system in a subject, wherein the composition further comprises one or more immunomodulatory agents, wherein the one or more immunomodulatory agents is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, the composition is provided in a dosage and frequency 0.2 mg/kg/day. In an aspect, the composition is provided in a dosage and frequency 0.2 mg/kg every other day.

A combination therapeutic composition comprising silvestrol or a silvestrol analog, and an anti-viral agent.

A combination therapeutic composition comprising silvestrol or a silvestrol analog, and an anti-viral agent, wherein the silvestrol or silvestrol analog is silvestrol.

A combination therapeutic composition comprising silvestrol or silvestrol analog, and an anti-viral agent, wherein the silvestrol or silvestrol analog is a silvestrol analog.

A combination therapeutic composition comprising silvestrol or a silvestrol analog, and an anti-viral agent, wherein the silvestrol analog a compound of Formula (I) or a salt or prodrug thereof or a compound (including stereoisomers within the dioxanyl group) of formula (i) or a salt or prodrug thereof.

In a further aspect, the invention relates to compositions useful in methods to treat cancer, modulate the immune system, modulate viral gene expression, modulate the activity of an immune cell, or to treat a disease state or conditions associated with cellular hyperproliferation, and pharmaceutical compositions comprising compositions used in the methods.

In one aspect, the compositions of the invention are useful in the treatment of cancer. In one aspect, the compositions of the invention are useful in the prevention of cancer. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

A non-limiting list of different types of cancerous conditions is lung tumors, prostate tumors, colon tumors, brain tumors, melanoma tumors, ovarian tumors, renal tumors, breast tumors, leukemia, or hematologic malignancies.

A non-limiting list of different types of hematologic malignancy is CLL, ALL, MCL, AML, EBV-driven lymphoma, EBV+ lymphomas, nasopharngeal carcinoma, aggressive hematologic malignancies, Hodgkin's lymphomas, Burkitt's lymphoma, or diffuse large B cell lymphoma.

A non-limiting list of different types of disease state or conditions associated with cellular hyperproliferation is atherosclerosis, restinosis, rheumatoid arthritis, osteoarthritis, inflammatory arthritis, psoriasis, peridontal disease or virally induced cellular hyperproliferation.

In one aspect, the compositions of the invention are useful in the treatment of a viral infection. In one aspect, the compositions of the invention are useful in the prevention of a viral infection. In a further aspect, the viral infection can be a systemic, localized, acute, chronic, recurrent, or an asymptomatic viral infection. Symptoms of a viral infection include, but are not limited to fever, pain, headache, skin rash, or discharge. A cause of a viral infection can be often recognized from a combination of symptoms and signs. For example, a doctor can take a sample of blood, urine, stool, sputum, nasal or other discharge and send it to laboratory, where microbes can be determined by serologic tests or a culture.

In one aspect, the compositions of the invention are useful in modulating the immune system. In one aspect, the compositions of the invention are useful in modulating the activity of an immune cell.

In some aspects the anti-viral agent is a vaccine, nucleoside analogue (e.g. AZT, acyclovir, ganciclovir), an anti retroviral agents (e.g. nucleoside RT inhibitors, non nucleoside RT inhibitors, chemokine blockers (CCR5,), an integrase inhibitor, compositions in the HAART drug class, siRNA, shRNA, anti-herpetic agents, anti-influenza agents, anti-encephalitis agents, anti-hepatitis agents, anti-labyrynthitis agents, anti-lymphoid interstitial pneumonia agents, anti-meningitis agents, anti-orf agents, anti-pneumonia agents, anti-Ramsay Hunt Syndrome Type II agents, anti-SARS agents, anti-shingles agents, anti-Epstein Barr virus agents, anti-EBV agents, anti-HSV agents, anti-HPV agents, adamantane anti-virals, anti-viral combinations, anti-viral interferons, chemokine receptor antagonist, integrase strand transfer inhibitor, miscellaneous anti-virals, neuraminidase inhibitors, NNRTIs, nucleoside reverse transcriptase inhibitors (NRTIs), protease inhibitors, or purine nucleosides. Additional anti-viral agents include, but are not limited to, anti-HIV, NRTI drug, “Ziagen” (ViiV Healthcare), “Trizivir”, “Kivexa/Epzicom”, Aciclovir, anti-HSV, Acyclovir, Adefovir, Amantadine, Amprenavir, Ampligen, Arbidol, Atazanavir, Atripla, Boceprevir, Cidofovir, Combivir, Darunavir, Delavirdine, Didanosine, Docosanol, Edoxudine, Efavirenz, Emtricitabine, Enfuvirtide, Entecavir, Entry inhibitors, Famciclovir, Fusion inhibitor, Ganciclovir, Ibacitabine, Imunovir, Idoxuridine, Imiquimod, Indinavir, Inosine, Integrase inhibitors, Interferon type III, Interferon type II, Interferon type I, Interferon Lamivudine, Lopinavir, Loviride, Maraviroc, Moroxydine, Methisazone, Nelfinavir, Nevirapine, Nexavir, Nucleoside analogues, Oseltamivir (Tamiflu), Peginterferon alfa-2a, Penciclovir, Peramivir, Pleconaril, Podophyllotoxin, Protease inhibitors, Raltegravir, Reverse transcriptase inhibitors, Ribavirin, Rimantadine, Ritonavir, Pyramidine, Saquinavir, Stavudine, Synergistic enhancers, Tea tree oil, Telaprevir, Tenofovir, Tenofovir disoprosil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compositions can be employed in the disclosed methods of using.

i) Silvestrol or Silvestrol Analog and an Anti-Cancer Agent

Disclosed herein are compositions for treating or preventing cancer, the composition comprising: (a) silvestrol or a silvestrol analog; (b) a anti-cancer agent; and (c) a pharmaceutically acceptable carrier, wherein the composition is administered in a dosage and frequency synergistically effective to treat the disorder in the subject. In an aspect, the composition comprises a therapeutically effective amount of silvestrol or a silvestrol analog and an anti-cancer agent. In an aspect, the composition comprises a prophylactically effective amount of silvestrol or a silvestrol analog and an anti-cancer agent. In an aspect, the amount of silvestrol or a silvestrol analog in the composition is less than 0.3 mg/kg. In a further aspect, the amount of silvestrol or a silvestrol analog in the composition less than 0.2 mg/kg. In an aspect, the amount of silvestrol or a silvestrol analog in the composition is 0.2 mg/kg. In an even further aspect, the amount of silvestrol or a silvestrol analog in the composition is 0.1 mg/kg.

Disclosed herein are compositions for vaccinating a subject not yet diagnosed with cancer, the composition comprising: (a) silvestrol or a silvestrol analog; (b) a anti-cancer agent; and (c) a pharmaceutically acceptable carrier, wherein the composition is administered in a dosage and frequency synergistically effective to treat the disorder in the subject. In an aspect, the composition comprises a therapeutically effective amount of siivestroi or a silvestrol analog and an anti-cancer agent. In an aspect, the composition comprises a prophylactically effective amount of silvestrol or a silvestrol analog and an anti-cancer agent. In an aspect, the amount of silvestrol or a silvestrol analog in the composition is less than 0.3 mg/kg. In a further aspect, the amount of silvestrol or a silvestrol analog in the composition is less than 0.2 mg/kg. In an aspect, the amount of silvestrol or a silvestrol analog in the composition is 0.2 mg/kg. In an even further aspect, the amount of silvestrol or a silvestrol analog in the composition is 0.1 mg/kg.

ii) Silvestrol or Silvestrol Analog and a Vaccine

Disclosed herein are compositions for treating or preventing cancer, the composition comprising: (a) silvestrol or a silvestrol analog; (b) a vaccine; and (c) pharmaceutically acceptable carrier, wherein the composition is administered in a dosage and frequency synergistically effective to treat the disorder in the subject. In an aspect, the composition comprises a therapeutically effective amount silvestrol or a silvestrol analog, and a vaccine. In an aspect, the composition comprises a prophylactically effective amount of silvestrol or a silvestrol analog and a vaccine. In an aspect, the amount of silvestrol or a silvestrol analog in the composition is less than 0.3 mg/kg. In a further aspect, the amount of silvestrol a silvestrol analog in the composition less than 0.2 mg/kg. In an aspect, the amount of silvestrol or a silvestrol analog in the composition is 0.2 mg/kg. In an even further aspect, the amount of silvestrol or a silvestrol analog in the composition is 0.1 mg/kg. In an aspect, the vaccine is a tumor cell vaccine, antigen vaccine, dendritic cell vaccine, DNA vaccine, or a vector-based vaccine.

Disclosed herein are compositions for vaccinating a subject not yet diagnosed with cancer, the composition comprising: (a) silvestrol or a silvestrol analog; (b) a vaccine; and (c) a pharmaceutically acceptable carrier, wherein the composition is administered in a dosage and frequency synergistically effective to treat the disorder in the subject. In an aspect, the composition comprises a therapeutically effective amount of silvestrol or a silvestrol analog and a vaccine. In an aspect, the composition comprises a prophylactically effective amount of silvestrol or a silvestrol analog and a vaccine. In an aspect, the amount of silvestrol or a silvestrol analog in the composition is less than 0.3 mg/kg. In a further aspect, the amount of silvestrol or a silvestrol analog in the composition less than 0.2 mg/kg. In an aspect, the amount of silvestrol or a silvestrol analog in the composition is 0.2. mg/kg. In an even further aspect, the amount of silvestrol or a silvestrol analog in the composition is 0.1 mg/kg. In an aspect, the vaccine is a tumor cell vaccine, antigen vaccine, dendritic cell vaccine, DNA vaccine, or a vector-based vaccine.

iii) Silvestrol or Silvestrol Analog and an Anti-Viral Agent

Disclosed herein are compositions for treating, or preventing a viral infection, the composition comprising: (a) silvestrol or a silvestrol analog; (b) anti-viral agent; and (c) a pharmaceutically acceptable carrier, wherein the composition is administered in a dosage and frequency synergistically effective to treat the disorder in the subject. In an aspect, the composition comprises a therapeutically effective amount of silvestrol or a silvestrol analog and an anti-viral agent. In an aspect, the composition comprises a prophylactically effective amount of silvestrol or a silvestrol analog and anti-viral agent. In an aspect, the amount of silvestrol or a silvestrol analog in the composition is less than 0.3 mg/kg. In a further aspect, the amount of silvestrol or a silvestrol analog in the composition less than 0.2 mg/kg. In an aspect, the amount of silvestrol or a silvestrol analog in the composition is 0.2 mg/kg. In an even further aspect, the amount of silvestrol or a silvestrol analog in the composition is 0.1 mg/kg.

Disclosed herein are compositions for vaccinating a subject against a viral infection, the composition comprising: (a) silvestrol or a silvestrol analog; (b) an anti-viral agent; and (c) a pharmaceutically acceptable carrier, wherein the composition is administered in a dosage and frequency synergistically effective to treat the cancer in the subject. In an aspect, the composition comprises a therapeutically effective amount of silvestrol or a silvestrol analog and an anti-viral agent. In an aspect, the composition comprises a prophylactically effective amount of silvestrol or a silvestrol analog and an anti-viral agent. In an aspect, the amount of silvestrol or a silvestrol analog in the composition is less than 0.3 mg/kg. In a further aspect, the amount of silvestrol or a silvestrol analog in the composition less than 0.2 mg/kg. In an aspect, the amount of silvestrol or a silvestrol analog in the composition is 0.2 mg/kg. In an even further aspect, the amount of silvestrol or a silvestrol analog in the composition is 0.1 mg/kg. In an aspect, the anti-viral agent is a vaccine, wherein the vaccine is a tumor cell vaccine, antigen vaccine, dendritic cell vaccine, DNA vaccine, or a vector-based vaccine.

In some aspects the anti-viral agent is a vaccine, nucleoside analoge (e.g. AZT, acyclovir, ganciclovir), an anti retroviral agents (e.g. nucleoside RT inhibitors, non nucleoside RT inhibitors, chemokine blockers (CCR5,), an integrase inhibitor, compositions in the HAART drug class, siRNA shRNA anti-herpetic agents, anti-influenza agents, anti-encephalitis agents, anti-hepatitis agents, anti-labyrynthitis agents, anti-lymphoid interstitial pneumonia agents, anti-meningitis agents, anti-orf agents, anti-pneumonia agents, anti-Ramsay Hunt Syndrome Type II agents, anti-SARS agents, anti-shingles agents, anti-Epstein Barr virus agents, anti-EBV agents, anti-HSV agents, anti-HPV agents, adamantane anti-virals, anti-viral combinations, anti-viral interferons, chemokine receptor antagonist, integrase strand transfer inhibitor, miscellaneous anti-virals, neuraminidase inhibitors, NNRTIs, nucleoside reverse transcriptase inhibitors (NRTIs), protease inhibitors, or purine nucleosides.

It is contemplated that one or more compositions can optionally be omitted from the disclosed invention.

### B. Methods of Making the Compound

In one aspect, the disclosed compounds comprise the products of the synthetic methods described herein. In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

The compounds of the invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein. The following examples are provided so that the invention might be more fully understood, are illustrative only, and should not be construed as limiting.

iv) Synthesis

It is further anticipated that the compounds of the invention can be obtained by direct synthesis. Direct synthesis may include either total synthesis or semi-synthesis. Exemplary synthetic methods for obtaining these compounds are described above.

It is contemplated that each disclosed methods can further comprise additional steps, manipulations, and/or components. It is also contemplated that any one or more step, manipulation, and/or component can be optionally omitted from the invention. It is understood that a disclosed methods can be used to provide the disclosed compounds. It is also understood that the products of the disclosed methods can be employed in the disclosed methods of using.

C. Pharmaceutical Compositions

In one aspect, the invention relates to pharmaceutical compositions comprising the disclosed compositions comprising (a) silvestrol or a silvestrol analog; (b) an anti-cancer agent; and (c) a pharmaceutically acceptable carrier. In a further aspect, the invention relates to pharmaceutical compositions comprising the disclosed compositions comprising (a) silvestrol a silvestrol analog; (b) a vaccine; and (c) a pharmaceutically acceptable carrier. In a further aspect, the invention relates to pharmaceutical compositions comprising the disclosed composition comprising a combination therapeutic composition comprising silvestrol or a silvestrol analog, and an anti-viral agent. In an aspect, the disclosed pharmaceutical compositions can be provided comprising a therapeutically effective amount. The disclosed pharmaceutical compositions can be provided comprising a prophylactically effective amount.

In a further aspect, the pharmaceutical composition is administered following identification of the mammal in need of treatment of cancer. In a still further aspect, the pharmaceutical composition is administered following identification of the mammal in need of prevention of cancer. In an even further aspect, the mammal has been diagnosed with a need for treatment of cancer prior to the administering step.

In a further aspect, the pharmaceutical composition is administered following identification of the mammal in need of treatment of a viral infection. In a still further aspect, the pharmaceutical composition is administered following identification of the mammal in need of prevention of a viral infection. In an even further aspect, the mammal has been diagnosed with a need for treatment of a viral infection prior to the administering step.

In certain aspects, the disclosed pharmaceutical compositions comprise the disclosed (including, pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salt derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, di.e.thylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, ethanesulfonic, fumaric, gluconic, glutamic, hydrobromic, hydrochloric, isethionic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueons liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Farther, the compositions can be in a form suitable for use in transdermal devices. These formulations can be prepared, utilizing a compound of the invention, or pharmaceutically acceptable salts thereof, via conventional processing methods, an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require modulation of the immune system an appropriate dosage level will generally be about 0.1 l to 0.5 mg per kg patient body weight per day and can be administered in single or multiple doses. Preferably, the dosage level will be about 0.1 to about 0.4 mg/kg per day; more preferably 0.2 to 0.3 mg/kg per day. The compound can be administered on a regimen of 1 to 4 times per day. In some aspects, the administration can be daily, every other day, every third day, every forth day, every fifth day, every sixth day, or once per week. This dosing regimen can be adjusted to provide the optimal therapeutic response.

It is understood, however, that the specific dose level for any particular patient will depend upon a variety of factors. Such factors include the age, body weight, general health, sex, and i.e. of the patient. Other factors include the time and route of administration, rate of excretion, drug combination, and the type and severity of the particular disease undergoing therapy.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

### D. Methods of Using the Compositions

i) Treatment Methods with Anticancer Agents

Disclosed herein are treatment methods comprising the step of administering, to a subject diagnosed with a cancer, a composition comprising; (a) silvestrol or a silvestrol analog; (b) an anti-cancer agent; and (c) a pharmaceutically acceptable carrier, wherein the composition is administered in a dosage and frequency synergistically effective to treat the cancer in the subject. In an aspect, disclosed herein are treatment methods comprising the step of administering, to a subject diagnosed with a cancer, a composition comprising; (a) silvestrol or a silvestrol analog; (b) an anti-cancer agent, and (c) a pharmaceutically acceptable carrier, wherein the composition is administered in a dosage and frequency synergistically effective to treat the cancer in the subject, wherein the anti-cancer agent is an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells , DCs).

In an aspect, disclosed herein are treatment methods comprising the step of administering, to a subject diagnosed with a cancer, a composition comprising: (a) silvestrol or a silvestrol analog; (b) an anti-cancer agent, and (c) a pharmaceutically acceptable carrier, wherein the composition is administered in a dosage and frequency synergistically effective to treat the cancer in the subject, wherein the dosage of the anti-cancer agent is lower than the standard amount necessary to treat the cancer in the subject. In some aspects, the dosage of the anti-cancer agent is 5%, 10%, 15%, or 20% lower than the standard amount of the anti-cancer agent when administered without silvestrol or a silvestrol analog necessary to treat the cancer in the subject.

In an aspect, disclosed herein are treatment methods comprising the step of administering, to a subject diagnosed with a cancer, a composition comprising: (a) silvestrol or a silvestrol analog; (b) an anti-cancer agent, and (c) a pharmaceutically acceptable carrier, wherein the composition is administered in a dosage and frequency synergistically effective to treat the cancer in the subject, wherein the silvestrol analog is a compound of Formula (I) or a salt or prodrug thereof or a compound (including stereoisomers within the dioxanyl group) of formula (i) or a salt or prodrug thereof.

In an aspect, disclosed herein are treatment methods comprising the step of administering, to a subject diagnosed with a cancer, a composition comprising: (a) silvestrol or a silvestrol analog, (b) anti-cancer agent; and (c) a pharmaceutically acceptable carrier, wherein the composition is administered in a dosage and frequency synergistically effective to treat the cancer in the subject, wherein the composition is administered in an amount in the range of 0.1 to 200 micrograms of complex per kg body weight of the subject per administration. In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the silvestrol or a silvestrol analog of the composition is administered in an amount in the range of 0.1 to 0.3 micrograms of complex per kg body weight of the subject per administration. In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the silvestrol or a silvestrol analog of the composition is administered in an amount of 0.2 micrograms of complex per kg body weight of the subject per administration.

In an aspect, disclosed herein are treatment methods comprising the step of administering, to a subject diagnosed with a cancer, a composition comprising: (a) silvestrol or a silvestrol analog, (b) an anti-cancer agent; and (c) a pharmaceutically acceptable carrier, wherein the composition is administered in a dosage and frequency synergistically effective to treat the cancer in the subject, wherein the composition is administered repeatedly to the subject. In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the subject is a mammal such as a human. In some aspects, the repeated administration can be daily, every other day, every third day, every forth day, every filth day, every sixth day, or once per week. One of skill will be able to determine dosage regime based on the dosage of the compositions given to the subject and the subject him or herself.

In an aspect, disclosed herein are treatment methods comprising the step of administering, to a subject diagnosed with a cancer, a composition comprising: (a) silvestrol or a silvestrol analog; (b) anti-cancer agent; and (c) a pharmaceutically acceptable carrier, wherein the composition is administered in a dosage and frequency synergistically effective to treat the cancer in the subject, wherein the subject has been diagnosed with a need for modulating the immune system prior to the administering step.

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, further comprising the step of identifying a subject in need of modulating the immune system.

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the cancer is lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the cancer is lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer. In some aspects, the cancer can be a cancerous condition including, but not limited to the presence of lung tumors, prostate tumors, colon tumors, brain tumors, melanoma tumors, ovarian tumors, renal tumors, breast tumors, leukemia, or hematologic malignancies. Hematologic malignancies include, but are not limited to CLL, ALL, MCL, AML, EBV-driven lymphoma, EBV+ lymphomas, nasopharngeal carcinoma, aggressive hematologic malignancies. Hodgkin's lymphomas, Burkitt's lymphoma, or diffuse large B cell lymphoma.

In an aspect, disclosed herein are treatment methods comprising the step of administering, to a subject diagnosed with a cancer, a composition comprising: (a) silvestrol or a silvestrol analog; (b) an anti-cancer agent; and (c) as pharmaceutically acceptable carrier, wherein the composition is administered in a dosage and frequency synergistically effective to treat the cancer in the subject, wherein humoral immune function in the subject is unaffected, wherein Ig production in the subject is unaffected, wherein IFNg production in the subject remains normal, wherein the composition potentiates virus-specific or tumor-specific cytotoxic T lymphocytes, wherein the composition potentiates virus-specific or tumor-specific T-helper cells, wherein the composition potentiates virus-specific or tumor-specific T-helper cells, or wherein the composition potentiates virus-specific or tumor-specific Th1, Th2, Th17 or Treg cells.

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the composition further comprises one or more immunomodulatory agents.

In an aspect, disclosed herein are treatment methods comprising the step of administering, to a subject diagnosed with a cancer, a composition comprising; (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein the one or more immunomodulatory agents is a cytokine, immune system adjuvant, fusion protein, an antibody, a preventive vaccine, a therapeutic vaccine a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

In an aspect, disclosed herein are treatment methods comprising the step of administering, to a subject diagnosed with a cancer, a composition comprising: (a) silvestrol or a silvestrol analog; (b) an anti-cancer agent; and (c) a pharmaceutically acceptable carrier, wherein the composition is administered in a dosage and frequency synergistically effective to treat the cancer in the subject, wherein one or more immunomodulatory agents potentiates an antibody-dependent cell-mediated cytoxicity (“ADCC”) response in the subject.

ii) Treatment Methods with a Vaccine

Disclosed herein are treatment methods comprising the step of administering, to a subject diagnosed with a cancer, a composition comprising; (a) silvestrol or a silvestrol analog; (b) a vaccine; and (c) a pharmaceutically acceptable carrier, wherein the composition is administered in a dosage and frequency synergistically effective to treat the cancer in the subject. In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to treat the cancer in the subject, wherein the vaccine is a tumor cell vaccines, antigen vaccines, dendritic cell vaccines, DNA vaccines, and vector-based vaccines.

In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to treat the cancer in the subject, wherein time dosage of the vaccine is lower than the standard amount necessary to treat the cancer in the subject. In some aspects, the dosage of the vaccine is 5%, 10%, 15%, or 20% lower than the standard amount of the vaccine when administered without slivestrol or a silvestrol analog necessary to treat the cancer in the subject.

In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to treat the cancer in the subject, wherein the silvestrol analog is a compound of Formula (I) or a salt or prodrug thereof or a compound (including stereoisomers within the dioxanyl group) of formula (i) or a salt or prodrug thereof.

In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to treat the cancer in the subject, wherein the composition is administered in an amount in the range of 0.1 to 200 micrograms of complex per kg body weight of the subject per administration. In an aspect, disclosed herein are treatment methods comprising the step of administering, to a subject diagnosed with a cancer, a composition comprising: (a) silvestrol or a silvestrol analog; (b) a vaccine; and (v) a pharmaceutically acceptable carrier, wherein the composition is administered in a dosage and frequency synergistically effective to treat the cancer in the subject, wherein the silvestrol or a silvestrol analog of the composition is administered in an amount in the range of 0.1 to 0.3 micrograms of complex per kg body weight of the subject per administration. In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered is a dosage and frequency synergistically effective to treat the cancer in the subject, wherein the silvestrol or a silvestrol analog of the composition is administered in an amount of 0.2 micrograms of complex per kg body weight of the subject per administration.

In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to treat the cancer in the subject, wherein the composition is administered repeatedly to the subject. In an aspect, disclosed herein are treatment methods comprising the step of administering, to a subject diagnosed with a cancer, a composition comprising: (a) silvestrol or a silvestrol analog; (b) a vaccine and (c) a pharmaceutically acceptable carrier, wherein the composition is administered in a dosage and frequency synergistically effective to treat the cancer in the subject, wherein the subject is a mammal such as a human. In some aspects, the repeated administration can be daily, every other day, every third day, every forth day, every fifth day, every sixth day, or once per week. One of skill will be able to determine dosage regime based on the dosage of the compositions given to the subject and the subject him or herself.

In an aspect, disclosed herein are treatment methods comprising the step of administering, to a subject diagnosed with a cancer, a composition comprising: (a) silvestrol or a silvestrol analog, (b) a vaccine; and (c) a pharmaceutically acceptable carrier, wherein the composition is administered in a dosage and frequency synergistically effective to treat the cancer in the subject, wherein the subject has been diagnosed with, a need for modulating the immune system prior to the administering step,

In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to treat the cancer in the subject, further comprising the step of identifying a subject in need of modulating the immune system.

In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to treat the cancer in the subject, wherein the cancer is lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers, testicular cancer; colon and rectal cancers, prostatic cancer, and pancreatic cancer.

In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to treat the cancer in the subject, wherein humoral immune function in the subject is unaffected, wherein Ig production in the subject is unaffected, wherein IFNg production in the subject remains normal, wherein the composition potentiates virus-specific or tumor-specific cytotoxic T lymphocytes, wherein the composition potentiates virus-specific or tumor-specific T-helper cells, wherein the composition potentiates virus-specific or tumor-specific T-helper cells, or wherein the composition potentiates virus--specific or tumor-specific Th1, Th2, Th17, or Treg cells.

In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to treat the cancer in the subject, wherein the composition further comprises one or more immunomodulatory agents.

In an aspect, disclosed herein are treatment methods comprising the step of administering, to a subject diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to treat the cancer in the subject, wherein the one or more immunomodulatory agents is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

In an aspect, disclosed herein are treatment methods comprising the step of administering, to a subject diagnosed with a cancer, a composition comprising: (a) silvestrol or a silvestrol analog; (b) a vaccine; and (c) i pharmaceutically acceptable carrier, wherein the composition is administered in a dosage and frequency synergistically effective to treat the cancer in the subject, wherein one or more immunomodulatory agents potentiates an antibody-dependent cell-mediated cytoxicity (“ADCC”) response in the subject.

iii) Vaccination Methods

Disclosed herein are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer. In an aspect, disclosed herein are vaccination methods comprising the step of administering, to a subject not yet diagnosed with a cancer, a composition comprising: (a) silvestrol or a silvestrol analog; (b) anti-cancer agent; and (c) a pharmaceutically acceptable carrier, wherein the composition is administered in a dosage and frequency synergistically effective to modulate the immune system in the subject, thereby vaccinating the subject against the cancer wherein the anti-cancer agent is an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer, wherein the dosage of the anti-cancer agent is lower than the standard amount necessary to treat the cancer in the subject. In some aspects, the dosage of the anti-cancer agent is 5%, 10%, 15%, or 20% lower than the standard amount of the anti-cancer agent when administered without silvestrol or a silvestrol analog necessary to treat the cancer in the subject.

In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer wherein the silvestrol analog is a compound of Formula (I) or a salt or prodrug thereof or a compound (including stereoisomers within the dioxanyl group) of formula (i) or a salt or prodrug thereof.

In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer wherein the composition is administered in an amount in the range of 0.1 to 200 micrograms of complex per kg body weight of the subject per administration. In an aspect, are vaccination methods comprising the step of administering, to a subject not yet diagnosed with a cancer, a composition comprising: (a) silvestrol or silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer wherein the silvestrol or silvestrol analog of the composition is administered in an amount in the range of 0.1 to 0.3 micrograms of complex per kg body weight of the subject per administration.

In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer wherein the composition is administered repeatedly to the subject. In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer wherein the subject is a mammal, such as a human.

In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer, wherein the subject has been diagnosed with a need for modulating the immune system prior to the administering step.

In an aspect, are vaccination methods comprising the step of administering, to a subject not yet diagnosed with a cancer, a composition comprising: (a) silvestrol or a silvestrol analog; (b) a anti-cancer agent; and (c) pharmaceutically acceptable carrier, wherein the composition is administered in a dosage and frequency synergistically effective to modulate the immune system in the subject, thereby vaccinating the subject against the cancer, further comprising the step of identifying a subject in need of modulating the immune system. In an aspect, are vaccination methods comprising the step of administering, to a subject not yet diagnosed with a cancer, a composition comprising; (a) silvestrol or a silvestrol analog; (b) anti-cancer agent; and (c) pharmaceutically acceptable carrier, wherein the composition is administered in a dosage and frequency synergistically effective to modulate the immune system in the subject, thereby vaccinating the subject against the cancer, wherein humoral immune function in the subject is unaffected, wherein Ig production in the subject is unaffected, wherein IFNg production in the subject remains normal, wherein the composition potentiates virus-specific or tumor-specific cytotoxic T lymphocytes, wherein the composition potentiates virus-specific or tumor-specific T-helper cells, wherein the composition potentiates virus-specific or tumor-specific T-helper cells, or wherein the composition potentiates virus-specific or tumor-specific Th1, Th2, Th17, or Treg cells.

In an aspect, are vaccination methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject, thereby vaccinating the subject against the cancer, wherein the composition further comprises one or more immunomodulatory agents. In an aspect, the one or more immunomodulatory agents is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, are methods comprising the step of administering, to a subject not yet diagnosed with a cancer, a composition comprising: (a) silvestrol or a silvestrol analog; (b) a anti-cancer agent; and (c) a pharmaceutically acceptable carrier, wherein the composition is administered in a dosage and frequency synergistically effective to modulate the immune system in the subject thereby vaccinating the subject against the cancer, wherein the composition further comprises one or more immunomodulatory agents, wherein one or more immunomodulatory agents potentiates an antibody-dependent cell-mediated cytoxicity (“ADCC”) response in the subject.

Disclosed herein are vaccination methods comprising the step of administering, to a subject not yet diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer. In an aspect, disclosed herein are vaccination methods comprising the step of administering, to a subject not yet diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer wherein the anti-cancer agent is an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs).

In an aspect, are vaccination methods comprising the step of administering, to a subject not yet diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer, wherein the dosage of the anti-cancer agent is lower than the standard amount necessary to treat the cancer in the subject. In some aspects, the dosage of the vaccine is 5%, 10%, 15%, or 20% lower than the standard amount of the vaccine when administered without silvestrol or a silvestrol analog necessary to treat the cancer in the subject.

In an aspect, are vaccination methods comprising the step of administering, to a subject not yet diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer, wherein the silvestrol analog is a compound of Formula (I) or a salt or prodrug thereof or a compound (including stereoisomers within the dioxanyl group) of formula (i) or a salt or prodrug thereof.

In an aspect, are vaccination methods comprising the step of administering, to a subject not yet diagnosed with a cancer, a composition comprising; (a) silvestrol or a silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer wherein the composition is administered in an amount in the range of 0.1 to 200 micrograms of complex per kg body weight of the subject per administration. In an aspect, are vaccination methods comprising the step of administering, to a subject not yet diagnosed with a cancer, a composition comprising: (a) silvestrol or silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer wherein the silvestrol or silvestrol analog of the composition is administered in an amount in the range of 0.1 to 0.3 micrograms of complex per kg body weight of the subject per administration.

In an aspect, are vaccination methods comprising the step of administering, to a subject not yet diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer wherein the composition is administered repeatedly to the subject. In an aspect, are vaccination methods comprising the step of administering, to a subject not yet diagnosed with a cancer, a composition comprising; (a) silvestrol or a silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer wherein the subject is a mammal, such as a human.

In an aspect, are vaccination methods comprising the step of administering, to a subject not yet diagnosed with a cancer, a composition comprising; (a) silvestrol or a silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer, wherein the subject has been diagnosed with a need for modulating the immune system prior to the administering step.

In an aspect, are vaccination methods comprising the step of administering, to a subject not yet diagnosed with a cancer, a composition comprising; (a) silvestrol or a silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer, further comprising the step of identifying a subject in need of modulating the immune system. In an aspect, are vaccination methods comprising the step of administering, to a subject not yet diagnosed with a cancer, a composition comprising: (a) silvestrol or a silvestrol analog; (b) a vaccine; and (c) pharmaceutically acceptable carrier, wherein the composition is administered in a dosage and frequency synergistically effective to modulate the immune system in the subject, thereby vaccinating the subject against the cancer, wherein humoral immune function in the subject is unaffected, wherein Ig production in the subject is unaffected, wherein IFNg production in the subject remains normal, wherein the composition potentiates virus-specific or tumor-specific cytotoxic T lymphocytes, wherein the composition potentiates virus-specific or tumor-specific T-helper cells, wherein the composition potentiates virus-specific or tumor-specific T-helper cells, of wherein the composition potentiates virus-specific or tumor-specific Th1, Th2, Th17, or Treg cells.

In an aspect, are vaccination methods comprising the step of administering, to a subject not yet diagnosed with a cancer, a composition comprising: (a) silvestrol or a silvestrol analog; (b) a vaccine; and (c) a pharmaceutically acceptable carrier, wherein the composition is administered in a dosage and frequency synergistically effective to modulate the immune system in the subject, thereby vaccinating the subject against the cancer, wherein the composition further comprises one or more immunomodulatory agents. In an aspect, the one or more immunomodulatory agents is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, are methods comprising the step of administering, to a subject not yet diagnosed with a cancer, a composition comprising: (a) silvestrol or a silvestrol analog (b) a vaccine; and (c) pharmaceutically acceptable carrier, wherein the composition is administered in a dosage and frequency synergistically effective to modulate the immune system in the subject, thereby vaccinating the subject against the cancer, wherein the composition further comprises one or more immunomodulatory agents, wherein the one or more immunomodulatory agents potentiates an antibody-dependent cell-mediated cytoxicity (“ADCC”) response in the subject.

iv) Methods of Administering Specific Dosages

Disclosed herein are methods comprising the step of administering a composition comprising silvestrol or silvestrol analog, and a pharmaceutically acceptable carrier, wherein the composition is administered in a dosage and frequency less than 0.5, 0.4, or 0.3 mg/kg/day. In an aspect, disclosed herein are methods comprising the step of administering a composition comprising silvestrol or a silvestrol analog and a pharmaceutically acceptable carrier, wherein the silvestrol or silvestrol analog of the composition is administered in a dosage and frequency less than 0.5, 0.4, or 0.3 mg/kg/day. In some aspects the dosage is 0.2 mg/kg/day.

In an aspect, disclosed herein are methods comprising the step of administering a composition comprising silvestrol or a silvestrol analog, and a pharmaceutically acceptable carrier, wherein the composition is administered in a dosage and frequency less than 0.5, 0.4, or 0.3 mg/kg/day, wherein the subject has been diagnosed with a cancer prior to administration. In an aspect, disclosed herein are methods comprising the step of administering a composition comprising silvestrol or a silvestrol analog, and a pharmaceutically acceptable carrier, wherein the silvestrol or silvestrol analog composition is administered in a dosage and frequency less than 0.5, 0.4, or 0.3 mg/kg/day, wherein the subject has been diagnosed with a cancer prior to administration. In some aspects the dosage is 0.2 mg/kg/day.

In an aspect, disclosed herein are methods comprising the step of administering a composition comprising silvestrol or a silvestrol analog, and a pharmaceutically acceptable carrier, wherein the composition is administered in a dosage and frequency less than 0.5, 0.4, or 0,3 mg/kg/day, wherein the silvestrol analog is a compound of Formula (I) or a salt or prodrug thereof or a compound (including stereoisomers within the dioxanyl group) of formula (i) or a salt or prodrug thereof. In some aspects the dosage is 0.2 mg/kg/day.

In an aspect, disclosed herein are methods comprising the step of administering a composition comprising silvestrol or a silvestrol analog, and a pharmaceutically acceptable carrier, wherein the silvestrol analog of the composition is administered in a dosage and frequency less than 0.5, 0.4, or 0.3 mg/kg/day, wherein the silvestrol analog is a compound of Formula (I) or a salt or prodrug thereof or a compound (including stereoisomers within the dioxanyl group) of formula (i) or a salt or prodrug thereof. In some aspects the dosage is 0.2 mg/kg/day.

In an aspect, disclosed herein are methods comprising the step of administering a composition comprising silvestrol or a silvestrol analog, and a pharmaceutically acceptable carrier, wherein the composition is administered in a dosage and frequency less than 0.5, 0.4, or 0.3 mg/kg/day, wherein the composition further comprises one or more immunomodulatory agents. In some aspects the dosage is 0.2 mg/kg/day.

In an aspect, disclosed herein are methods comprising the step of administering a composition comprising silvestrol or a silvestrol analog, and a pharmaceutically acceptable carrier, wherein the silvestrol or silvestrol analog of the composition is administered in a dosage and frequency less than 0.5, 0.4, or 0.3 mg/kg/day, wherein the composition further comprises one or more immunomodulatory agents. In some aspects the dosage is 0.2 mg/kg/day.

In an aspect, disclosed herein are methods comprising the step of administering a composition comprising silvestrol or a silvestrol analog, and a pharmaceutically acceptable carrier, wherein the composition is administered in a dosage and frequency less than 0.5, 0.4, or 0.3 mg/kg/day, wherein the composition further comprises one or more immunomodulatory agents, wherein the one or more immunomodulatory agents is a cytokine, immune system adjuvant, fusion protein, an antibody, a preventive vaccine, a therapeutic vaccine, a cytokine, biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, disclosed herein are methods comprising the step of administering a composition comprising silvestrol or a silvestrol analog, and a pharmaceutically acceptable carrier, wherein the silvestrol or silvestrol analog, composition is administered in a dosage and frequency less than 0.5, 0.4, or 0.3 mg/kg/day, wherein the composition further comprises one or more immunomodulatory agents, wherein the one or more immunomodulatory agents is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, cytokine or receptor antagonist, or an adoptively transferred cell (NK, T cells, DCs). In some aspects the dosage is 0.2 mg/kg/day.

In an aspect, disclosed herein are methods comprising the step of administering a composition comprising silvestrol or a silvestrol analog, and a pharmaceutically acceptable carrier, wherein the composition is administered in a dosage and frequency less than 0.5, 0.4, or 0,3 mg/kg/day, wherein the composition further comprises one or more immunomodulatory agents, wherein one or more immunomodulatory agents potentiates an antibody-dependent cell-mediated cytoxicity (“ADCC”) response in the subject. In an aspect, disclosed herein are methods comprising the step of administering a composition comprising silvestrol or a silvestrol analog, and a pharmaceutically acceptable carrier, wherein the silvestrol or silvestrol analog of the composition is administered in a dosage and frequency less than 0.5, 0.4, or 0.3 mg/kg/day, wherein the composition further comprises one or more immunomodulatory agents, wherein one or more immunomodulatory agents potentiates an antibody-dependent cell-mediated cytoxicity (“ADCC”) response in the subject. In some aspects the dosage is 0.2 mg/kg/day.

Disclosed herein are methods comprising the step of administering, to a subject not yet diagnosed with a cancer, a composition comprising silvestrol or a silvestrol analog, and a pharmaceutically acceptable carrier, wherein the composition is administered in a dosage and frequency effective to modulate the immune system in the subject. In an aspect, disclosed herein are methods comprising the step of administering, to a subject not yet diagnosed with a cancer, a composition comprising silvestrol or a silvestrol analog, and a pharmaceutically acceptable carrier, wherein the composition is administered in a dosage and frequency effective to modulate the immune system in the subject, wherein the dosage is less than 0.5, 0.4, or 0.3 mg/kg/day. In some aspects the dosage is 0.2 mg/kg/day.

In an aspect, disclosed herein are methods comprising the step of administering, to a subject not yet diagnosed with a cancer, a composition comprising silvestrol or a silvestrol analog, and a pharmaceutically acceptable carrier, wherein the composition is administered in a dosage and frequency effective to modulate the immune system in the subject. In an aspect, disclosed herein are methods comprising the step of administering, to a subject not yet diagnosed with a cancer, a composition comprising silvestrol or a silvestrol analog, and a pharmaceutically acceptable carrier, wherein the silvestrol or silvestrol analog of the composition is administered in a dosage and frequency effective to modulate the immune system in the subject, wherein the dosage is less than 0.5, 0.4, or 0.3 mg/kg/day. In some aspects the dosage is 0.2 mg/kg/day.

In an aspect, disclosed herein are methods comprising the step of administering, to a subject not yet diagnosed with a cancer, a composition comprising silvestrol or a silvestrol analog, and a pharmaceutically acceptable carrier, wherein the composition is administered in a dosage and frequency effective to modulate the immune system in the subject, wherein the silvestrol or silvestrol analog is silvestrol,

In an aspect, disclosed herein are methods comprising the step of administering, to a subject not yet diagnosed with a cancer, a composition comprising silvestrol or a silvestrol analog, and a pharmaceutically acceptable carrier, wherein the composition is administered in a dosage and frequency effective to modulate the immune system in the subject, wherein the silvestrol or silvestrol analog is a silvestrol analog.

In an aspect, disclosed herein are methods comprising the step of administering, to a subject not yet diagnosed with a cancer, a composition comprising silvestrol or a silvestrol analog, and a pharmaceutically acceptable carrier, wherein the composition is administered in a dosage and frequency effective to modulate the immune system in the subject, wherein the composition further comprises one or more immunomodulatory agents. Examples of immunomodulatory agents include, but are not limited to cytokines, immune system adjuvants, fusion proteins, antibodies, preventive vaccines, therapeutic vaccines, cytokines, biologic fusion constructs, nucleic acid constructs (i.e.: DNA-based vaccines or immune modulatory products), receptor ligands, a cytokine or receptor antagonist, an adoptively transferred cell (NK, T cells, DCs). In some aspects the one or more immunomodulatory agents potentiates an antibody-dependent cell-mediated cytoxicity (“ADCC”) response in the subject.

v) Co-Therapeutic Methods

Disclosed herein are co-therapeutic methods comprising administration of silvestrol or a silvestrol analog, and an agent having a side-effect of immunosuppression. As disclosed herein, administration of silvestrol and silvestrol analogs, unlike many anti-cancer agents, does not affect the humoral immune function or Ig production in a subject and further IFNg production in the subject remains normal. In fact, administration of silvestrol or a silvestrol analog can potentiate virus-specific or tumor-specific cytotoxic T lymphocytes, can virus-specific or tumor-specific T-helper cells, or virus-specific or tumor-specific Th1, Th2, Th17, of Treg cells.

Examples of agents having a side-effect of immunosuppression include, but are not limited to steroids, cytostatics, Antibodies, Immunophilin/modulatory agents. Additional examples of agents having a side-effect of immunosuppression include, but are not limited to nucleoside analogues, alkylating agents, anti metabolites (Methotrexate, folate, azathioprine, mercaptopurine, anti CD3 (OKT3, ATG), CD20 (rituximab), other mAbs that lead to lymphopenia, IVIG, anti compliment antibodies, anti cytokine antibodies, Cyclosproine calcineurin inhibitors, tacrolimis, sirolimus, FK506, mycophenylate, FTY720, IFNs, TNF binding agents, or TGF binding agents.

In an aspect, disclosed herein are co-therapeutic methods comprising administration of silvestrol or a silvestrol analog, and an agent having a side-effect of immunosuppression, wherein the silvestrol analog is a compound of Formula (I) or a salt or prodrug thereof or a compound (including stereoisomers within the dioxanyl group) of formula (i) or a salt or prodrug thereof.

In some aspects, disclosed herein are co-therapeutic methods comprising administration of silvestrol or a silvestrol analog, and an agent having a side-effect of immunosuppression, wherein the silvestrol or a silvestrol analog is administered in an amount in the range of 0.1 to 0.3 micrograms of complex per kg body weight of the subject per administration. In some aspects, disclosed herein are co-therapeutic methods comprising administration of silvestrol or a silvestrol analog, and an agent having a side-effect of immunosuppression, wherein the silvestrol or a silvestrol analog is administered in an amount of 0.2 micrograms of complex per kg body weight of the subject per administration.

In some aspects, disclosed herein are co-therapeutic methods comprising administration of silvestrol or a silvestrol analog, and an agent having a side-effect of immunosuppression, wherein the silvestrol or silvestrol analog or agent having a side-effect of immunosuppression is administered repeatedly to the subject. In some aspects, the repeated administration can be daily, every other day, every third day, every forth day, every fifth day, every sixth day, or once per week. One of skill will be able to determine dosage regime based on the dosage of the compositions given to the subject and the subject him or herself.

In some aspects, the method can be used to treat cancer, a viral infection or a hyperproliferative disorder.

Disclosed herein are co-therapeutic methods comprising administration of silvestrol or a silvestrol analog, and an anti-viral agent. In an aspect, disclosed herein are co-therapeutic methods comprising administration of silvestrol or a silvestrol analog, and an anti-viral agent, wherein the dosage of the anti-viral agent is lower than the standard amount necessary to treat the viral infection in the subject. Examples of anti-viral agents include, but are not limited to anti-herpetic agents, anti-influenza agents, anti-encephalitis agents, anti-hepatitis agents, anti-labyrynthitis agents, anti-lymphoid interstitial pneumonia agents, anti-meningitis agents, anti-orf agents, anti-pneumonia agents, anti-Ramsay Hunt Syndrome Type II agents, anti-SARS agents, anti-shingles agents, anti-Epstein Barr virus agents, anti-EBV agents, anti-HSV agents, anti-HPV agents, adamantane anti-virals, anti-viral combinations, anti-viral interferons, chemokine receptor antagonist, integrase strand transfer inhibitor, miscellaneous anti-virals, neuraminidase inhibitors, NNRTIs, nucleoside reverse transcriptase inhibitors (NRTIs), protease inhibitors, or purine nucleosides.

In some aspects, the dosage of the anti-viral agent is 5%, 10%, 15%, or 20% lower than the standard amount of the anti-viral agent when administered without silvestrol or a silvestrol analog necessary to treat the cancer in the subject.

In an aspect, disclosed herein are co-therapeutic methods comprising administration of silvestrol or a silvestrol analog, and an anti-viral agent, wherein the silvestrol analog is a compound of Formula (I) or a salt or prodrug thereof or a compound (including stereoisomers within the dioxanyl group) of formula (i) or a salt or prodrug thereof.

In an aspect, disclosed herein are co-therapeutic methods comprising administration of silvestrol or a silvestrol analog, wherein the silvestrol or a silvestrol analog is administered in an amount in the range of 0.1 to 0.3 micrograms of complex per kg body weight of the subject per administration. In an aspect, disclosed herein are co-therapeutic methods comprising administration of silvestrol or a silvestrol analog, wherein the silvestrol or a silvestrol analog is administered in an amount of 0.2 micrograms of complex per kg body weight of the subject per administration.

In an aspect, disclosed herein are co-therapeutic methods comprising administration of silvestrol or a silvestrol analog, wherein the silvestrol or silvestrol analog or anti-viral agent having a side—is administered repeatedly to the subject. In some aspects, the repeated administration can be daily, every other day, every third day, every forth day, every fifth day, every sixth day, or once per week. One of skill will be able to determine dosage regime based on the dosage of the compositions given to the subject and the subject him or herself.

In some aspects the anti-viral agent is a vaccine, nucleoside analoge (e.g. AZT, acyclovir, ganciclovir), an anti retroviral agents (e.g. nucleoside RT inhibitors, non nucleoside RT inhibitors, chemokine blockers (CCR5,), integrase inhibitor, compositions in the HAART drug class, siRNA, shRNA, anti-herpetic agents, anti-influenza agents, anti-encephalitis agents, anti-hepatitis agents, anti-labyrynthitis agents, anti-lymphoid interstitial pneumonia agents, anti-meningitis agents, anti-orf agents, anti-pneumonia agents, anti-Ramsay Hunt Syndrome Type II agents, anti-SARS agents, anti-shingles agents, anti-Epstein Barr virus agents, anti-EBV agents, anti-HSV agents, anti-HPV agents, adamantane anti-virals, anti-viral combinations, anti-viral interferons, chemokine receptor antagonist, integrase strand transfer inhibitor, miscellaneous anti-virals, neuraminidase inhibitors, NNRTIs, nucleoside reverse transcriptase inhibitors (NRTIs), protease inhibitors, or purine nucleosides.

In some aspects, the method can be used to treat cancer, a viral, infection or a hyperproliferative disorder.

vi) Methods of Treating Infection

Disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject. In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein the anti-viral agent is a vaccine, nucleoside analoge (e.g. AZT, acyclovir, ganciclovir), an anti retroviral agents (e.g. nucleoside RT inhibitors, non nucleoside RT inhibitors, chemokine blockers (CCR5,), an integrase inhibitor, compositions in the HAART drug class, siRNA, shRNA, anti-herpetic agents, anti-influenza agents, anti-encephalitis agents, anti-hepatitis agents, anti-labyrynthitis agents, anti-lymphoid interstitial pneumonia agents, anti-meningitis agents, anti-orf agents, anti-pneumonia agents, anti-Ramsay Hunt Syndrome Type II agents, anti-SARS agents, anti-shingles agents, anti-Epstein Barr virus agents, anti-EBV agents, anti-HSV agents, anti-HPV agents, adamantane anti-virals, anti-viral combinations, anti-viral interferons, chemokine receptor antagonist, integrase strand transfer inhibitor, miscellaneous neuraminidase inhibitors, NNRTIs, nucleoside reverse transcriptase inhibitors (NRTIs), protease inhibitors, or purine nucleosides.

In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein the dosage of the anti-viral agent is lower than the standard amount necessary to treat the viral-infection in the subject. In some aspects, the dosage of the viral-infection is 5%, 10%, 15%, or 20% lower than the standard amount of the viral-infection when administered without silvestrol or a silvestrol analog necessary to treat the viral-infection in the subject.

In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein the silvestrol analog is a compound of Formula (I) or a salt or prodrug thereof or a compound (including stereoisomers within the dioxanyl group) of formula (i) or a salt or prodrug thereof.

In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein the composition is administered in an amount in the range of 0.1 to 200 micrograms of complex per kg body weight of the subject per administration. In an aspect, disclosed herein are treatment methods comprising the step of administering, to a subject diagnosed with a viral-infection, a composition comprising: (a) silvestrol or a silvestrol analog; (b) an anti-viral agent; and (c) it pharmaceutically acceptable carrier, wherein the composition is administered in a dosage and frequency synergistically effective to treat the viral-infection in the subject, wherein the silvestrol or a silvestrol analog of the composition is administered in an amount in the range of 0.1 to 0.3 micrograms of complex per kg body weight of the subject per administration. In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein the silvestrol or a silvestrol analog of the composition is administered in an amount of 0.2 micrograms of complex per kg body weight of the subject per administration.

In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein the composition is administered repeatedly to the subject. In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein the subject is a mammal such as a human. In some aspects, the repeated administration can be daily, every other day, every third day, every forth day, every fifth day, every sixth day, or once per week. One of skill will be able to determine dosage regime based on the dosage of the compositions, given to the subject and the subject him or herself.

In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein the subject has been diagnosed with a need for modulating the immune system prior to the administering step.

In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, further comprising the step of identifying a subject in need of modulating the immune system.

In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein the viral-infection is a systemic, localized, acute, chronic, recurrent, asymptomatic viral infection.

In an aspect, disclosed herein are treatment methods comprising the step of administering, to a subject diagnosed with a viral-infection, a composition comprising: (a) silvestrol or a silvestrol analog; (b) an anti-viral agent; and (c) a pharmaceutically acceptable carder, wherein the composition is administered in a dosage and frequency synergistically effective to treat the viral-infection in the subject, wherein humoral immune function in the subject is unaffected, wherein Ig production in the subject is unaffected, wherein IFNg production in the subject remains normal, wherein the composition potentiates virus-specific or tumor-specific cytotoxic T lymphocytes, wherein the composition potentiates virus-specific or tumor-specific T-helper cells, wherein the composition potentiates virus-specific or tumor-specific T-helper cells, or wherein the composition potentiates virus-specific or tumor-specific Th1 , Th2, Th17, Treg cells.

In an aspect, disclosed herein are treatment methods comprising the step of administering, to a subject diagnosed with a viral-infection, a composition comprising: (a) silvestrol or a silvestrol analog, (b) an anti-viral agent; and (c) a pharmaceutically acceptable carrier, wherein the composition is administered in a dosage and frequency synergistically effective to treat the viral-infection in the subject, wherein the composition further comprises one or more immunomodulatory agents.

In an aspect, disclosed herein are treatment methods comprising the step of administering, to a subject diagnosed with a viral infection, a composition comprising: (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein the one or more immunomodulatory agents is a cytokine, immune system adjuvant, fusion protein, an antibody, a preventive vaccine, a therapeutic vaccine, a cytokine, a biologic fusion construct, a nucleic acid constructs (i.e., DNA-based vaccines or immune modulatory products), a receptor ligand, a cytokine or receptor antagonist, or an adoptively transferred cell (NK, T cells, DCs).

In an aspect, disclosed herein are treatment methods comprising the step of administering, to a subject diagnosed with a viral-infection, a composition comprising; (a) silvestrol or a silvestrol analog; (b) an anti-viral agent; and (c) a pharmaceutically acceptable carrier, wherein the composition is administered in a dosage and frequency synergistically effective to treat the viral-infection in the subject, wherein one or more immunomodulatory agents potentiates an antibody-dependent cell-mediated cytoxicity (“ADCC”) response in the subject.

In some aspects the anti-viral agent is a vaccine, nucleoside analoge (e.g. AZT, acyclovir, ganciclovir), an anti retroviral agents (e.g. nucleoside RT inhibitors, non nucleoside RT inhibitors, chemokine blockers (CCR5,), an integrase inhibitor, compositions in the HAART drug class, siRNA, shRNA, anti-herpetic agents, anti-influenza agents, anti-encephalitis agents, anti-hepatitis agents, anti-labyrynthitis agents, anti-lymphoid interstitial pneumonia agents, anti-meningitis agents, anti-orf agents, anti-pneumonia agents, anti-Ramsay Hunt Syndrome Type II agents, anti-SARS agents, anti-shingles agents, anti-Epstein Barr virus agents, anti -EBV agents, anti-HSV agents, anti-HPV agents, adamantane anti-viral combinations, anti-viral interferons, chemokine receptor antagonist, integrase strand transfer inhibitor, miscellaneous anti-virals, neuraminidase inhibitors, NNRTIs, nucleoside reverse transcriptase inhibitors (NRTIs), protease inhibitors, or purine nucleosides.

vii) Treatment of Disease States or Conditions Associated with Cellular Hyperproliferation

In addition to treating cancer or viral infections, the methods described herein can be used to treat one or more disease state or conditions associated with cellular hyperproliferation. Examples of disease state or conditions associated with cellular hyperproliferation include, but are not limited to atherosclerosis, restinosis, rheumatoid arthritis, osteoarthritis, inflammatory arthritis, psoriasis, peridontal disease or virally induced cellular hyperproliferation.

A cancer or cancerous conditions can be a tissue that comprises neoplastic cells, exhibits an abnormal growth of cells and/or hyperproliferative cells. As used herein, the term “neoplastic” means an abnormal growth of a cell or tissue (e.g., a tumor or non-solid hyper proliferative cellular activity) which may be benign or cancerous. As used herein, “abnormal growth of cells” and/or “hyperproliferative cells” are meant to refer to cell growth independent of normal regulatory mechanisms (e.g., loss of contact inhibition), including the abnormal growth of benign and malignant cells or other neoplastic diseases. As used herein, the term “tumor” includes neoplasms that are identifiable through clinical screening or diagnostic procedures including, but not limited to, palpation, biopsy, cell proliferation index, endoscopy, mammography, digital mammography, ultrasonography, computed tomography (CT), magnetic resonance imaging (MRI), positron emission tomography (PET), radiography, radionuclide evaluation, CT- or MRI-guided aspiration cytology, and imaging-guided needle biopsy, among others. Such diagnostic techniques are well known to those skilled in the art and are described in Holland, et al., Cancer Medicine, 4th Ed., Vol. One, Williams & Wilkins, Baltimore, Md. (1997).

The disclosed compounds can be used as single agents or in combination with one or mare other drugs in the treatment, prevention, control, amelioration or reduction of risk of the aforementioned diseases and conditions for which the disclosed compounds of or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

The compositions and methods of the present invention can further comprise other therapeutically active as noted herein which are usually applied in the treatment of the above mentioned disorders or pathological conditions.

In a further aspect, the compound administered is a disclosed compound or a product of a disclosed method of making a compound.

In a further aspect, the animal is a mammal. In a yet further aspect, the mammal is primate. In a still further aspect, the mammal is a human. In an even further aspect, the human is a patient. In a further aspect, the animal is a domesticated animal. In a still further aspect, the domesticated animal is a domesticated fish, domesticated crustacean, or domesticated mollusk. In a yet further aspect, the domesticated animal is poultry. In an even further aspect, the poultry is selected from chicken, turkey, duck, and goose. In a still, further aspect, the domesticated animal is livestock. In a yet further aspect, the livestock animal is selected from pig, cow, horse, goat, bison, and sheep.

In a further aspect, the effective amount is a therapeutically effective amount. In a still further aspect, the effective amount is a prophylactically effective amount. In a yet further aspect, cancer or a viral infection is prevented by administration of the compound.

In a further aspect, the compound is administered in an effective amount. In a yet further aspect, the effective amount is a therapeutically effective amount. In a still further aspect, the effective amount is a prophylactically effective amount.

viii) Manufacture of a Medicament

In one aspect, the invention relates to a method for the manufacture of a medicament for treating or inhibiting cancer, a viral infection, or a disease state or condition associated with cellular hyperproliferation in a mammal comprising combining a therapeutically effective amount of one or more of the disclosed compounds or product of a disclosed methods with a pharmaceutically acceptable carrier or diluent.

In a further aspect, the medicament modulates the immune system or a cell of the immune system. In a still further aspect, the medicament inhibits viral gene expression.

ix) Use of Compositions

In an aspect, the invention relates to the use of silvestrol or a silvestrol analog for modulating the immune system of a subject. In an aspect, the invention relates to the use of silvestrol or a silvestrol analog for modulating the immune system of a subject, wherein the subject has been diagnosed with cancer.

In an aspect, the invention relates to the use of silvestrol or a silvestrol analog for modulating the immune system of a subject, wherein the subject has not yet been diagnosed with canker.

In an aspect, the invention relates to the use of silvestrol or a silvestrol analog for modulating the immune system of a subject, wherein the subject has been diagnosed with a viral infection.

In an aspect, the invention relates to the use of silvestrol or a silvestrol analog for modulating the immune system of a subject, wherein the subject has not yet been diagnosed with a viral infection.

In an aspect, the invention relates to the use of silvestrol or a silvestrol analog for modulating the activity of an immune cell in a subject.

In an aspect, the invention relates to the use of silvestrol or a silvestrol analog for modulating the activity of an immune cell in a subject, wherein the subject has been diagnosed with cancer.

In an aspect, the invention relates to the use of silvestrol or a silvestrol analog for modulating the activity of an immune cell in a subject, wherein the subject has not yet been diagnosed with cancer.

In an aspect, the invention relates to the use of silvestrol or a silvestrol analog for modulating the activity of an immune cell in a subject, wherein the subject has been diagnosed with a viral infection.

In an aspect, the invention relates to the use of silvestrol or a silvestrol analog for modulating the activity of an immune cell in a subject, wherein the subject has not yet been diagnosed with a viral infection.

In an aspect, the invention relates to the use of silvestrol or a silvestrol analog for modulating viral gene expression in a subject.

In an aspect, the invention relates to the use of silvestrol or a silvestrol analog for modulating viral gene expression in a subject, wherein the subject has been diagnosed with cancer.

In an aspect, the invention relates to the use of silvestrol or a silvestrol analog for modulating viral gene expression in a subject, wherein the subject has not yet been diagnosed with cancer.

In an aspect, the invention relates to the use of silvestrol or a silvestrol analog for modulating viral gene expression in a subject, wherein the subject has been diagnosed with a viral infection.

In an aspect, the invention relates to the use of silvestrol or a silvestrol analog for modulating viral gene expression in a subject, wherein the subject has not yet been diagnosed with a viral infection.

In an aspect, the invention relates to the use of silvestrol or a silvestrol analog in combination with one or more immunomodulatory agents for modulating the activity of an immune cell in a subject.

In an aspect, the invention relates to the use of silvestrol or a silvestrol analog in combination with one or more immunomodulatory agents for modulating the activity of an immune cell in a subject, wherein the subject has been diagnosed with cancer.

In an aspect, the invention relates to the use of silvestrol or a silvestrol analog in combination with one or more immunomodulatory agents for modulating the activity of an immune cell in a subject, wherein the subject has not yet been diagnosed with cancer.

In an aspect, the invention relates to the use of silvestrol or a silvestrol analog in combination with one or more immunomodulatory agents for modulating the activity of an immune cell in a subject, wherein the subject has been diagnosed with a viral infection.

In an aspect, the invention relates to the use of silvestrol or a silvestrol analog in combination with one or more immunomodulatory agents for modulating the activity of an immune cell in a subject, wherein the subject has not yet been diagnosed with a viral infection.

In an aspect, the invention relates to the use of silvestrol or a silvestrol analog in combination with one or more anti-cancer agents for treating cancer in a subject.

In an aspect, the invention relates to the use of silvestrol or a silvestrol analog in combination with one or more anti-cancer agents for treating cancer in a subject, wherein the subject has been diagnosed with cancer.

In an aspect, the invention relates to the use of silvestrol or silvestrol analog in combination with one or more anti-cancer agents for treating cancer in a subject, wherein the subject has not yet been diagnosed with cancer.

In an aspect, the invention relates to the use of silvestrol or a silvestrol analog in combination with one or more anti-cancer agents for treating cancer in a subject, wherein the subject has been diagnosed with a viral infection.

In an aspect, the invention relates to the use of silvestrol or a silvestrol analog in combination with one or more anti-cancer agents for treating cancer in a subject, wherein the subject has not yet been diagnosed with a viral infection.

In an aspect, the invention relates to the use of silvestrol or a silvestrol analog in combination with one or more anti-viral agents for treating cancer in a subject.

In an aspect, the invention relates to the use of silvestrol or a silvestrol analog in combination with one or more anti-viral agents for treating cancer in a subject, wherein the subject has been diagnosed with cancer.

In an aspect, the invention relates to the use of silvestrol or a silvestrol analog in combination with one or more anti-viral agents for treating cancer in a subject, wherein the subject has not yet been diagnosed with cancer.

In an aspect, the invention relates to the use of silvestrol or a silvestrol analog in combination with one or more anti-viral agents for treating cancer in a subject, wherein the subject has been diagnosed with a viral infection.

In an aspect, the invention relates to the use of silvestrol or a silvestrol analog in combination with one or more anti-viral agents for treating cancer in a subject, wherein the subject has not yet been diagnosed with a viral infection.

In an aspect, the invention relates to the use of silvestrol or a silvestrol analog in combination with one or more anti-viral agents for treating a viral infection in a subject.

In an aspect, the invention relates to the use of silvestrol or a silvestrol analog in combination with one or more anti-viral agents for treating a viral infection in a subject, wherein the subject has been diagnosed with cancer.

In an aspect, the invention relates to the use of silvestrol or a silvestrol analog in combination with one or more anti-viral agents for treating a viral infection in a subject, wherein the subject has not yet been diagnosed with cancer.

In an aspect, the invention relates to the use of silvestrol or a silvestrol analog in combination with one or more anti-viral agents for treating a viral infection in a subject, wherein the subject has been diagnosed with a viral infection.

In an aspect, the invention relates to the use of silvestrol or a silvestrol analog in combination with one or more anti-viral agents for treating a viral infection in a subject, wherein the subject has not yet been diagnosed with a viral infection.

In a further aspect, a use is the treatment of a mammal. In a yet further aspect, the mammal is a human. In a still further aspect, the human is a patient. In a yet further aspect, a use is administration of the compound to a mammal to treat or prevent cancer, viral infection, or a disease state or conditions associated with cellular hyperproliferation. In a further aspect, the mammal is a human.

In a further aspect, a use is administration of the compound in an effective amount. In a yet farther aspect, the effective amount is a therapeutically effective amount. In a still further aspect, the effective amount is a prophylactically effective amount. In a still further aspect, prior to use the subject in need of treatment is identified. In a yet further aspect, prior to use the mammal in need of prevention is identified. In an even further aspect, the mammal has been diagnosed with a need for treatment of the disorder or disease prior to the administering step.

In some aspects the anti-viral agent is a vaccine, nucleoside analoge (e.g. AZT, acyclovir, ganciclovir), an anti retroviral agents (e.g. nucleoside RT inhibitors, non nucleoside RT inhibitors, chemokine blockers (CCR5,), integrase inhibitor, compositions in the HAART drug class, siRNA, shRNA, anti-herpetic agents, anti-influenza agents, anti-encephalitis agents, anti-hepatitis agents, anti-labyrynthitis agents, anti-lymphoid interstitial pneumonia agents, anti-meningitis agents, and-orf agents, anti-pneumonia agents, anti-Ramsay Hunt Syndrome Type II agents, anti-SARS agents, anti-shingles agents, anti-Epstein Barr virus agents, anti-EBV agents, anti-HSV agents, anti-HPV agents, adamantane anti-virals, anti-viral combinations, anti-viral interferons, chemokine receptor antagonist, integrase strand transfer inhibitor, miscellaneous anti-virals, neuraminidase inhibitors, NNRTIs, nucleoside reverse transcriptase inhibitors (NRTIs), protease inhibitors, or purine nucleosides.

x) Kits

Disclosed herein are kits comprising at least silvestrol or a silvestrol analog or a pharmaceutically acceptable salt thereof, and one or more of: an anti-cancer agent, an immune modulatory agent, an agent having a side-effect of immunosuppression, an anti-viral agent, an agent known to modulate the immune system; at least one agent known to modulate the activity of an immune cell; at least one agent known to modulate viral gene expression; at least one agent known to treat cancer; at least one agent known to treat one or more symptoms of cancer; at least one agent known to treat a disease state or conditions associated with cellular hyperproliferation; instructions for modulating the immune system in a subject; instructions for modulating the activity of an immune cell in a subject; instructions for treating a disease state or conditions associated with cellular hyperproliferation; instructions for treating a cancer in a subject, instructions for vaccinating a subject not yet diagnosed with cancer, instructions for administering a composition comprising silvestrol or a silvestrol analog and a pharmaceutically acceptable carrier to a subject, instructions for administration of silvestrol or a silvestrol analog and an agent having a side-effect of immunosuppression to a subject, or instructions for administration of silvestrol or a silvestrol as and an anti-viral agent to a subject.

In an aspect, disclosed herein are kits comprising at least silvestrol or a silvestrol analog or a pharmaceutically acceptable salt thereof, and one or more of: an and-cancer agent, an immune modulatory agent, an agent having a side-effect of immunosuppression, an anti-viral agent, an agent known to modulate the immune system; at least one agent known to modulate the activity of an immune cell; at least one agent known to modulate viral gene expression; at least one agent known to treat cancer; at least one agent known to treat one or more symptoms of cancer; at least one agent known to treat a disease state or conditions associated with cellular hyperproliferation; instructions for modulating the immune system in a subject; instructions for modulating the activity of an immune cell in a subject; instructions for treating a disease state or conditions associated with cellular hyperproliferation; instructions for treating a cancer in a subject, instructions for vaccinating a subject not yet diagnosed with cancer, instructions for administering a composition comprising silvestrol or a silvestrol analog and a pharmaceutically acceptable carrier to a subject, instructions far administration of silvestrol or a silvestrol analog and an agent having a side-effect of immunosuppression to a subject, or instructions for administration of silvestrol or a silvestrol analog and an anti-viral agent to a subject, wherein the at least one compound and the at least one agent are co-formulated.

In an aspect, disclosed herein are kits comprising at least silvestrol or a silvestrol analog or a pharmaceutically acceptable salt thereof, and one or more of; an anti-cancer agent, an immune modulatory agent, an agent having a side-effect of immunosuppression, an anti-viral agent, an agent known to modulate the immune system; at least one agent known to modulate the activity of an immune cell; at least one agent known to modulate viral gene expression; at least one agent known to treat cancer; at least one agent known to treat one or more symptoms of cancer; at least one agent known to treat a disease state or conditions associated with cellular hyperproliferation; instructions for modulating the immune system in a subject; instructions for modulating the activity of an immune cell in a subject; instructions for treating a disease state or conditions associated with cellular hyperproliferation; instructions for treating a cancer in a subject, instructions for vaccinating a subject not yet diagnosed with cancer, instructions for administering a composition comprising silvestrol or a silvestrol analog and a pharmaceutically acceptable carrier to a subject, instructions for administration of silvestrol or a silvestrol analog and an agent having a side-effect of immunosuppression to a subject, or instructions for administration of silvestrol or a silvestrol analog and an anti-viral agent to a subject, wherein the at least one compound and the at least one agent are co-packaged.

Disclosed herein are kits comprising at least silvestrol or a silvestrol analog or a pharmaceutically acceptable salt thereof and an anti-cancer agent. In an aspect, disclosed are kits comprising at least silvestrol or a silvestrol analog or a pharmaceutically acceptable salt thereof and an anti-cancer agent wherein the anti-cancer agent is an antibody, a preventive vaccine, therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, the silvestrol or a silvestrol analog and anti-cancer agent are coformulated. In a further aspect, the silvestrol or a silvestrol analog and anti-cancer agent are copackaged. In an aspect, the kit further comprises instructions for treatment of cancer.

Disclosed herein are kits comprising at least silvestrol or a silvestrol analog or a pharmaceutically acceptable salt thereof and an immune modulatory agent. In an aspect, disclosed are kits comprising at least silvestrol or a silvestrol analog or a pharmaceutically acceptable salt thereof and an immune modulatory agent, wherein the immune modulatory agent is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, the silvestrol or silvestrol analog and immune modulatory agent are coformulated. In a further aspect, the silvestrol or a silvestrol analog and immune modulatory agent are copackaged. In an aspect, the kit further comprises instructions for modulating the immune system.

Disclosed herein are kits comprising at least silvestrol or a silvestrol analog or a pharmaceutically acceptable salt thereof and an agent having a side-effect of immunosuppression. In an aspect, disclosed herein are kits comprising at least silvestrol or a silvestrol analog or a pharmaceutically acceptable salt thereof and an agent having a side-effect of immunosuppression, wherein the agent having a side-effect of immunosuppression is a steroid, cytostatic, antibody, or an immunophilin/modulatory agent. In an aspect, disclosed herein are kits comprising at least silvestrol or a silvestrol analog or a pharmaceutically acceptable salt thereof and an agent having a side-effect of immunosuppression, wherein the agent having a side-effect of immunosuppression is a nucleoside analogue, alkylating agent, anti metabolite (Methotrexate, folate, azathioprine, mercaptopurine, anti CD3 (OKT3, ATG), CD20 (rituximab), other mAbs that lead to lymphopenia, IVIG, anti compliment antibody, anti cytokine antibodies, Cyclosproine, calcineurin inhibitors, tacrolimis, sirolimus, FK506, mycophenylate, FTY720, IFNs, TNF binding agents, or TGF binding agents.

In an aspect, the silvestrol or silvestrol analog and agent having a side-effect of immunosuppression are coformulated. In a further aspect, the silvestrol or a silvestrol analog and agent having a side-effect of immunosuppression are copackaged. In an aspect, the kit further comprises instructions for modulating the immune system or suppressing the immune system or treating cancer.

Disclosed herein are kits comprising at least silvestrol or a silvestrol analog or a pharmaceutically acceptable salt thereof and an anti-viral agent. In an aspect, disclosed herein are kits comprising at least silvestrol or a silvestrol analog or a pharmaceutically acceptable salt thereof and an anti-viral agent, wherein the anti-viral agent is a nucleoside analoge (e.g. AZT, acyclovir, ganciclovir), an anti retroviral agents (e.g. nucleoside RT inhibitors, non nucleoside RT inhibitors, chemokine blockers (CCR5,), an integrase inhibitor, compositions in the HAART drug class, siRNA or shRNA.

In an aspect, the silvestrol or silvestrol analog and anti-viral agent are coformulated. In a further aspect, the silvestrol or silvestrol analog and anti-viral agent are copackaged. In an aspect, the kit further comprises instructions for modulating the immune system or suppressing the immune system treating cancer, or treating a viral infection.

Disclosed herein are kits comprising at least silvestrol or a silvestrol analog or a pharmaceutically acceptable salt thereof and an agent known to modulate the immune system. In an aspect, disclosed herein are kits comprising at least silvestrol or a silvestrol analog or a pharmaceutically acceptable salt thereof and an agent known to modulate the immune system, wherein the an agent known to modulate the immune system is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, the silvestrol or silvestrol analog and agent known to modulate the immune system are coformulated. In a further aspect, the silvestrol or silvestrol analog and agent known to modulate the immune system are copackaged. In an aspect, the kit further comprises instructions for modulating the immune system or suppressing the immune system treating cancer, or treating a viral infection.

Disclosed herein are kits comprising at least silvestrol or a silvestrol analog or a pharmaceutically acceptable salt thereof and at least one agent known to modulate the activity of an immune cell. In an aspect, disclosed herein are kits comprising at least silvestrol or a silvestrol analog or a pharmaceutically acceptable salt thereof and at least one agent known to modulate the activity of an immune cell, wherein the agent known to modulate the activity of an immune cell is a cytokine, immune system adjuvant, fusion protein, an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, the silvestrol or silvestrol analog agent known to modulate the immune system are coformulated. In a further aspect, the silvestrol or silvestrol analog and agent known to modulate the immune system are copackaged. In an aspect, the kit further comprises instructions for modulating the immune system or suppressing the immune system treating cancer, or treating a viral infection.

Disclosed herein are kits comprising at least silvestrol or a silvestrol analog or a pharmaceutically acceptable salt thereof and at least one agent known to modulate viral gene expression. In an aspect, disclosed herein are kits comprising at least silvestrol or a silvestrol analog or a pharmaceutically acceptable salt thereof and at least one agent known to modulate viral gene expression, wherein the at least one agent known to modulate viral gene expression is anti-herpetic agents, anti-influenza agents, anti-encephalitis agents, anti-hepatitis agents, anti-labyrynthitis agents, anti-lymphoid interstitial pneumonia agents, anti-meningitis agents, anti-orf agents, anti-pneumonia agents, anti-Ramsay Hunt Syndrome Type II agents, anti-SARS agents, anti-shingles agents, anti-Epstein Barr virus agents, anti-EBV agents, anti-HSV agents, anti-HPV agents, adamantane anti-virals, anti-viral combinations, anti-viral interferons, chemokine receptor antagonist, integrase strand transfer inhibitor, miscellaneous anti-virals, neuraminidase inhibitors, NNRTIs, nucleoside reverse transcriptase inhibitors (NRTIs), protease inhibitors, or purine nucleosides. In an aspect, the silvestrol or silvestrol analog and agent known to modulate viral gene expression are coformulated. In a further aspect, the silvestrol or silvestrol analog and agent known to modulate viral gene expression are copackaged. In an aspect, the kit further comprises instructions for modulating the immune system or suppressing the immune system treating cancer, or treating a viral infection.

Disclosed herein are kits comprising at least silvestrol or a silvestrol analog or a pharmaceutically acceptable salt thereof and at least one agent known to treat cancer. In an aspect, disclosed herein are kits comprising at least silvestrol or a silvestrol analog or a pharmaceutically acceptable salt thereof and at least one agent known to treat cancer, wherein the at least one agent known to treat cancer is an antibody, a preventive vaccine, a therapeutic vaccine, cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, the silvestrol or silvestrol analog and agent known to treat cancer are coformulated. In a further aspect, the silvestrol or silvestrol analog and agent known to treat cancer are copackaged. In an aspect, the kit further comprises instructions for modulating the immune system or suppressing the immune system treating cancer, or treating a viral infection.

Disclosed herein are kits comprising at least silvestrol or a silvestrol analog or a pharmaceutically acceptable salt thereof and at least one agent known to treat one or more symptoms of cancer. In an aspect, disclosed herein are kits comprising at least silvestrol or a silvestrol analog or a pharmaceutically acceptable salt thereof and at least one agent known to treat one or more symptoms of cancer, wherein the at least one agent known to treat one or more symptoms of cancer is an antibody, a preventive vaccine, a therapeutic vaccine, a cytokine, a biologic fusion construct, a nucleic acid constructs (i.e.: DNA-based vaccines or immune modulatory products), a receptor ligand, a cytokine or receptor antagonist, or an adoptively transferred cell (NK, T cells, DCs). In an aspect, the silvestrol or silvestrol analog and agent known to treat one or more symptoms of cancer are coformulated. In a further aspect, the silvestrol or silvestrol analog and agent known to treat one or more symptoms of cancer are copackaged. In an aspect, the kit further comprises instructions for modulating the immune system or suppressing the immune system treating cancer, or treating a viral infection.

Disclosed herein are kits comprising at least silvestrol or a silvestrol analog or a pharmaceutically acceptable salt thereof and at least one agent known to treat a disease state or conditions associated with cellular hyperproliferation. In an aspect, disclosed herein are kits comprising at least silvestrol or a silvestrol analog or a pharmaceutically acceptable salt thereof and at least one agent known to treat a disease state or conditions associated with cellular hyperproliferation, wherein the at least one agent known to treat a disease state or conditions associated with cellular hyperproliferation is atherosclerosis, restinosis, rheumatoid arthritis, osteoarthritis, inflammatory arthritis, psoriasis, peridontal disease or virally induced cellular hyperproliferation. In an aspect, the silvestrol or silvestrol analog and agent known to treat a disease state or conditions associated with cellular hyperproliferation are coformulated. In a further aspect, the silvestrol or silvestrol analog and agent known to treat a disease state or conditions associated with cellular hyperproliferation are copackaged. In an aspect, the kit further comprises instructions for modulating the immune system or suppressing the immune system treating cancer, or treating a viral infection.

In a further aspect, the kit comprises a disclosed compound or a product of a disclosed methods.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

In some aspects the anti-viral agent is a vaccine, nucleoside analog (e.g. AZT, acyclovir, ganciclovir), an anti retroviral agents (e.g. nucleoside RT inhibitors, non nucleoside RT inhibitors, chemokine blockers (CCR5,), an integrase inhibitor, compositions in the HAART drug class, siRNA, shRNA, anti-herpetic agents, anti-influenza agents, anti-encephalitis agents, anti-hepatitis agents, anti-labyrynthitis agents, anti-lymphoid interstitial pneumonia agents, anti-meningitis agents, anti-orf agents, anti-pneumonia agents, anti-Ramsay Hunt Syndrome Type II agents, anti-SARS agents, anti-shingles agents, anti-Epstein Barr virus agents, anti-EBV agents, anti-HSV agents, anti-HPV agents, adamantane anti-virals, anti-viral combinations, anti-viral interferons, chemokine receptor antagonist, integrase strand transfer inhibitor, miscellaneous anti-vitals, neuraminidase inhibitors, NNRTIs, nucleoside reverse transcriptase inhibitors (NRTIs), protease inhibitors, or purine nucleosides.

It is contemplated that the disclosed kits can be used in connection with the disclosed methods of making, the disclosed methods of using, and/or the disclosed compositions.

xi) Non-Medical Uses

Also provided are the uses of the disclosed compounds and products as pharmacological tools in the development and standardization of in vitro and in vivo test systems for the evaluation of the effects of modulators of cancer, viral infection or the immune system or inhibitors of cancer, viral infection or the immune system related activity in laboratory animals such as cats, dogs, rabbits, monkeys, rats and mice, as part of the search for new therapeutic agents of cancer, viral infection or the immune system.

### E. Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing front the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in °C. or is at ambient temperature, and pressure is at or near atmospheric.

### EXAMPLES

### A. Example 1

### Immunomodulatory Properties of Silvestrol

i) Silvestrol Discovery and Characterization

Cyclopenta[b]benzofuran constituents of plants from the genus Aglaia were first discovered in 1982, and have been of considerable interest since one such molecule, rocaglamide, was found to exhibit anti-leukemic activity in the P388 lymphocytic leukemic murine model [King 1982; Rose 1988]. The anti-proliferative activity of members of the cyclopenta[b]benzofuran class have been demonstrated using human tumor cell lines [Bohnenstengel 1999; Bohnenstengel 1999; Su 2006] (reviewed in Kim 2006) and primary human tumor cells [Zhu 2007]. Silvestrol, a member of this class, was identified from tropical plants by activity-guided chromatographic fractionation. The identification of cytotoxic extracts from Aglaia foveolata led to the purification of silvestrol as well as (+)-episilvestrol, its C-5′″ S epimer at the diol side chain of the dioxanyl ring. Using detailed NMR studies and single-crystal X-ray diffraction, the structure and absolute configuration of silvestrol was then characterized [Hwang et al].

Silvestrol is a unique member of the cyclopenta[b]benzofuran class, and bears a bulky dioxanyl group unprecedented in nature. Preliminary structure-activity relationship studies indicate that the dioxanyl side chain is important for cytotoxicity, as silvestrol is much more potent than rocaglamide in vitro, and acetylation of the two dioxanyloxy hydroxyl groups causes a ten-fold reduction in potency [Hwang 2004]. Because of its unique structure and high potency, silvestrol has attracted the attention of synthetic organic chemists. The total syntheses of (−)-silvestrol has been demonstrated by El Sous 2007 and Gerard 2007, which confirmed the structure and stereochemistry reported earlier. The synthesis of silvestrol has been refined by Adams 2009, which refinement generated additional bioactive derivatives [Adams 2009]. These studies indicate that stereochemistry of the cyclopentabenzofuran backbone was critical for the anti-cancer activity of silvestrol. Structure-activity relationship studies by Cencic 2009 confirmed the importance of both the cyclopenta[b]benzofuran core and side chain for optimal potency. While silvestrol was originally isolated from fruits and twigs of A. foveolata, work shows it can be successfully extracted from the leaves and stems of this plant [Salim 2004]. The occurrence of this compound in several plant parts including leaves enables silvestrol to be produced in large quantities as a “renewable resource” that will not sacrifice the plant origin.

Work with members of the same class show that their targets can include inhibition of nucleotide and/or protein synthesis or inhibition of transcription factors NF-κB [Baumann 2005] or NF-AT [Baumann 2005]. Zhu et al. described a potential mechanism for rocaglamide-mediated cytotoxicity in which activation of p38 MAPK, together with suppression of ERK, leads apoptosis involving activation of caspases 8 and 9. Specific to silvestrol, Swanson et al. showed that this agent produces a p53-independent cell cycle blockade at the G2/M checkpoint in the LNCaP human prostate cancer cell line [Mi. 2006], consistent with an effect on cyclins. Silvestrol activates an unusual pathway of apoptosis in LNCaP cells, as indicated by the involvement of caspases 2, 9 and 10, but not 3 and 7 [Kim 2007]. It was also shown that silvestrol treatment of B leukemia cells results in loss of the pro-survival protein Mcl-1 by selective inhibition of translation [Lucas 2009]. Together, it is evident that this family of agents has multiple targets that vary not only by agent but also by cell type. The inhibition of translation initiation has been defined as a key mechanism in the cytotoxic activity of silvestrol [Bordeleau 2008; Cencic 2009].

ii) Epstein Barr Virus (EBV)-Associated Models to Study Lymphomagenesis and Immunity.

(1) EBV Overview:

Epstein-Barr virus (EBV) is an oncogenic human herpes virus that infects more than 90% of people worldwide, EBV infection persists for the lifetime of the host and frequently reactivates throughout an individual's lifetime. EBV has the capacity to transform epithelial cells and lymphocytes, and is associated with a broad spectrum of pathology that collectively leads to over 100,000 new diagnoses of cancer each year worldwide. EBV is believed to be the etiologic agent in nasopharyngeal carcinoma (NPC), gastric carcinoma, and several lymphoproliferative disorders including Hodgkin's and non-Hodgkin lymphomas. EBV-related lymphomas often manifest as opportunistic malignancies that arise in patients with profound immune deficiency and, more recently, have been noted with increasing frequency in elderly patient populations. EBV-associated malignancies often present with extra nodal involvement and are associated with a poor prognosis While combination chemotherapy, radiotherapy and surgery are often used to manage patients with these disorders, such treatments lead to prolonged immune suppression leaving the patient vulnerable to persistent EBV reactivation and relapsed, EBV-driven disease.

iii) Silvestrol and B Cell Malignancies.

Silvestrol is a potent inhibitor of the initiation step of translation, and shows anti-tumor efficacy in several preclinical tumor models. The translation inhibitory activity of silvestrol led to an early depletion of the anti-apoptotic protein Mcl-1 in chronic lymphocytic leukemia cells, with concomitant mitochondrial depolarization and caspase-dependent apoptosis. Its increased potency against B cells relative to T cells both in vitro and in vivo, and significantly prolonged survival in a mouse model of acute lymphoblastic leukemia.

iv) Direct Activity of Silvestrol Against EBV-Driven Malignancy.

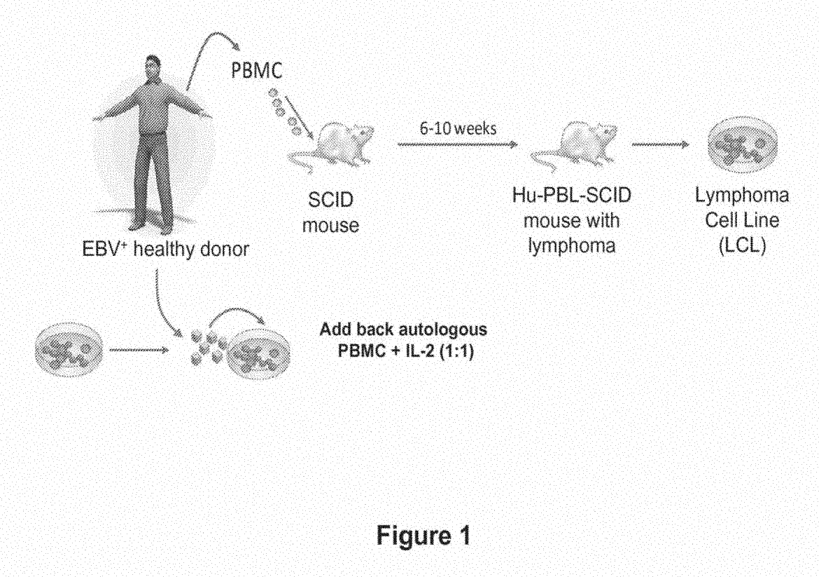

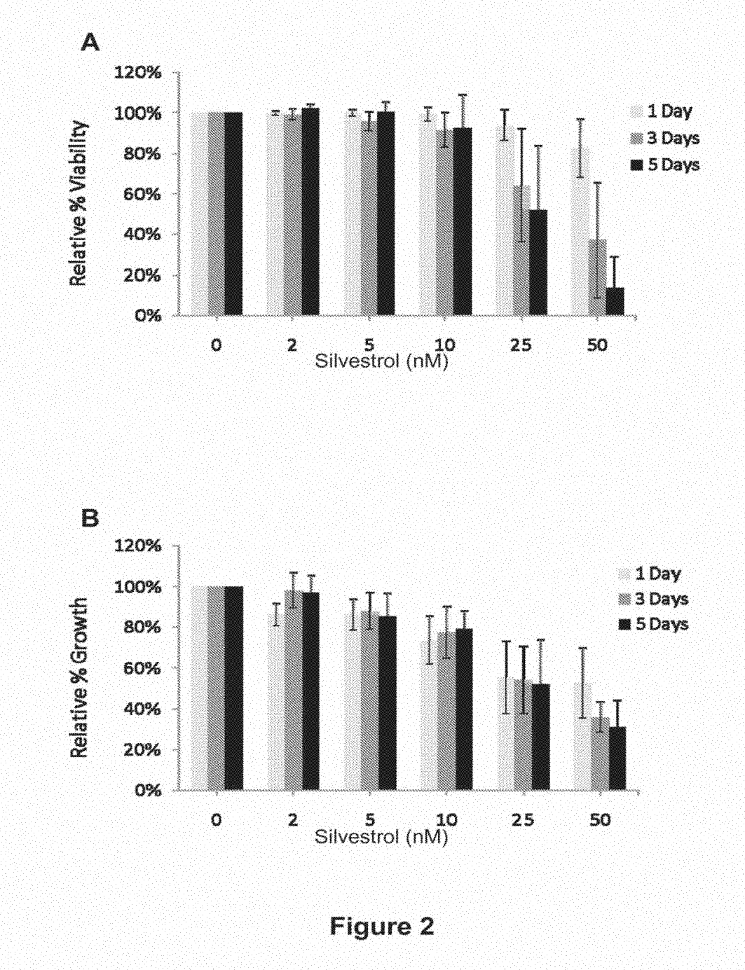

It was determined that silvestrol shows an IC₅₀ below 10 nM against most lymphoma cell lines tested. These include EBV⁺ lymphoblastoid cell lines (LCL) derived from SCID mice engrafted with peripheral blood mononuclear cells (hu-PBL-SCID) from EBV-positive donors. (FIG. 1). This hu-PBL-SCID model is a spontaneous, preclinical model of EBV-driven lymphomagenesis that can be utilized to study the anti-tumor and immune surveillance activity of EBV-specific memory T cells. In vitro, the anti-proliferative effect of silvestrol against EBV+ LCLs (FIG. 2) is preceded by a loss of EBV latent membrane protein 1 (LMP1). In FIG. 2A, relative proliferation was determined. MTS assays were performed on three donors treated with silvestrol (2, 5, or 10 nM) or DMSO (control). The assays were performed on days 1, 3, and 5. In FIG. 2B, relative growth was determined. Viability assays were performed on cells from three donors treated with silvestrol (2, 5, or 10 nM) or DMSO (control). The assays were performed on days 1, 3, and 5. Viability was assessed by annexin/propidium iodide flow cytometry.

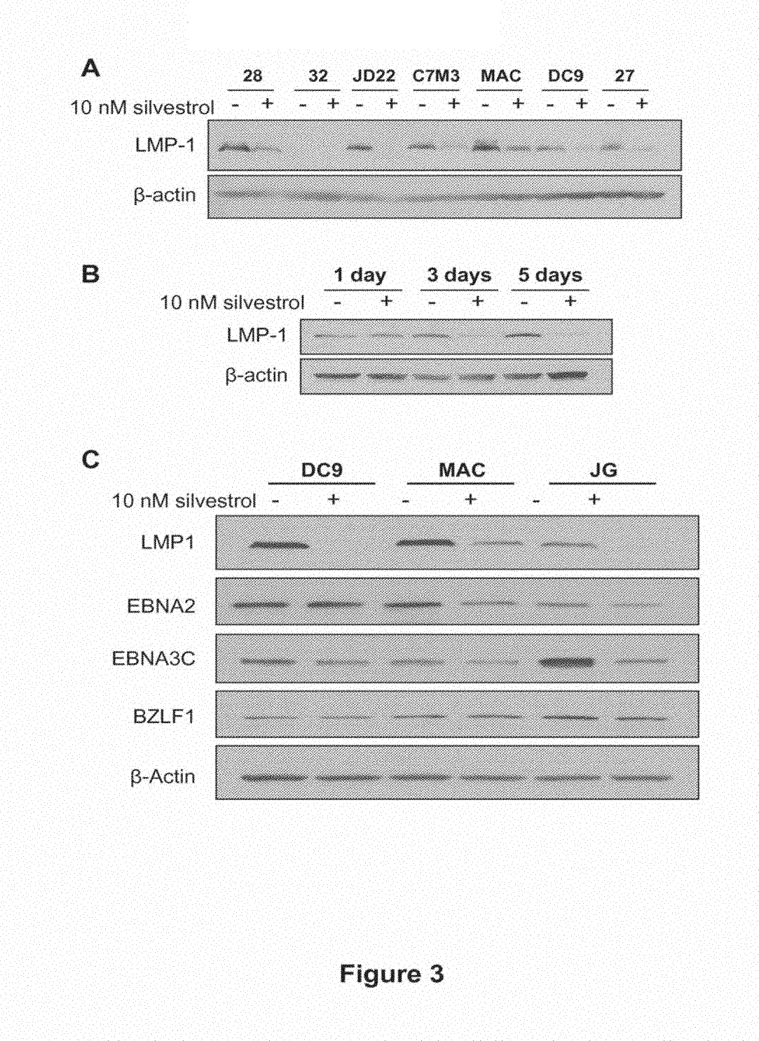

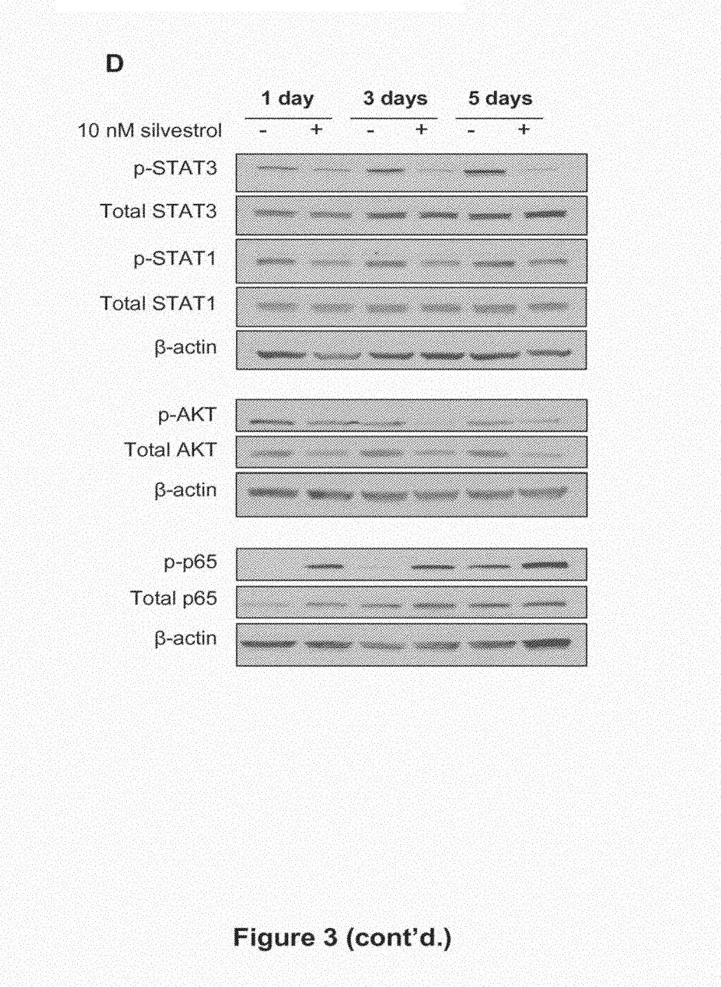

FIG. 3A shows that silvestrol treatment (10 nM) for 5 days reduced LMP1 protein levels in cells from seven different donors. β-actin was loading control. FIG. 3B shows that in cells from a single donor, silvestrol treatment reduced LMP1 levels on days 1, 3, and 5. FIG. 3C shows that silvestrol affected EBV proteins EBNA2 and EBNA3C, but did not affect the expression of BZLF1.

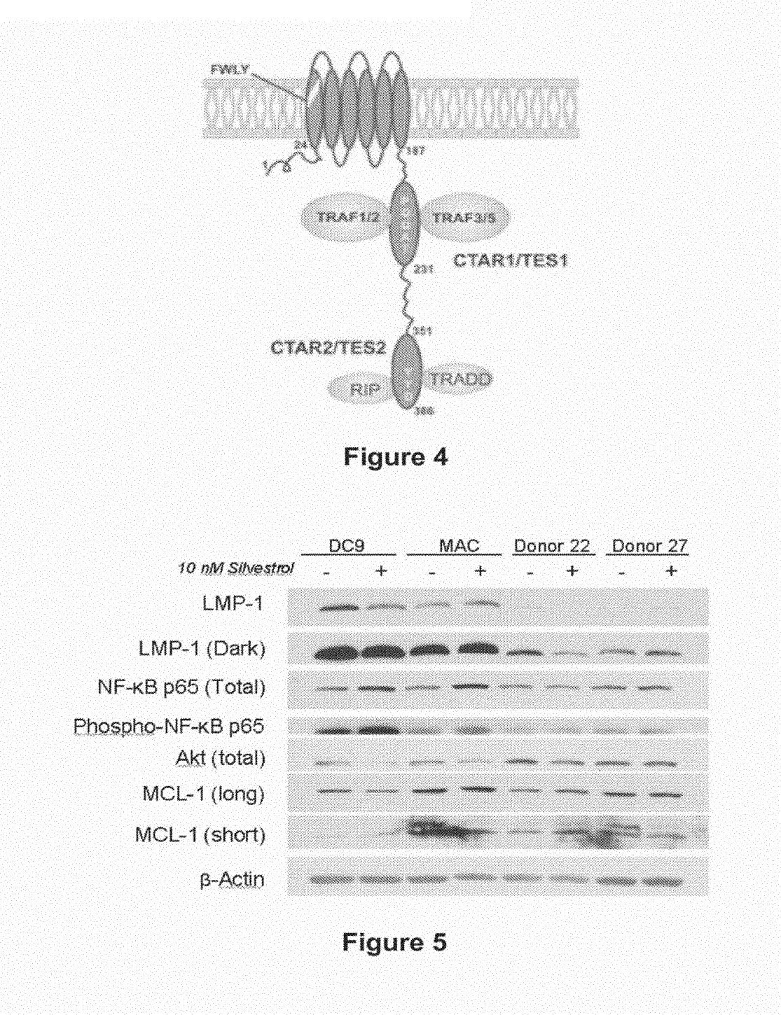

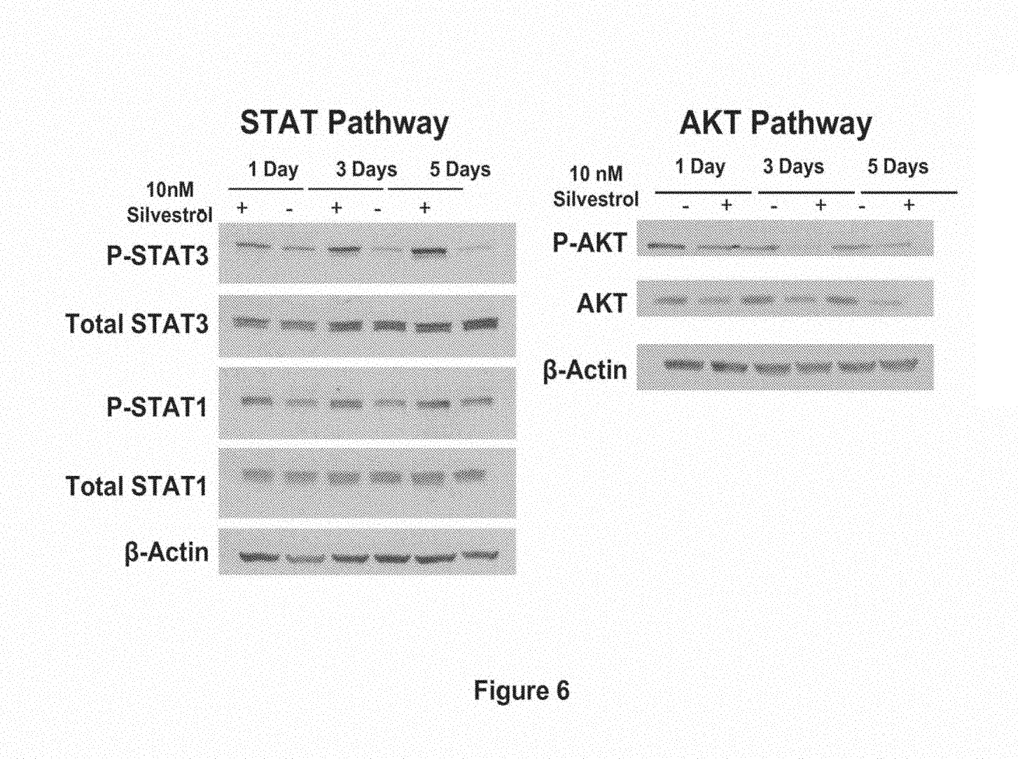

FIG. 4 shows a mechanistic schematic detailing the interaction between LMP1 CTAR domains and downstream pathways (e.g., NFκB and Akt activity). (see FIG. 3D for immunoblot data regarding STAT and Akt expression). FIG. 5 shows that silvestrol treatment reduced LMP1 and altered expression of downstream targets (see also FIG. 6), which includes the STAT and Akt pathways.

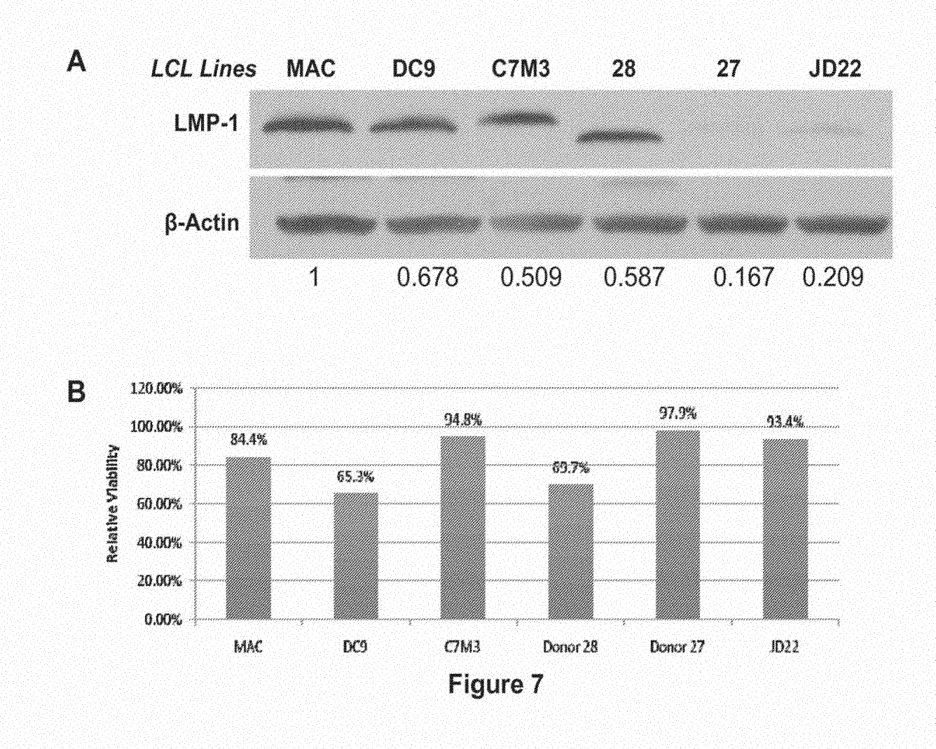

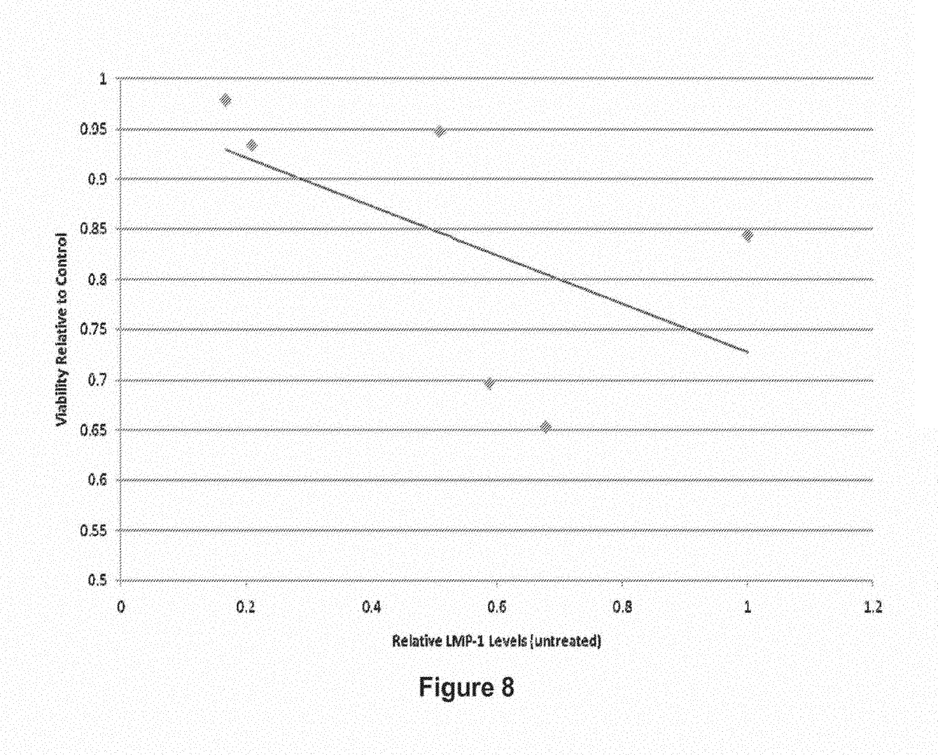

The experiments shown in FIG. 7 and FIG. 8 examined whether LMP1 expression correlated with sensitivity to silvestrol. FIG. 7A shows an immunoblot of various LCL lines for LMP-1 and β-actin (control) expression levels. FIG. 7B shows the relative viability of the LCL cells in FIG. 7A following silvestrol treatment (10 nM) for 5 days. Using LCL lines treated with silvestrol (10 nM) for five days, FIG. 8 shows viability relative to control plotted against relative LMP-1 levels (untreated), which generated an R² value of 0.2984.

Together, these data indicate that LMP1 promotes lymphomagenesis through activation of multiple survival mechanisms. The best characterized pathway is the NF-κB pathway, but additional LMP1-driven survival signals mediated by PI3-kinase, Jak/Stat, mTOR, and AP-1. Thus, LMP1 acts as an apical signal in the development of EBV-driven tumors, and thereby represents ideal therapeutic target for EBV-positive cancers.

v) Silvestrol Allows for Normal Innate and EBV-Specific Adaptive Immune Surveillance Activity at Concentrations that Provide Direct Antiproliferative Activity Against EBV+ LCLs.

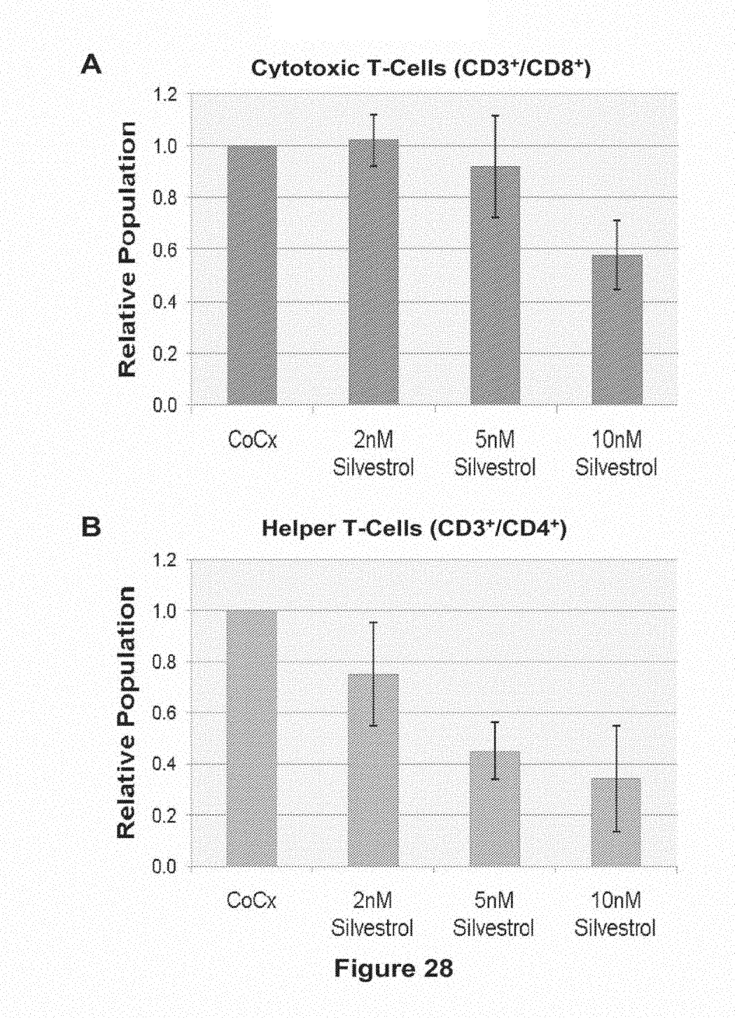

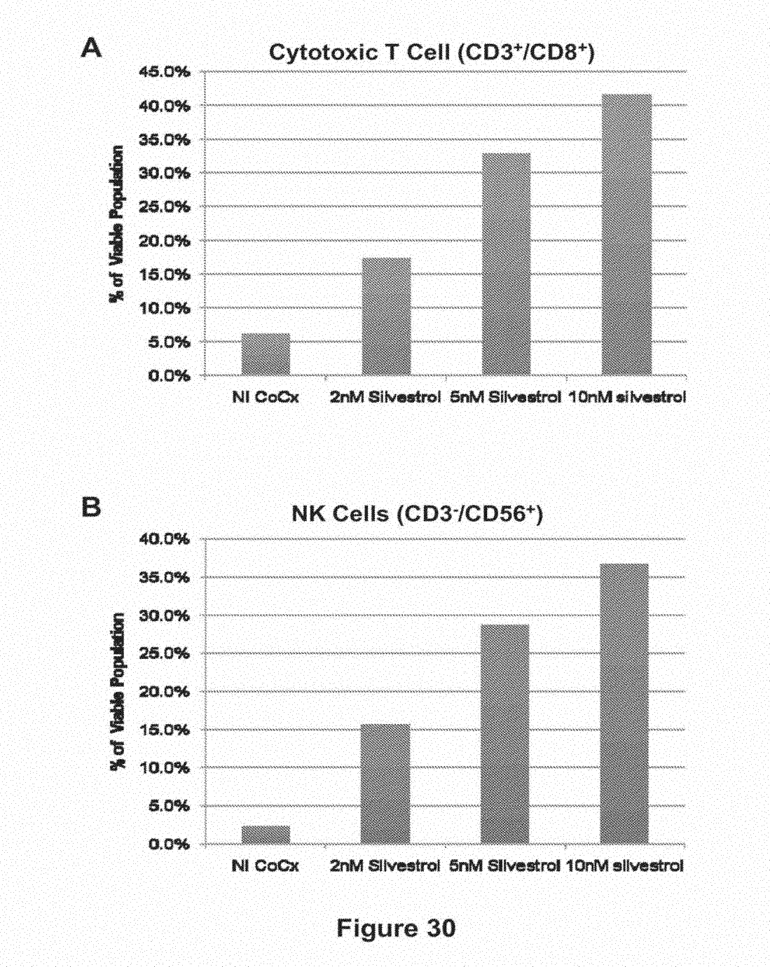

It was shown that EBV-specific adaptive T cell responsiveness was maintained at concentrations (2.5-10 nM) leading to anti-proliferative and pro-apoptotic activity of EBV⁺ LCLs. (See FIGS. 28-30). In the presence of these low silvestrol concentrations, EBV-specific CD3/CD8⁺ antigen specific T cells were capable of expansion, IFNgamma (also IFNγ) secretion, and potent antibody directed cellular cytotoxicity (ADCC) against autologous EBV⁺ LCL tumor targets.

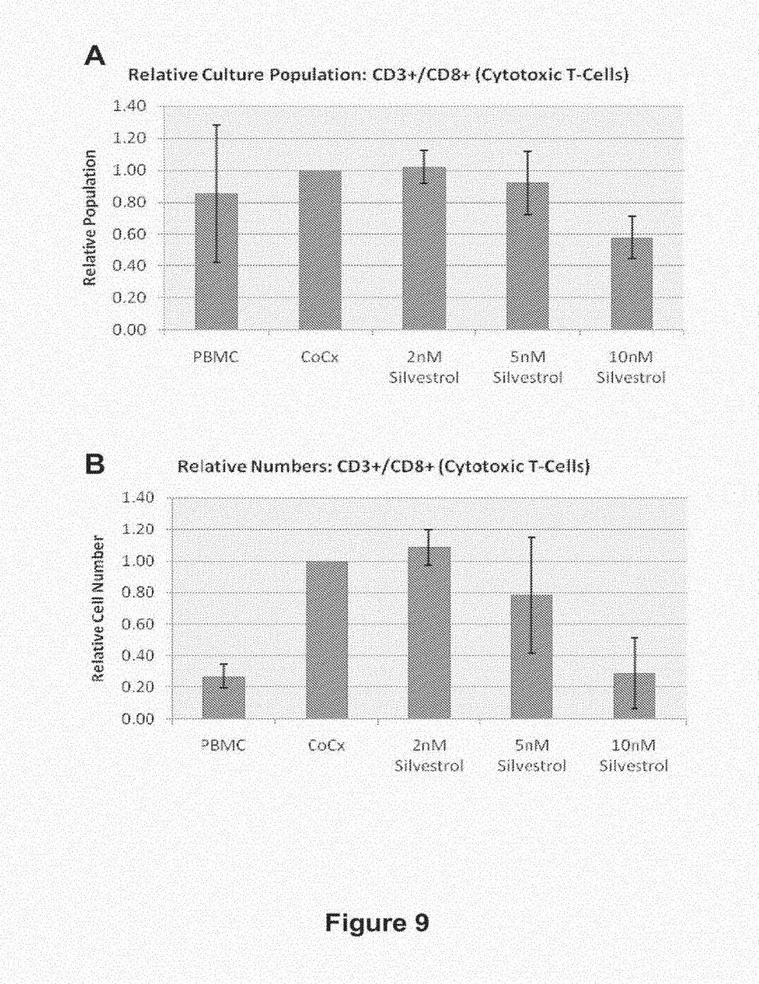

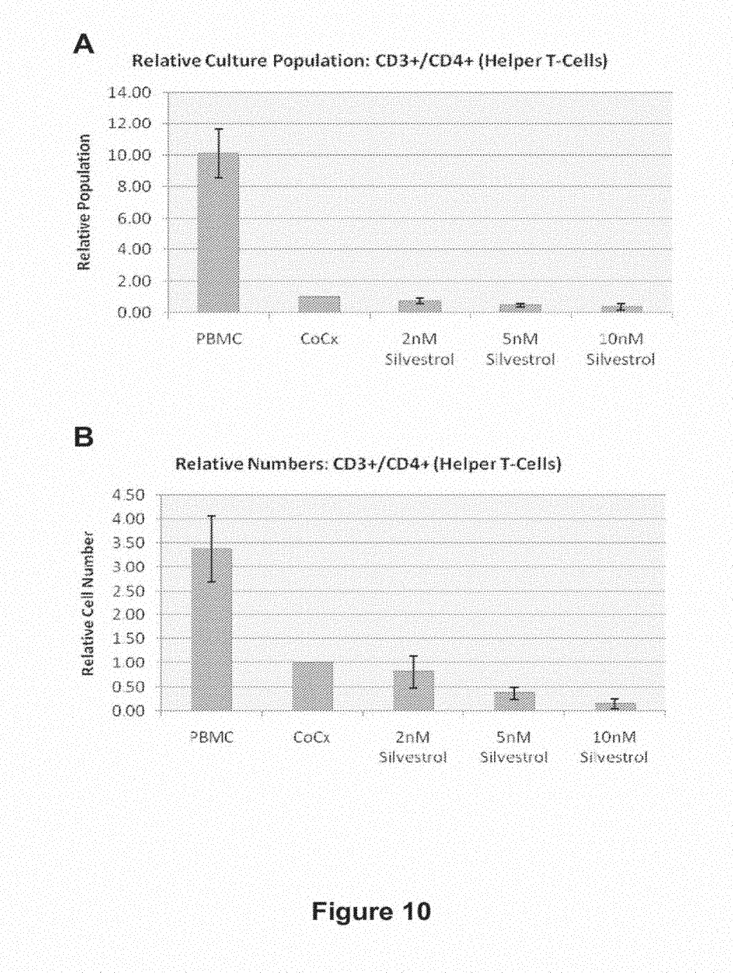

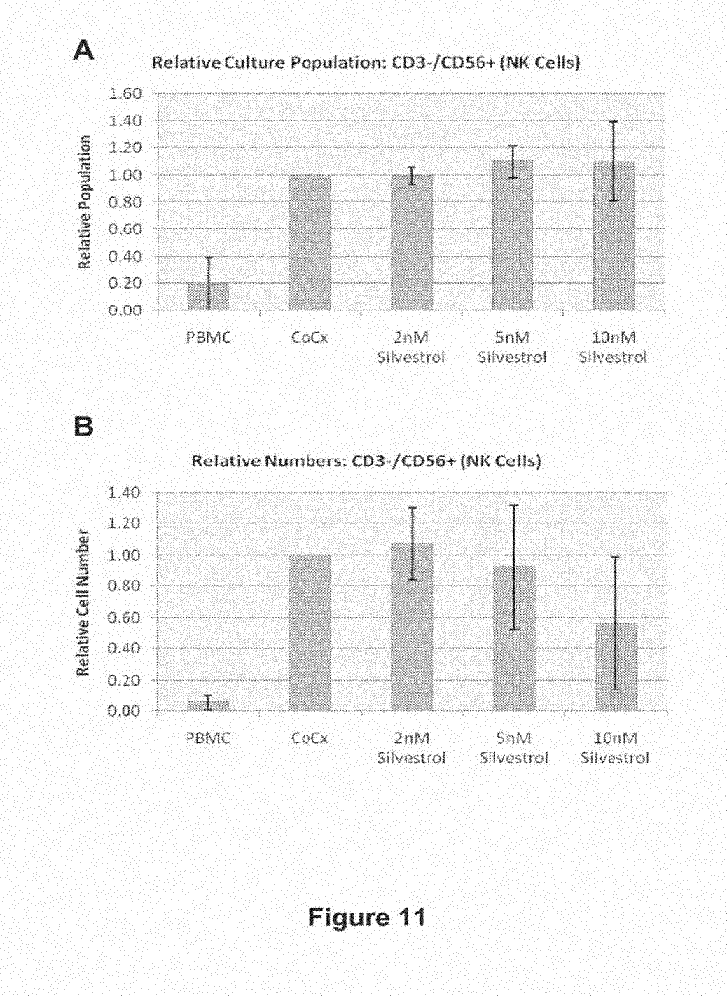

FIG. 9 shows that irradiated co-cultures of EBV-specific cytotoxic T-lymphocytes (CTL) (CD3³⁰ /CD8⁺) expanded in the presence of silvestrol at various concentrations (2, 5, and 10 nM). FIG. 9A shows the relative population of CTLs and FIG. 9B shows the relative cell number of CTLs. FIG. 10 shows data for irradiated co-cultures of helper T-cells (CD3⁺/CD4³⁰ ) under the same conditions as in FIG. 9. FIG. 10A shows the relative population of helper T-cells and FIG. 10B shows the relative cell number of helper T-cells. FIG. 11 shows data for irradiated co-cultures of NK cells (CD3³¹ /CD56⁺) under the same conditions as in FIG. 9. FIG. 11A shows the relative population of NK cells and FIG. 11B shows the relative cell number of NK cells.

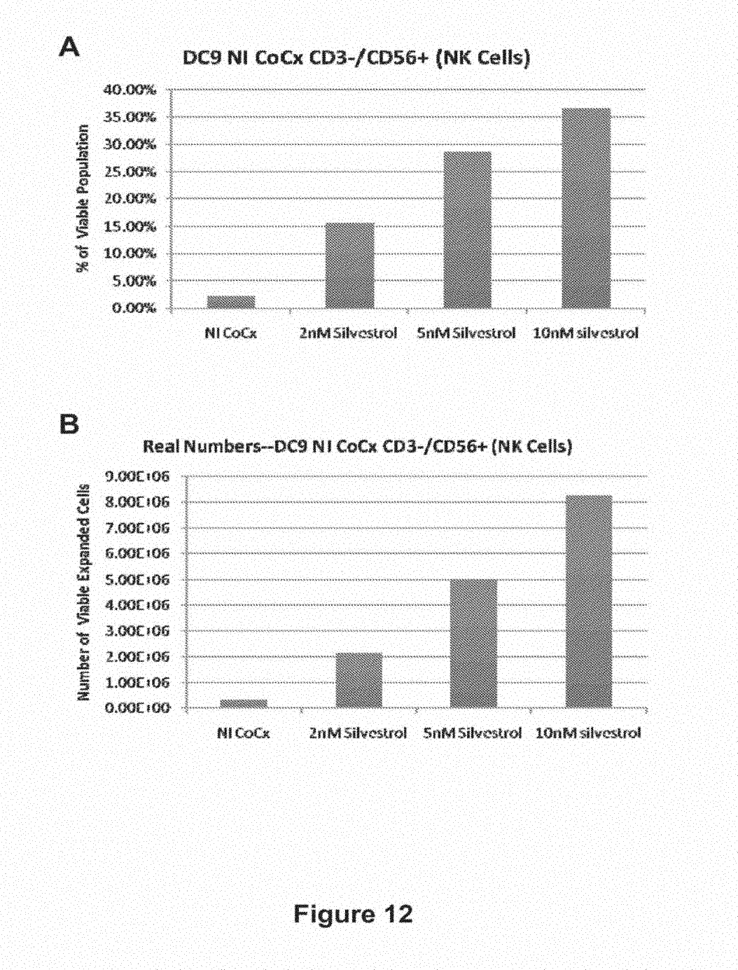

FIG. 12 shows that non-irradiated co-cultures of DC9 cells (viable tumors present along with viable autologous PBMCs) had more efficient expansion of CD3⁻/CD56³⁰ natural killer (NK) cells in the presence of silvestrol (at concentrations of 2, 5, and 10 nM) FIG. 12A shows the % of viable population of CD3⁻/CD56³⁰ NK cells at various silvestral concentrations and FIG. 12B shows the number of viable expanded cells (i.e., the real numbers) at the same silvestrol concentrations as in FIG. 12A.

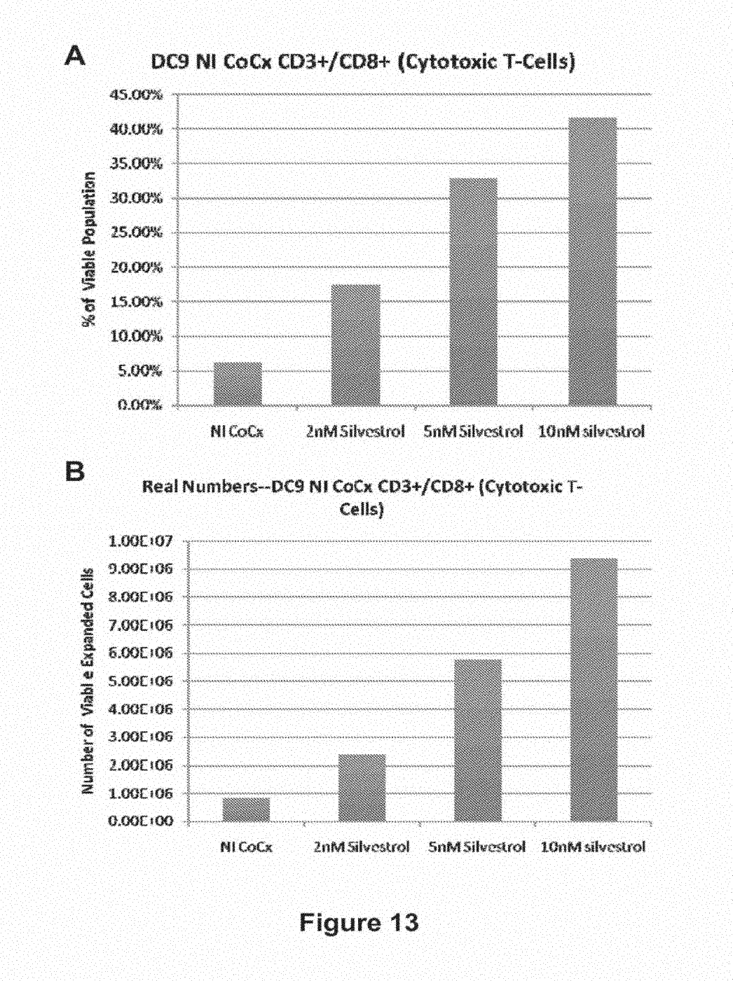

FIG. 13 shows that non-irradiated co-cultures of DC9 cells (viable tumors present along with autologous PBMCs) had more efficient expansion of CD3⁺/CD8³⁰ cytotoxic T-cells (CTLs) cells in the presence of silvestrol (at concentrations of 2, 5, and 10 nM). FIG. 13A shows the % of viable population of CD3⁺/CD8³⁰ CTLs at various silvestrol concentrations and FIG. 13B shows the number of viable expanded cells (i.e., the real numbers) at the same silvestrol concentrations as in FIG. 13A.

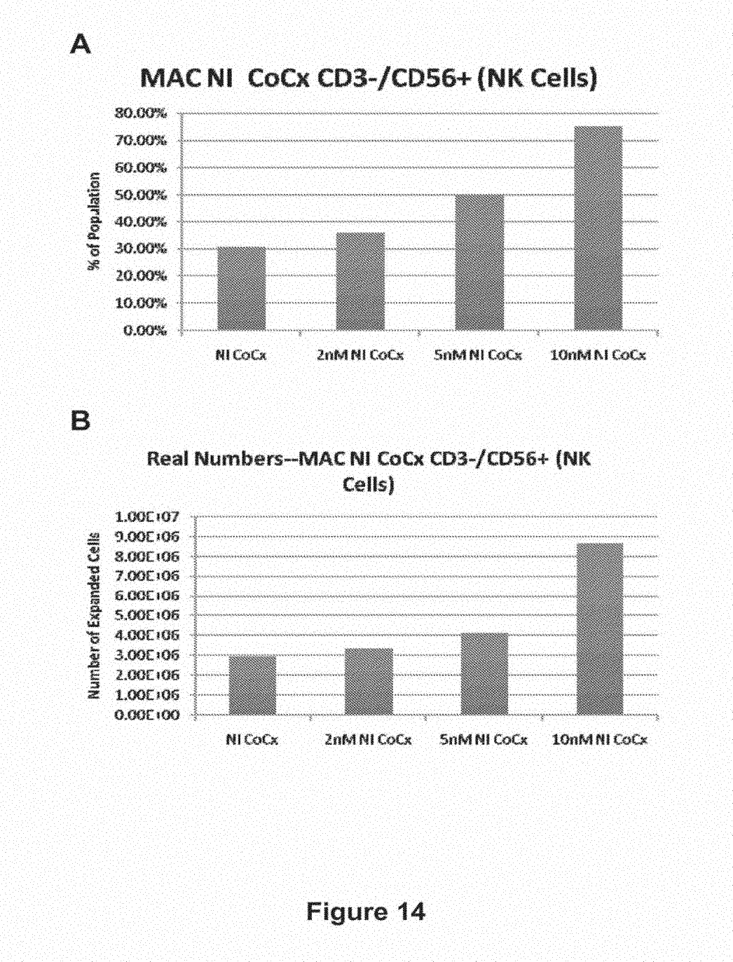

FIG. 14 shows that non-irradiated co-cultures of MAC cells (viable tumors present along with viable autologous PBMCs) had more efficient expansion of CD3−/CD56⁺ natural killer (NK) cells in the presence of silvestrol (at concentrations of 2, 5 and 10 nM). FIG. 14A shows the % of viable population of CD3−/CD56⁺ NK cells at various silvestrol concentrations and FIG. 14B shows the number of viable expanded cells (i.e., the real numbers) at the same silvestrol concentrations as in FIG. 14A.

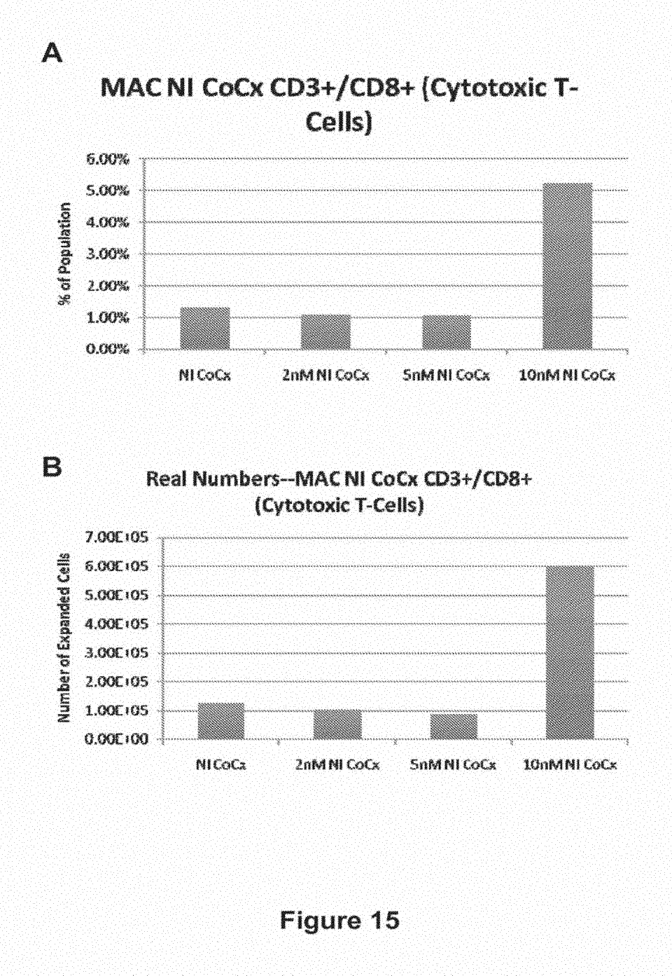

FIG. 15 shows that non-irradiated co-cultures of MAC cells (viable tumors present along with viable autologous PBMCs) had more efficient expansion of CD3⁺/CD8⁺ cytotoxic T-cells (CTLs) cells in the presence of silvestrol (at 10 nM). FIG. 15A shows the % of viable population of CD3⁺/CD8⁺ CTLs at various silvestrol concentrations and FIG. 15B shows the number of viable expanded cells (i.e., the real numbers) at the same silvestrol concentrations as in FIG. 15A.

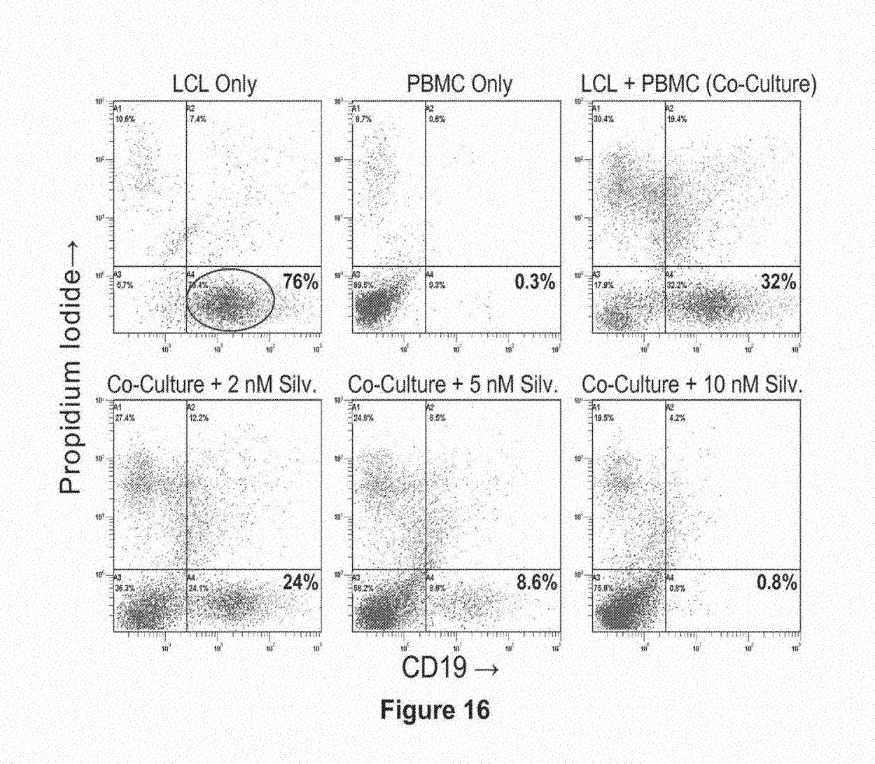

FIG. 16 shows the effect of silvestrol treatment on LCLs only, on PBMCs, and on non-irradiated co-cultures. Non-irradiated co-cultures normally showed expansion of T and NK effector cells. However, by 14 days of culture, viable EBV⁺ LCLs overgrew the culture (LCL+PBMC). In the presence of silvestrol (2, 5, and 10 nM), there was a dose dependent loss of CD19⁺ B cells and expansion of immune effector T and NK cells. Other data showed that 2-10 nM silvestrol did not lead EBV⁺ LCL tumor cells to undergo cell death, but rather, led to an antiproliferative activity.

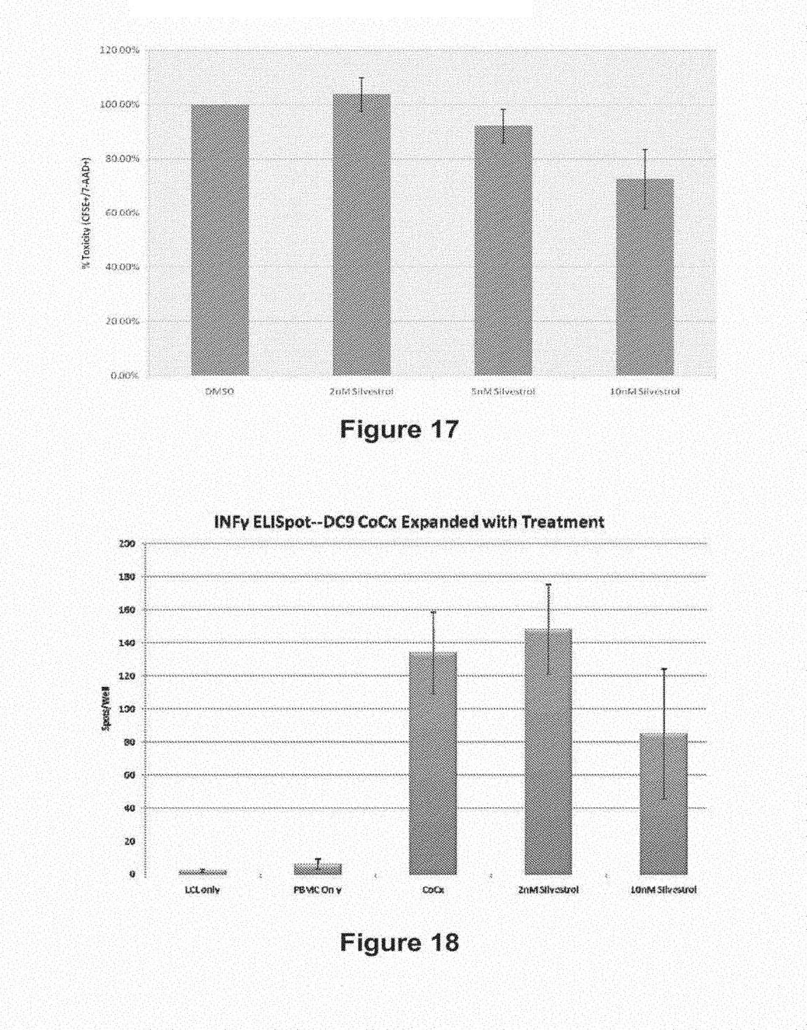

Irradiated co-cultures and non irradiated co-cultures allowed for expansion of immune effector subsets (T and NK cell) that possessed full effector function despite the presence of silvestrol (2-10nM). FIG. 17 shows cytotoxicity of immune effector subsets against EBV⁺ tumor cells (autologous) expanded for 14 days in silvestrol at various concentrations (2, 5, and 10 nM) (data represents 3 donors). The data indicate that silvestrol treatment did not affect cytotoxic activity of effector populations. FIG. 18 shows IFNgamma production of similar immune effector subsets when plated against irradiated autologous EBV⁺ LCL tumor cells (DC9 cells), indicating that IFN production is not abrogated by low concentrations of silvestrol.

vi) Silvestrol Treatment Did Not Affect ADCC Activity of NK Cells.

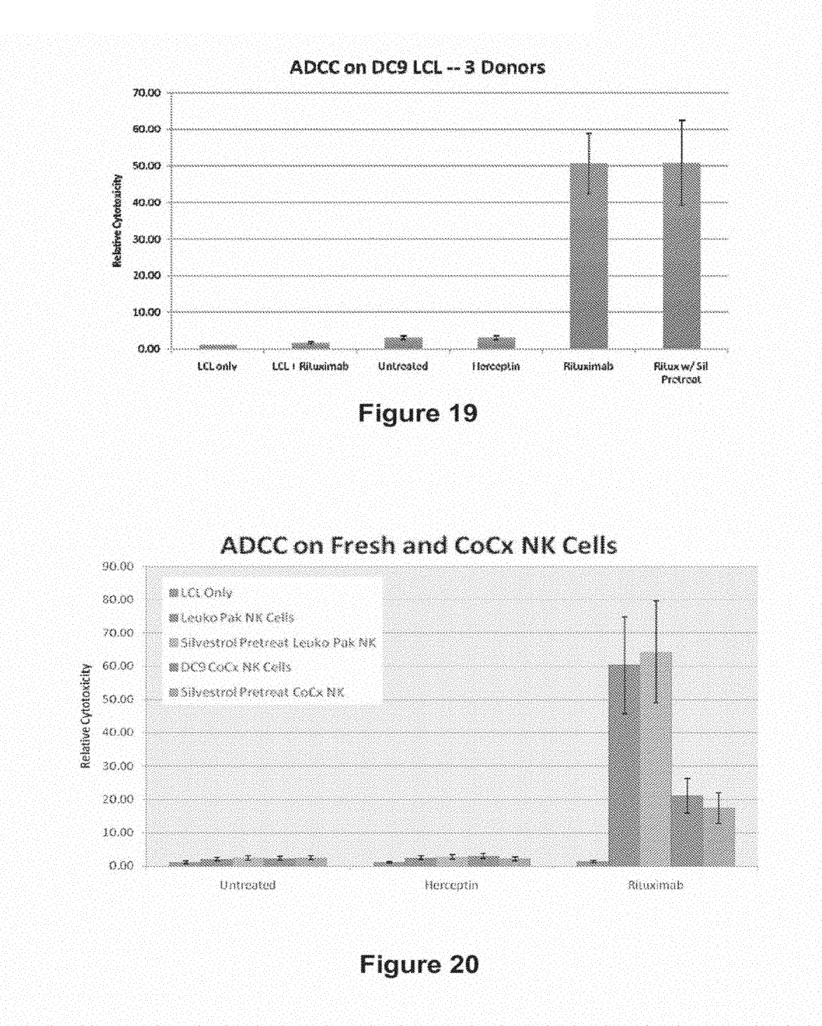

FIG. 19 shows ADCC assays on DC9 LCLs from 3 donors following various treatments. The NK cells were pretreated with silvestrol for 24 hrs prior to treatment with Rituximab or Herceptin. FIG. 19 shows equivalent ADCC activity of NK cells incubated in media vs. silvestrol (10 nM).

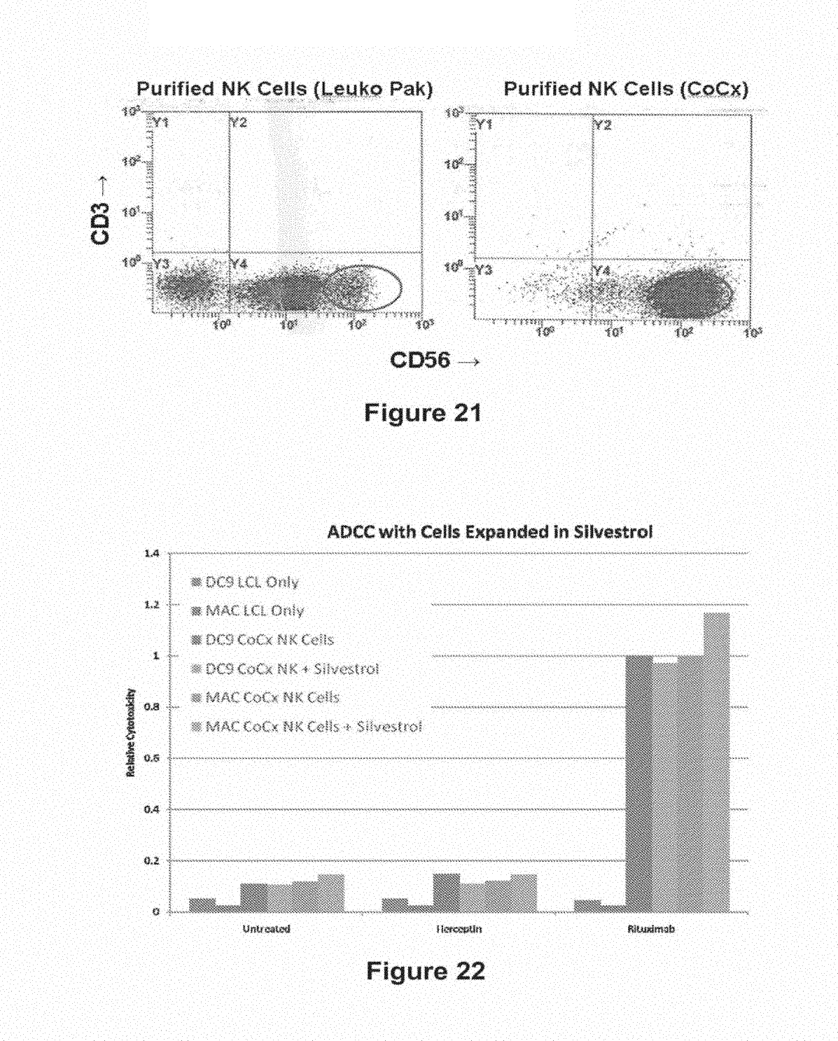

NK cells derived from 14 day cultures (irradiated and non-irradiated) also demonstrate equivalent ADCC. FIG. 20 shows ADCC with rituximab (compared to non-reactive mAb herceptin) against EBV⁺ LCL tumor cells. Luekopak NK cells were freshly isolated and exposed to either control or silvestrol (10 nM). In FIG. 20, purified NK cells were pretreated with 10 nM silvestrol for 24 hrs prior to a 4 hr flow-based cytotoxicity assay. NK cells were isolated from co-cultures that have been exposed to 10 nM silvestrol for 14 days. Silvestrol or control was also added to 4 hr cytotoxicity. Cells incubated in non-irradiated co-cultures were CD56^{bright} phenotype (IFNgamma producers, low ADCC) and cells isolated from leukopaks were CD56^{dim} (strong cytotoxicity ADCC and direct anti tumor; weak IFNgamma producers). FIG. 21 shows data regarding the expansion of NK cells that are CD56^{bright} following silvestrol treatment. FIG. 22 shows that silvestrol treatment did not affect ADCC activity of NK cells in MAC and DC9. These data indicated that (i) silvestrol allows for expansion of CD56^{bright} NK cell subsets, a critical cellular subset allowing for innate/adaptive immune networking; and (ii) that CD56^{dim} NK cells function normally when exposed to silvestrol (10 nM).

These data demonstrate that silvestrol selectively killed EBV⁺ LCL tumor cells via direct (anti proliferative) and indirect (T/NK cell immune surveillance) mechanisms. Thus, silvestrol possesses selective anti-tumor efficacy while sparing critical immune surveillance activity. Moreover, these data show the anti-tumor activity of silvestrol in EBV-driven cancers and its immune potentiating properties on innate and adaptive EBV-specific immunity.

vii) Silvestrol Efficacy in Hu-PBL-SCID EBV-LPD.

To assess silvestrol-mediated direct and indirect (immune modulation) activity, the hu-PBL-SCID model was used. Several EBV⁺ donors whose peripheral blood mononuclear cells (PBMC) generate EBV-LPD in 90-100% of mice were identified. PBMC was collected by leukapheresis, injected animals with 5×10⁷ PBMCs by intraperitoneal delivery, and assessed engraftment as measured by human Ig levels in plasma at 4 weeks.

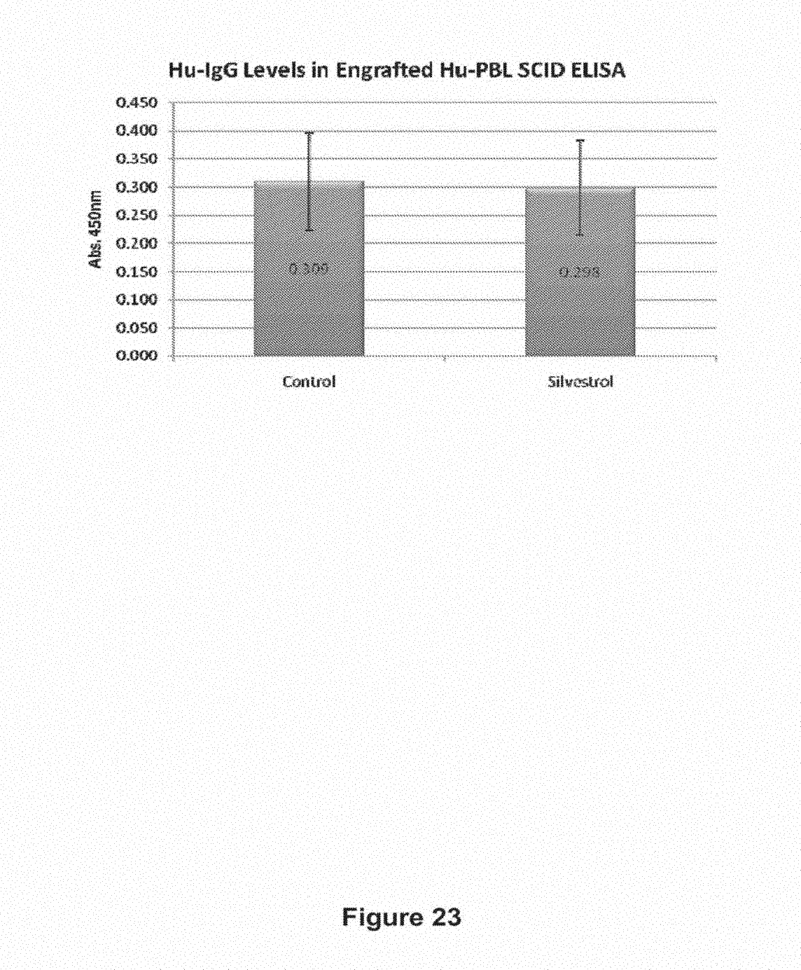

The treatment plan included treatment of experimental group with silvestrol (1.5 mg/kg every 48 hr), as this schedule demonstrated effectiveness and non-toxicity compared to control mice. Mice were randomized to receive silvestrol or vehicle control (n=16 group). Therapy started two weeks after cell injection. For immune modulation studies, mice were sacrificed and spleens harvested for flow cytometric evaluation of CD3⁺ T cell subsets and ELISPOT performed to asses functional status of T and NK cell subsets. The remaining animals (12 per group) were followed for survival. FIG. 23 shows that silvestrol therapy did not affect engraftment of human PBMCs in this model, as measured by human immunoglobulin production. Silvestrol treatment did not adversely affect humoral immune function at plasma levels in the nM range, which is relevant because vaccine responses include both adaptive T and adaptive B (humoral) cell memory and effector function. The hu-PBL-SCID model showed xenogeneic antibody titers against in murine antigens (spectrin), thus silvestrol did not affect human Ig production in this model.

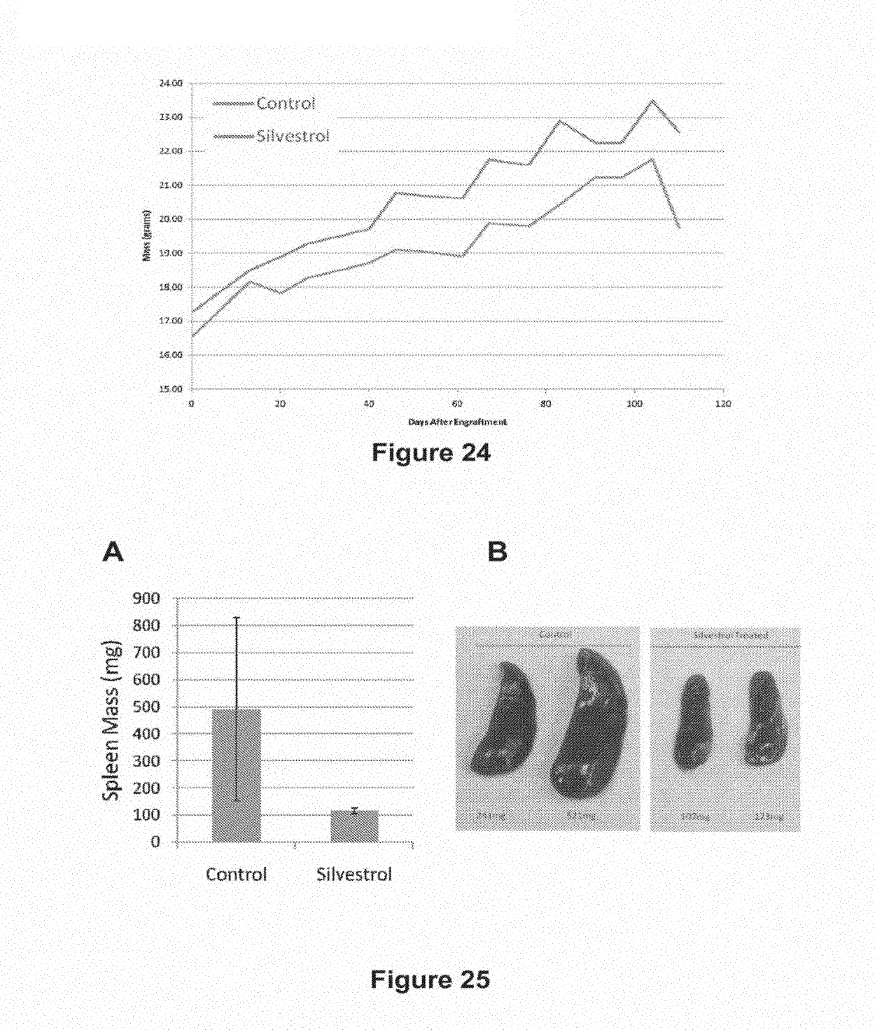

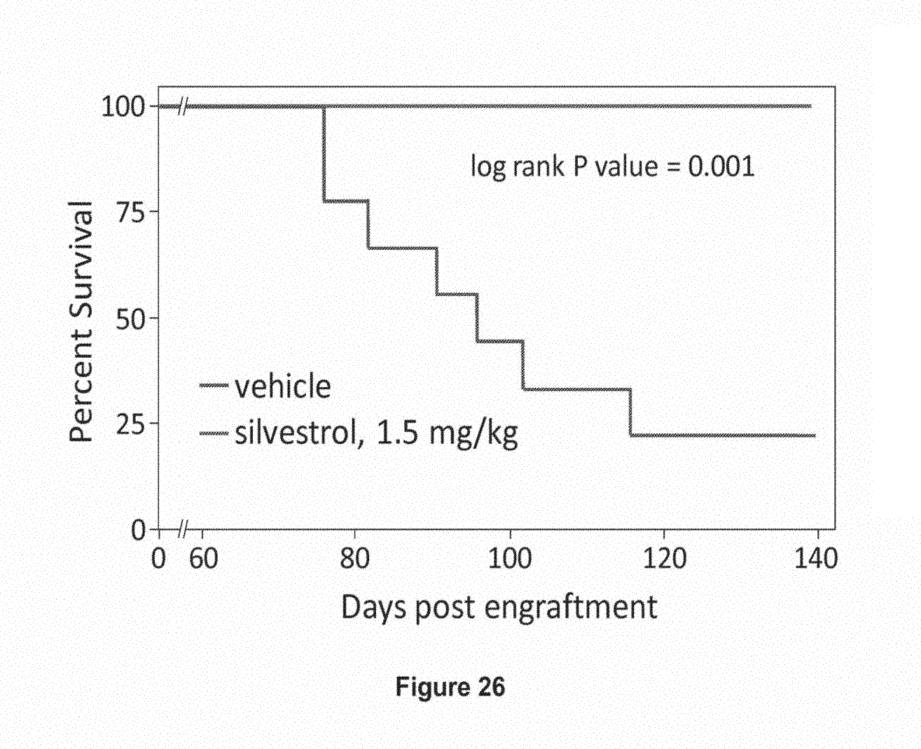

Mice receiving treatment with silvestrol showed steady improvement in total body weight (FIG. 24), smaller spleen weight (sentinel mice removed from experiment at wk 4 and 7) (FIGS. 25A and 25B), and overall survival (FIG. 26).

viii) Summary of Findings:

Silvestrol has shown direct anti-tumor activity (both decrease in proliferation as well as loss of viability). Cytotoxic effects of silvestrol on immune effector subsets (CD3/CD8 CTL, NK CD56^{bright}, and NK CD56^{dim}) is minimal to absent. Immune effectors still potentiated a robust response after an acute or long-term treatment (both adaptive and innate). IFNgamma production remained normal, humoral function/antibody production is unaffected by silvestrol, and direct/ADCC cytotoxicity of NK cell and CTL subsets was unaffected by silvestrol. Oncogenic EBV proteins such as LMP-1 are down-regulated as a result of Silvestrol treatment. Mouse studies showed no toxicity and statistically significant survival.

ix) Applications of Silvestrol:

Because silvestrol allows for the expansion of immune effectors while inhibiting tumor growth, allowing for preservation of anti tumor activity, it is clinically relevant for a number of applications. The first application involves synthesis of novel drug compositions allowing for prevention and treatment of disease. These include vaccine compositions comprised of silvestrol (as the immune adjuvant component) and (1) vaccine (viral); (2) vaccine (bacterial); and (3) vaccine (tumor). Presently, there are no effective methods or compositions that have demonstrated efficacy as a therapeutic vaccine. The EBV data shows that EBV/tumor specific activity is maintained while tumor growth is suppressed, thus leading to direct and indirect-immune-mediated anti tumor activity. Thus, silvestrol can be included as an immune adjuvant in any vaccine with the intent to prevent or treat existing disease.

Other applications include silvestrol as part of a composition to promote immune surveillance in vitro and/or in vivo. These include combinations with: cytokines; cellular therapeutics (innate/NK); and cellular therapeutics (adaptive/T cell/CTL).

### B. Example 2

### EBV-Driven Lymphoma Model: Hu-PBL-SCID Mice

Epstein-Barr virus (EBV) is an oncogenic, lymphotropic herpesvirus. It is implicated in a wide range of B-cell lymphoproliferative disorders, such as Hodgkin's lymphoma, Burkitt's lymphoma, nasopharyngeal carcinoma, Diffuse Large B Cell Lymphoma in immunocompromised hosts, e.g., AIDS patients, post-transplant patients. Chemo/immunotherapy for such patients provides sustained response only in small percentage, and leads to immune suppression, increased risk of relapse, and opportunistic infections. Efficacy of silvestrol against B-leukemia cells, plus relatively low activity against normal peripheral blood mononuclear cells (PBMC), can provide anti-tumor activity in EBV lymphoma while maintaining normal immune function.

Six week-old female SCID mice (n=28) received anti-asialo antisera prior to engraftment to deplete murine NK cells. Mice were engrafted intraperitoneally with 5×10⁷ peripheral blood mononuclear cells (PBMCs) obtained from EBV-positive, healthy donor. Untreated mice in this model develop lethal disseminated (human) lymphoma; median survival varies by PBMC donor for that experiment. Two weeks post-engraftment, treatments were initiated with either vehicle (30% HBPCD) or vehicle+silvestrol (1.5 mg/kg every 48 hrs; n=14 each). 4 and 8 weeks post-engraftment, 2 mice from each group were sacrificed for examination and blood was drawn from all mice to assess human IgG levels (FIG. 23). The remainder of mice (n=10 each) were followed for survival. Survival is defined as absence of IACUC-approved euthanasia criteria (in this model, typically this is weight loss >20%, together with labored breathing and cessation of grooming).

### C. Example 3

### Silvestrol Modulates Direct Anti-Tumor Activity Against Epstein-Barr Virus (EBV)-Associated Lymphomas While Sparing Innate and Antigen Specific Adaptive Immunity

Epstein-Barr virus (EBV) is an oncogenic human herpes virus that infects more than 90% of people worldwide and is associated with a broad spectrum of malignant lymphoproliferative disorders (EBV-LPD), and nasopharyngeal and gastric carcinomas. Chemotherapy often leads to prolonged immune suppression, development of opportunistic infections, including EBV reactivation, and increased risk of relapsed disease. The poor prognosis of EBV+ diseases makes it essential to identify novel agents that can deliver direct anti-tumor activity while preserving innate and EBV-specific adaptive immune surveillance.

Silvestrol is a cyclopenta[b]benzofuran derived from the plant genus Aglaia and has been shown to possess potent anti-tumor activity against hematologic and solid tumors. Silvestrol interferes with the translation of mRNA with complex 5′ untranslated regions often found on pro-survival oncoproteins. Silvestrol exhibits anti-tumor activity against malignant B-cell lines while causing minimal toxicity to peripheral blood mononuclear cells (PBMC) and resting T cells.

To examine the potential selective anti-tumor activity and functionally address the effects of silvestrol on adaptive and innate immune function, in vitro and in vivo EBV lymphomagenesis models were utilized. Fully transformed EBV+ Lymphoblastoid lines (LCL) were derived from EBV-LPD tumors of severe combined immune deficient (SCID) mice engrafted with PBMC from EBV-positive donors (hu-PBL-SCID). (FIG. 1). EBV-LCLs were plated in the presence of silvestrol (2-50 nM) and the level of proliferation (MTS assay) and apoptosis (Annexin V/PI) were evaluated (24, 72, and 120 hr) (FIGS. 2A and 2B). The anti-proliferative activity of silvestrol was associated with loss of LMP-1 expression, an EBV oncogene essential for B-cell transformation (FIG. 4). Examination of LMP-1 induced pathways showed decrease in pAkt levels (FIG. 6) and an increase in NFkB/p65 levels (total and phospho) (FIG. 6).

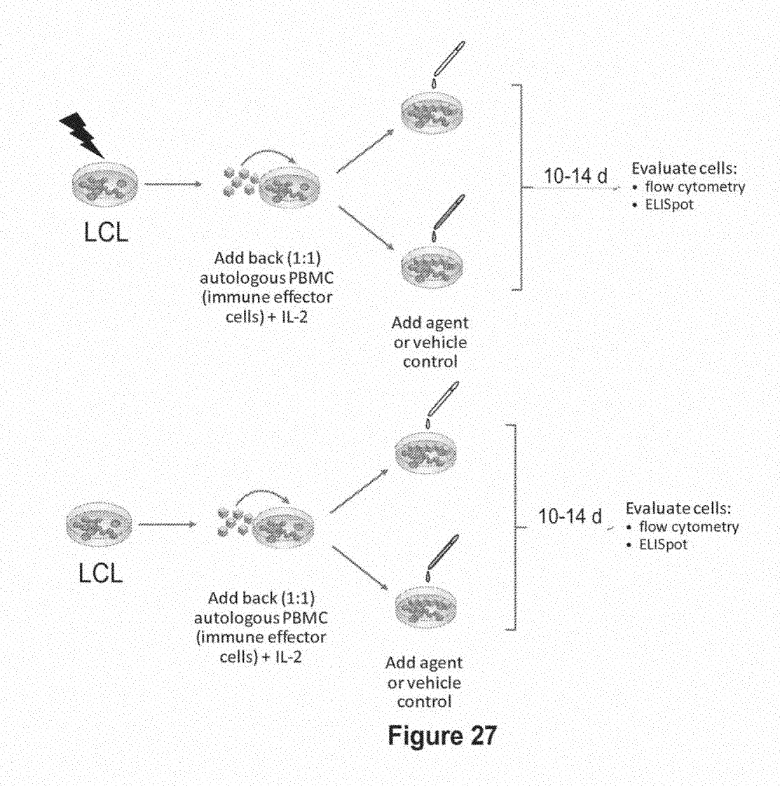

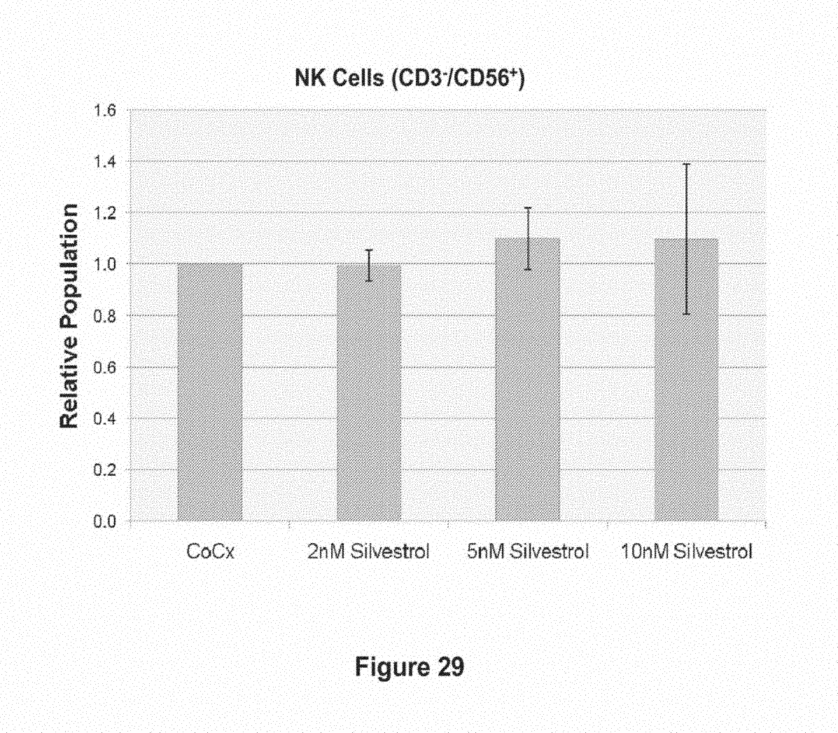

To examine the functional consequences of silvestrol on immune surveillance, a co-culture system was utilized where EBV+ LCLs are plated in the presence of autologous PBMCs (FIG. 1). Autologous LCL/PBMC co-cultures were plated with silvestrol or DMSO vehicle control and allowed to incubate for 10 days (FIG. 27). When EBV⁺ LCLs were irradiated prior to the addition of autologous PBMC, expansion of memory EBV-specific CD3+/CD8 cytotoxic T cells (CTLs), capable of cytotoxicity and IFNgamma production, was observed. The addition of silvestrol (2-10 nM) to co-cultures did not hinder CTL expansion or IFNgamma production. When non-irradiated LCLs were plated in co-cultures, CTL expansion was still seen. However, EBV+ LCLs became the dominant population in control conditions by day 10 of culture. Addition of silvestrol to unirradiated co-cultures (2-10 nM) led to marked expansion of memory CD3/CD8+ CTLs as well as CD56^{bright} NK cells. (See FIGS. 29-32). A dose dependent ablation of viable EBV-LCLs was observed in unirradiated co-cultures supporting the notion that silvestrol allowed for the expansion of both innate and adaptive immune effectors that were capable promoting anti-tumor activity. Immune effector populations that expanded in the presence of silvestrol showed preservation of direct cytotoxicity (adaptive immunity) and antibody dependent cell-mediated cytotoxicity assays (ADCC) against EBV+ LCLs (innate immunity) that was comparable to untreated effector populations.

### D. Example 4

### Silvestrol Modulates Direct and Indirect Anti-Tumor Activity Against Epstein-Barr Virus-Driven Lymphoproliferative Disease

Treatment options for patients with Epstein-Barr Virus-driven lymphoproliferative diseases (EBV-LPD) are limited. Chemotherapy, immunotherapy, and stem cell transplantation often lead to profound immune suppression, increasing the risk of lethal opportunistic infections and EBV reactivation. The poor prognosis associated with EBV-LPD makes it essential to identify novel agents that can deliver direct anti-tumor activity while preserving host innate and EBV-specific adaptive immune surveillance. Silvestrol is a novel translation inhibitor that has been shown to possess potent anti-tumor activity against multiple hematologic malignancies while causing minimal toxicity to normal peripheral blood mononuclear cells and resting T cells. Here, silvestrol has direct anti-tumor activity against EBV-transformed lymphoblastoid cell lines (LCL), exhibited by growth inhibition, decreased expression of the oncogenic EBV latent membrane protein-1, and down-modulation of AKT, STAT1 and STAT3 pathways. Silvestrol promotes indirect anti-tumor effects by preserving expansion of innate and EBV antigen-specific adaptive immune effector subsets that remain capable of delivering direct and antibody-mediated cytotoxic activity to LCL tumor targets. In a human-murine chimeric xenograft model of spontaneous EBV-LPD, silvestrol demonstrates therapeutic activity without observable toxicity.

i) Introduction

Epstein-Barr virus (EBV) is a human lymphotropic gamma herpes virus that infects more than 90% of people worldwide (Kieff2001). The virus is highly oncogenic, and can transform human B lymphocytes and epithelial cells in vitro and in vivo. EBV is associated with a spectrum of malignant diseases including Burkitt's lymphoma, non-Hodgkin's and Hodgkin's lymphomas, nasopharyngeal and gastric carcinomas, and post-transplant lymphoproliferative disease (LPD). Following primary infection, the virus establishes persistent, life-long latency in the B-cell compartment of the human host. This virus/host coexistence is controlled by a highly efficient antigen-specific adaptive immune response that protects immune competent individuals from EBV-driven pathology. EBV-positive individuals who become immunocompromised are at risk for EBV reactivation and development of aggressive lymphomas. Current treatments for patients with EBV-driven lymphomas are of limited benefit, and also lead to further immune suppression, opportunistic infections and a loss of EBV-specific immunity due to dysregulation of immune surveillance (Park 2007). Therefore, novel treatment approaches that maintain host immune function are needed.

Silvestrol is a unique agent isolated from the Indonesian plant Aglaia foveolata (Hwang 2005) that has been shown to possess direct anti-tumor activity in multiple cancer types (Kim 2007; Mi 2004). The anti-cancer property of silvestrol is attributed to inhibition of translation initiation that occurs when silvestrol induces the aberrant dimerization of the RNA helicase eIF4A with capped mRNA (Bordeleau 2007; Cencic 2009). This effect can interfere with normal recruitment of mRNA to the eIF4F initiation complex, thus preventing the rapid synthesis of pro-survival and pro-growth proteins and leading to tumor cell death via caspase-dependent apoptosis. It was shown that silvestrol shows in vitro and in vivo activity in the B-cell malignancies chronic lymphocytic leukemia and acute lymphoblastic lymphoma, and also that silvestrol is selectively cytotoxic to malignant B cells while sparing normal lymphocytes.

Here, silvestrol promoted direct anti-tumor activity against EBV-driven lymphoma by blocking oncogenic pathways driven by the EBV gene product, latent membrane protein 1 (LMP-1), LMP-1 is essential for B cell transformation by acting as a constitutively active tumor necrosis factor receptor (TNFR) and has been shown to be a promising therapeutic target in EBV malignancies (Lucas 2009). Furthermore, it was demonstrated that in addition to its direct activity against tumor cells, silvestrol preserved the anti-tumor function of adaptive and innate immune effector populations, conferring at strong anti-tumor effect in both in vitro and in vivo models of EBV-driven lymphoma. This unusual selectivity showed that silvestrol can provide an entirely new therapeutic strategy for this histologic subset of aggressive lymphomas.

ii) Materials and Methods

(I) Reagents. Silvestrol was Isolated as Described (Hwang 2004).

(a) Cells and Cultures.

Lymphoblastoid cell lines (LCL) were derived in vivo by engrafting severe combined immune-deficient (SCID) mice (Taconic, Hudson N.Y.) with human peripheral blood mononuclear cells (PBMC) from a healthy EBV-positive donor as described (Rowe 1991; Baiocchi 1994). Co-cultures (Cocx) were created by mixing 5+10⁴ LCL (either non-irradiated or irradiated with 14000 rad) per well with equal numbers of autologous PBMC in 96-well plates. Cultures were grown in the presence of 10 U/mL IL-2 and were given a single dose of silvestrol and cultured for 10-14 days.

iii) Immunoblot Analysis.

Cells were harvested and lysed as described (Alinari 2011). Following collection of whole cell lysates, equal amounts of protein were resolved by SDS-PAGE and transferred onto a PVDF membrane (Millipore, Billerica, Mass.). Membrane was probed overnight at 4° C. with β-actin, pSTAT1, pSTAT3, STAT1, STAT3, pAkt, Akt (Cell Signaling Technology, Danvers, Mass.), or LMP-1 (DakoCytomation, Carpinteria, Calif.). After incubation with HRP-conjugated secondary antibody (Cell Signaling Technology), protein was detected with SuperSignal West Pico Chemiluminescent Substrate (Pierce, Rockford, Ill.).

iv) Proliferation Assays.

MTS proliferation assays were performed according the CellTiter 96™ Aqueous Cell Proliferation Assay Technical Bulletin (Promega, Madison, Wis.). 5×10⁴ cells were plated in each well of a 96-well plate at 37° C. and assayed for proliferation at 24, 72, and 120 hr.

Analysis by Flow Cytometry.

Cells from irradiated or non-irradiated co-cultures were co-stained with LIVE/DEAD cell stain (Invitrogen, Grand Island, N.Y.) and hu-CD3-APC and either hu-CD4-PE, hu-CD-8-PE, hu-CD19-PE (BD Biosciences, San Diego, Calif.), or hu-CD56-PE (Beckman Coulter, Bren, Calif.). Live events were gathered by gating on cells negative for the LIVE/DEAD stain on an FC500 cytometer (Beckman Coulter). Cell viability was also measured by flow cytometry at 24, 72, and 120 hr using annexin-V-FITC and propidium iodide (PI) according to the manufacturer's instructions (BD Biosciences).

vi) Cytotoxicity Assays.

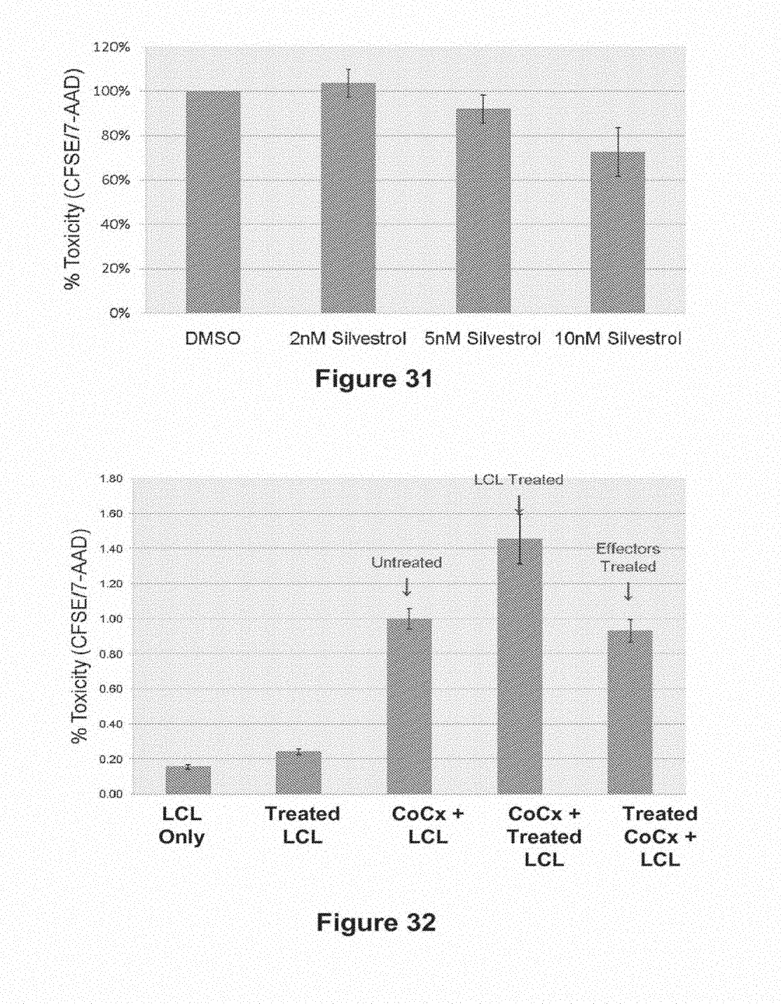

Non-radioactive flow cytometry-based cytotoxicity assays have been described previously (Godoy-Ramirez 2005; Helguera 2011). Co-cultures of PBMC and irradiated LCL were grown in the presence or absence of silvestrol for 14 d and collected. Fresh autologous LCL cells were stained with 200 nM carboxyfluorescein diacetate succinimidyl ester (CFSE) (CellTrace™, Invitrogen) for 15 min at 25° C. prior to being mixed with effectors from the irradiated co-cultures at an effector:target (E:T) ratio of 20:1 for 4 hr at 37° C. After the incubation, cells were stained with 7-aminoactinomycin D (7AAD) (BD Biosciences, San Diego, Calif.) and washed. Cytotoxicity was measured by gating on the CFSE-positive events and measuring the 7AAD -positive cells. (See FIGS. 31 and 32). For antibody-dependent cell-mediated cytotoxicity (ADCC) assays the same technique was used; however, the co-cultured effectors were natural killer (NK) cell-enriched and were incubated in the presence or absence of 5 μg/mL rituximab herceptin (negative control) (Genentech, Inc., South San Francisco, Calif.) for 4 hr at 37° C.

vii) In Vivo Studies.

The Hu-PBL-SCID mouse model has been described (Mosier 1992). Human PBMCs were obtained from a healthy EBV seropositive donor. 5×10⁷ cells were injected intraperitoneally (IP) into 6 week-old female SCID mice (Taconic) that were NK cell-depleted by pretreatment with anti-asialo (GM1) (Wako Pure Chemical Industries, Richmond, Va.). Engraftment was confirmed by hu-IgG ELISA from serum as described (Baiocchi 1994). Silvestrol treatment began two weeks post-engraftment.

b) Results

i) Silvestrol Promotes Direct Anti-tumor Activity Against EBV-Positive Lymphoblastoid Cell Lines.

The direct anti-tumor activity of silvestrol in leukemia and lymphoma models has been document (Hwang 2004; Bordeleau 2008; Cencic 2009; Lucas 2009 Alinari 2011; Saradhi 2011) as well as silvestrol's increased cytotoxicity to malignant B cells compared to normal blood lymphocytes (Lucas 2009). However, activity of silvestrol has not previously been described in spontaneous EBV-transformed cell lines. Silvestrol's activity in fully transformed EBV-positive LCL derived from tumors of SCID mice engrafted with PBMC from EBV-seropositive donors was observed. Six of these EBV-LCL lines were plated in the presence of low concentrations of silvestrol (2-50 nM), and apoptosis (viability assessed by annexin/PI flow cytometry) (FIG. 2A) and proliferation (MTS assay) (FIG. 2B) were evaluated at 24, 72, and 120 hr. Cells were plated at a sufficiently low density so that even after five days there was minimal loss of viability due to nutrient depletion in the untreated control. In FIG. 2A, data are shown as percent annexin-negative and PI-negative cells relative to the time-matched vehicle control. Bars show −/+ standard deviation. In FIG. 2B data are shown relative to the vehicle control at each time point, and bars show −/+ standard deviation. Moderate but significant activity was noted both in growth inhibition and apoptosis (p<0.0001 and p=0.006 respectively vs. untreated control, averaged over doses and time points), with an estimated 50% growth inhibitory concentration (IC₅₀) of approximately 40 nM at 72 hr. Pharmacokinetics work in mice indicated that a 10 nM dose of silvestrol is attainable in vivo. Thus, for mechanistic and functional immunological assays, 10 nM and lower doses that were found to be minimally toxic in these direct anti-tumor assays were used.

ii) Silvestrol Induces LMP-1 Depletion in LCL Lines.

The effect of silvestrol on viability and proliferation of LCL indicated an interfere with EBV transforming proteins. The virally-encoded transmembrane protein LMP-1 acts as a constitutively active receptor mimicking CD40, a member of the tumor necrosis factor receptor family (Gires 1997). LMP-1 is expressed in multiple EBV-associated malignancies including Hodgkin's lymphoma, nasopharyngeal carcinoma, and diffuse large B cell lymphoma. This viral gene product promotes multiple growth pathways as well as suppresses immune-activating cytokines, and is essential for B cell transformation (Najjar 2005; Shair 2007). It has also been shown that because of these properties, LMP-1 is an effective therapeutic target in EBV-driven malignancies (Kenney 2001; Mei 2007; Hannigan 2010; Yang 2010).

Whole cell protein levels of LMP-1 was evaluated by immunoblot 72 hr after treating with a single dose (10 nM) of silvestrol in seven LCL lines (FIG. 3A). β-actin was included as a loading control. In FIG. 3B, LCL were incubated 24, 72, and 120 hr in the presence of 10 nM silvestrol or vehicle control. Whole cell lysates were immunoblotted for LMP-1. Results are representative of 3 LCL lines. As shown in a representative cell line in FIG. 3B, LMP-1 levels fall incrementally as a function of time after a single 10 nM dose of silvestrol. Decreases in other latent gene products involved with EBV-driven B cell transformation were observed as well. In FIG. 3D, LCL were incubated as in FIG. 3B and immunoblotted for STAT1, STAT3, AKT and NF-κB p65. Results are representative of 3 LCL lines and showed that expression of the immediate early antigen BZLF1 was unaffected.

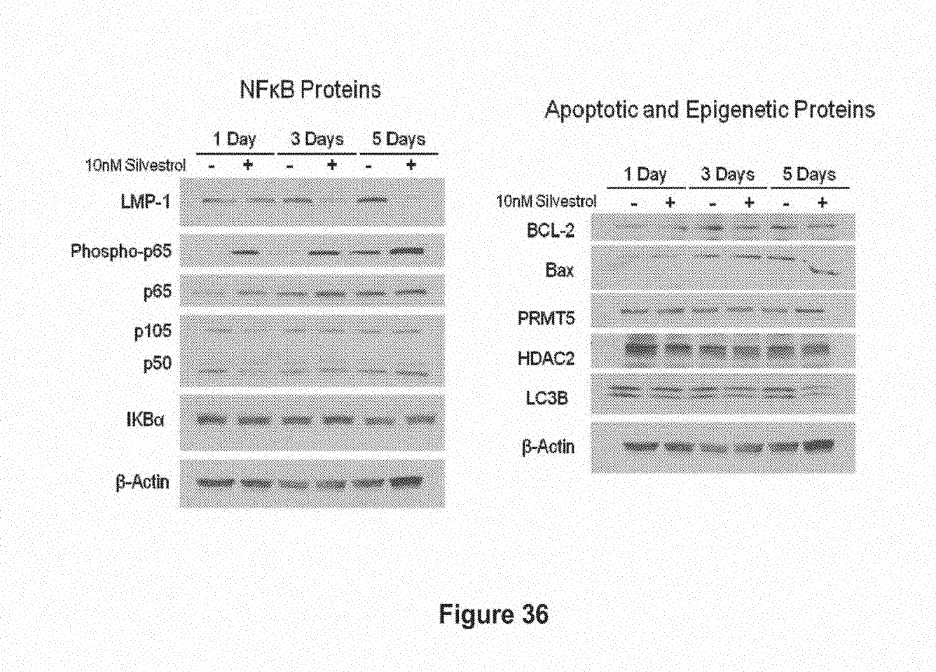

Through its cytoplasmic C-terminal-activating region 1 (CTAR1), LMP-1 constitutively activates multiple pro-survival signaling pathways including NF-κB, PI3K/AKT, and STATs 1 and 3 (Shair 2007; Gires 1999; Dawson 2003; Kung 2008; Kung 2011) similar to an external growth stimulus. Thus, LMP-1 directly and indirectly promotes tumor cell growth and survival through diverse mechanisms. To investigate the effects of silvestrol on these pathways, LCLs were incubated for 24, 72, and 120 hr with vehicle or 10 nM silvestrol, and cell extracts were analyzed by immunoblot. While total STAT1 and STAT3 levels remained unchanged, the level of phosphorylated (activated) levels of both decreased (FIG. 3D). Using the same treatment conditions, a decrease in the levels of both total Akt and its activated, phosphorylated form was observed. NF-κB p65 phosphorylation was increased with silvestrol treatment, suggesting activation, although total p65 levels were relatively unchanged. Total levels of NF-κB proteins such as p50, p105, and IκBα were unchanged (left panel), as were Bcl2 family proteins such as Bcl2, Mcl1, and Bax (right panel) (FIG. 36). Together, these data show that STAT and AKT inhibition contribute to the direct anti-tumor effects of silvestrol.

iii) Immune Effector Function is Maintained in the Presence of Silvestrol in Irradiated Co-Cultures.

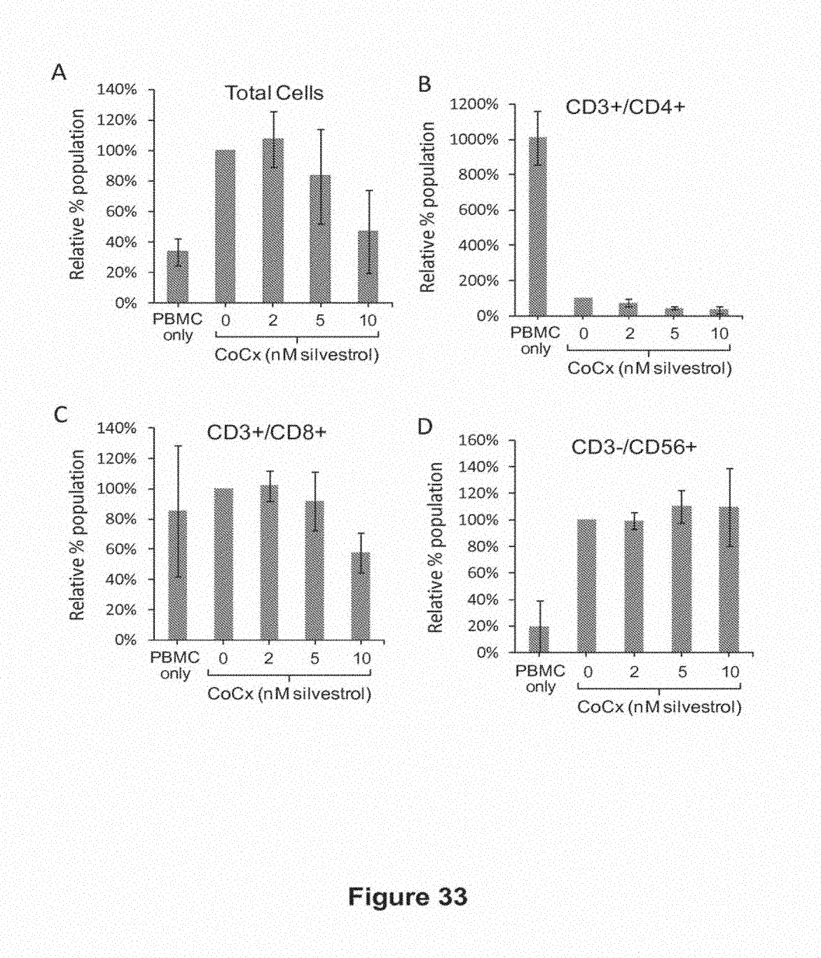

To explore the immune modulatory activity of silvestrol in EBV-LPD, in vitro co-cultures (CoCx) were used. These cultures were created by lethally irradiating LCL lines and mixing them at a ratio of 1:1 with PBMC from the same healthy donor from which the LCL was derived. The PBMC are activated and expand in response to the antigenic stimuli from the EBV-infected LCL (Rowe 1991; Baiocchi 1994; Mosier 1995). CoCx created using three LCL and their respective autologous PBMC were incubated in the presence of 10 U/mL IL-2 and treated with 0 (vehicle only), 2, 5, or 10 nM silvestrol for 14 days. Flow cytometric analysis was conducted. Cells were gated on Live events were gathered by gating on cells negative for the LIVE/DEAD stain. Data are expressed as percentage of viable population, relative to the vehicle CoCx condition, for total cells, CD3³⁰ /CD4⁺, CD3⁺/C8⁺, and CD3⁻/CD56⁺ cells Results shown are the averages from three individual CoCx. Although the total number of cells expanded in CoCx with 10 nM silvestrol was lower than in the vehicle controls, all silvestrol-treated populations had a greater total cell number than unstimulated PBMCs alone (FIG. 33A). As the irradiated LCL were absent by this time, silvestrol allowed for expansion of the normal effector population following LCL exposure. Immunophenotyping of the CoCx showed a mild decrease in the T-cell populations, both CD8³⁰ cytotoxic T cells and CD4⁺ T helper cells (FIGS. 33B and 33C; results shown relative to untreated CoCx ). Conversely, CD56⁺ natural killer cells (the majority of the expanded population) exhibited a relative increase compared to untreated CoCx (FIG. 33D).

iv) Silvestrol Leads to a Loss of LCL in Non-Irradiated Co-Cultures.

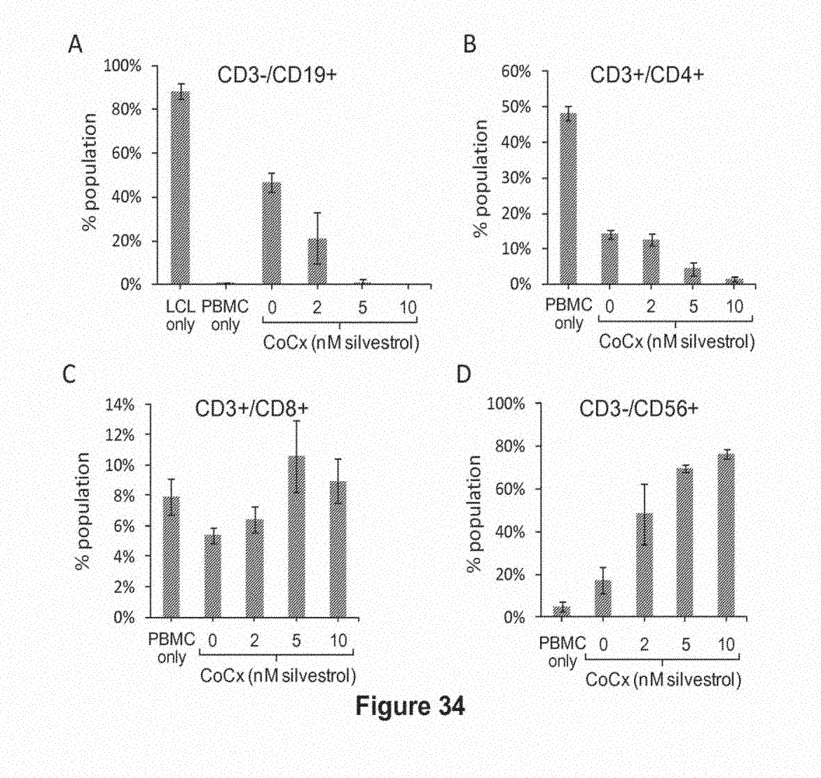

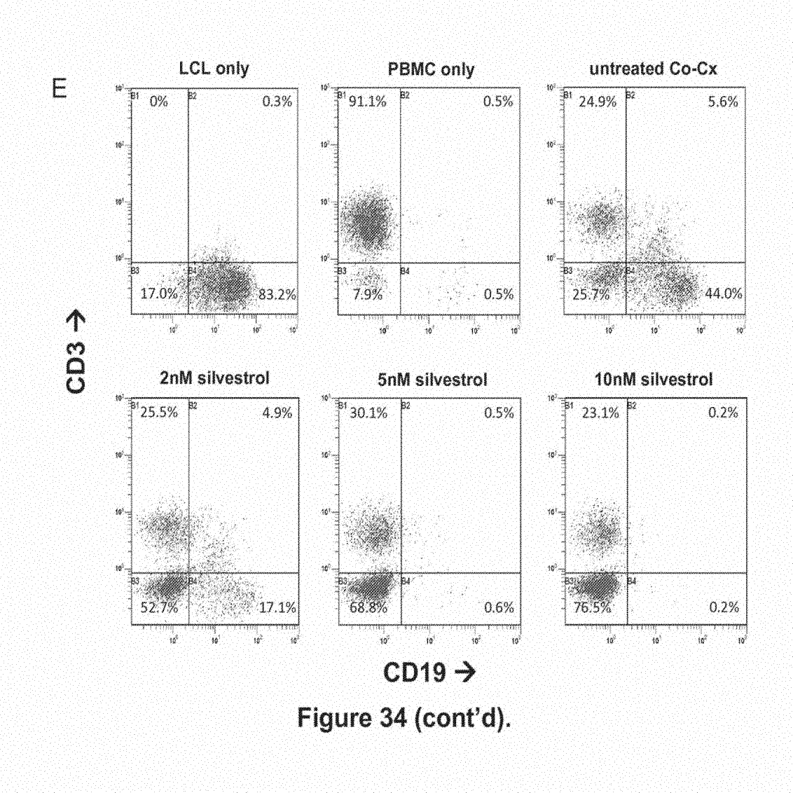

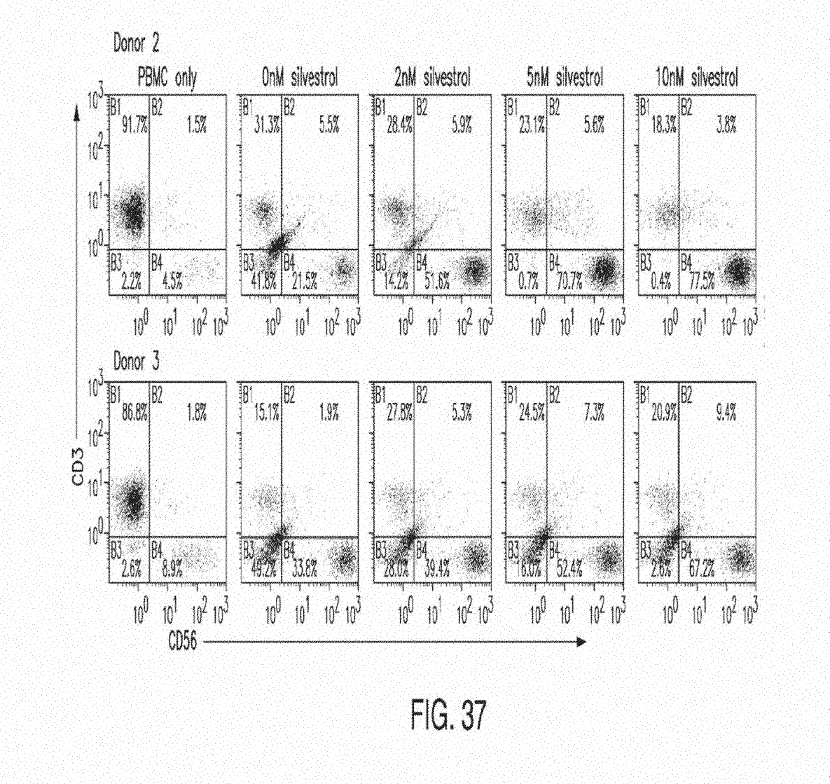

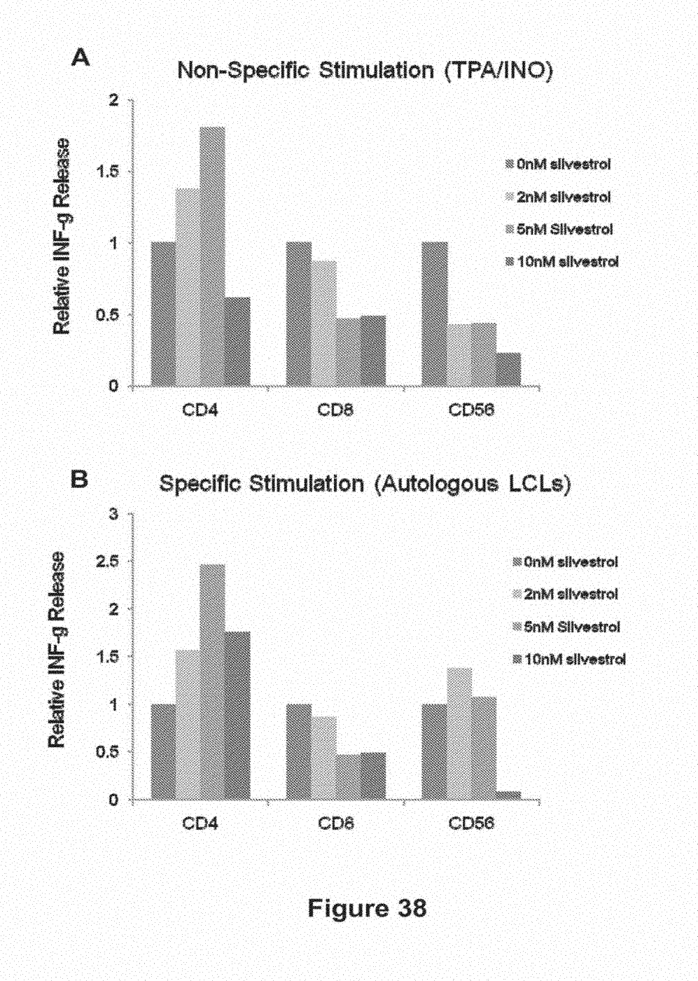

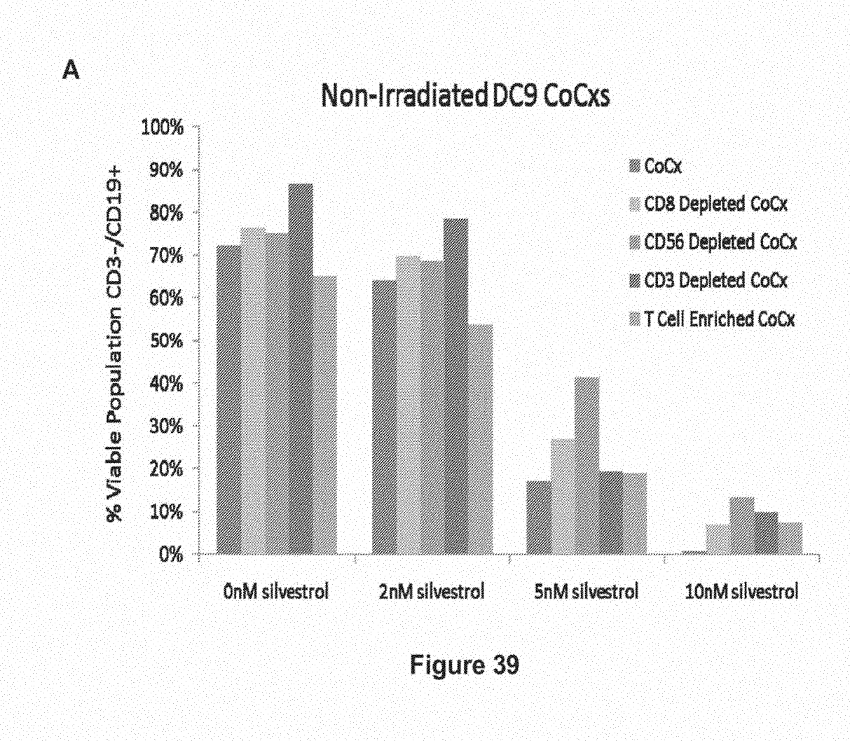

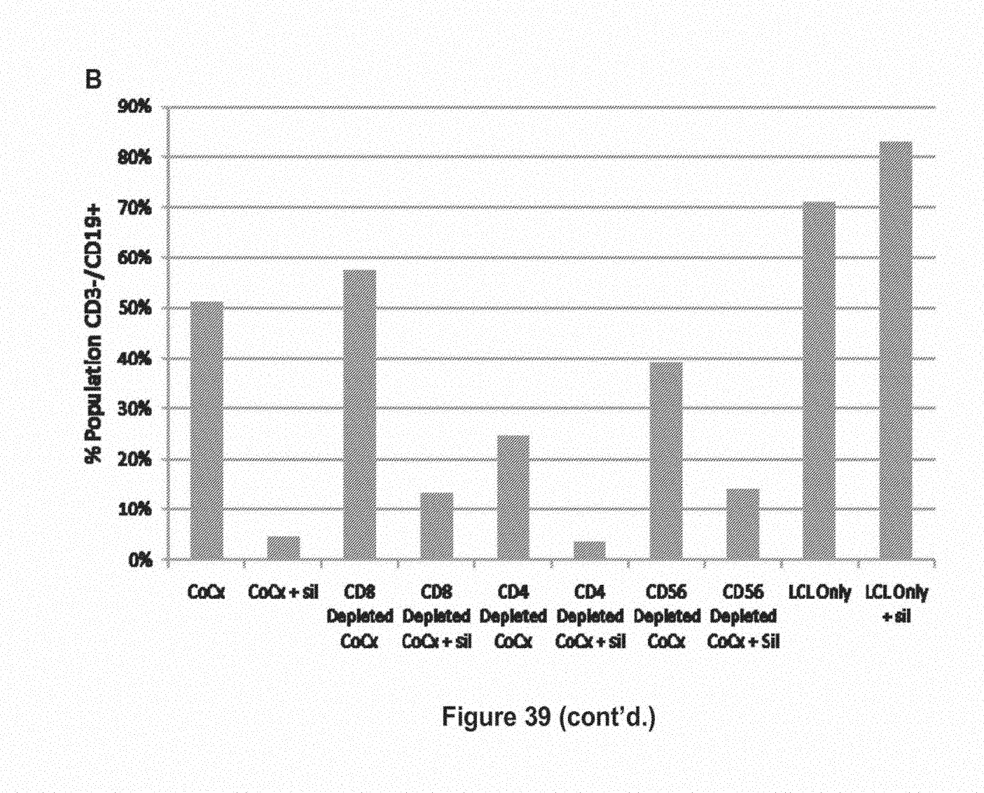

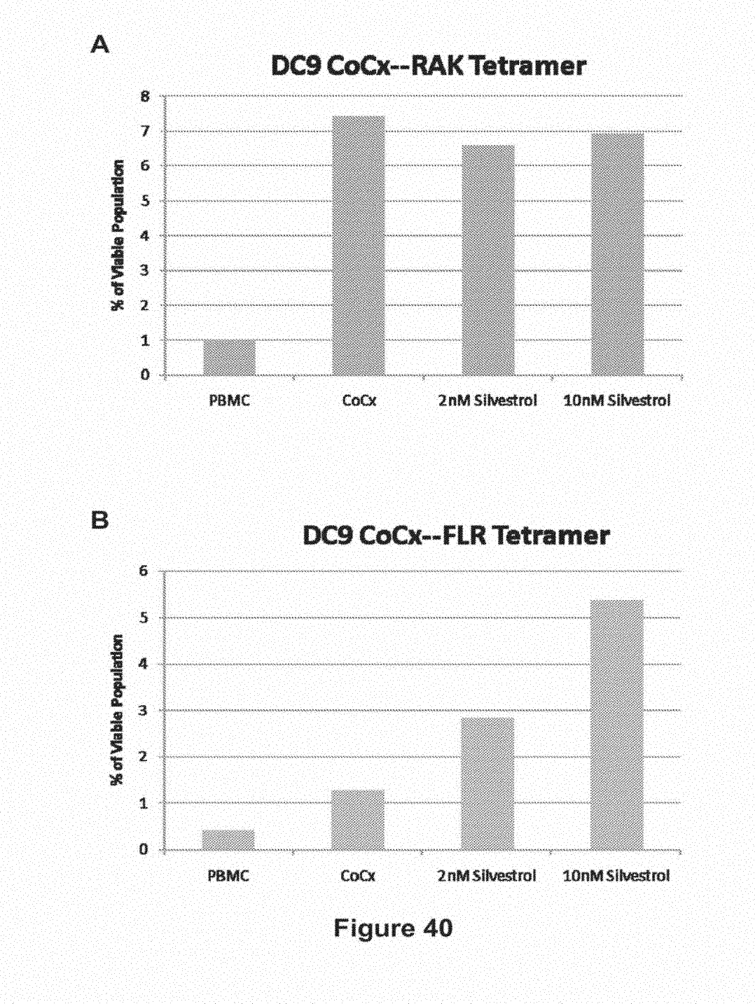

As shown above, silvestrol exhibited minimal direct anti-tumor activity on LCL at low (10 nM) doses, but allowed for the expansion of effector cell subsets exposed to irradiated LCL. It was next examined how viable, non-irradiated LCL interacted with immune effectors in the presence of silvestrol, a situation closer to conditions that occur in vivo. CoCx were incubated in the presence of 10 U/mL IL-2. Specifically, LCL from three separate donors were incubated at a ratio of 1:1 with their respective autologous PBMCs for 10 days after adding a single dose of 0, 2, 5, or 10 nM silvestrol. On day 10, flow cytometric analysis was conducted. Live events were gathered by gating on cells negative for the LIVE/DEAD stain. Data are expressed as percentage of total viable population for (A) CD3⁻/CD19⁺ LCLs; (B) CD3⁺/C4⁺ helper T cells; (C) CD3⁺/CD8⁺ CTLs; or (D) CD3⁻/CD56⁺ NK cells. Left untreated, the transformed LCL (CD3⁻/CD19³⁰ ) cells proliferated and matched the expansion of effectors lymphocyte subsets, approximately maintaining the 1:1 ratio (FIG. 34A and 34E). However, with a single addition of silvestrol,a dose-dependent ablation of viable EBV-LCL was observed (FIG. 34A). Similarly, CD4⁺ T cell percentages were reduced with increasing silvestrol concentrations (FIG. 34B). In contrast, CD8+ T cells and CD56⁺ NK cell populations expanded in the presence of silvestrol, both as percent of population and absolute numbers (FIGS. 34C-34D). Results are shown from one representative LCL; similar results were observed with LCL, and PBMC from two additional donors (FIG. 37).

Similar experiments were conducted in which CD4+ (helper T), CD8⁺ (cytotoxic T) and CD56⁺ (NK) cells were first depleted from the PBMC. These depleted CoCx all produced a similar loss of LCL, showing that each of the subsets participates in the anti-tumor activity of the autologous effector cells. FIG. 34E shows representative flow cytometry dot-plots for CD3 (y-axis) and CD19 (x-axis). LCL cells (CD3⁻/CD19⁺) appear in the bottom right quadrant of each plot. Cells were stained for CD3 (y-axis) and CD19 (x-axis) and gated on live events as LCL (CD3⁻/CD19⁺) are shown in the bottom right quadrant. All results shown are representative of three individual CoCx.

v) Silvestrol Preserves Cytotoxicity of Adaptive and Innate Immune Effectors.

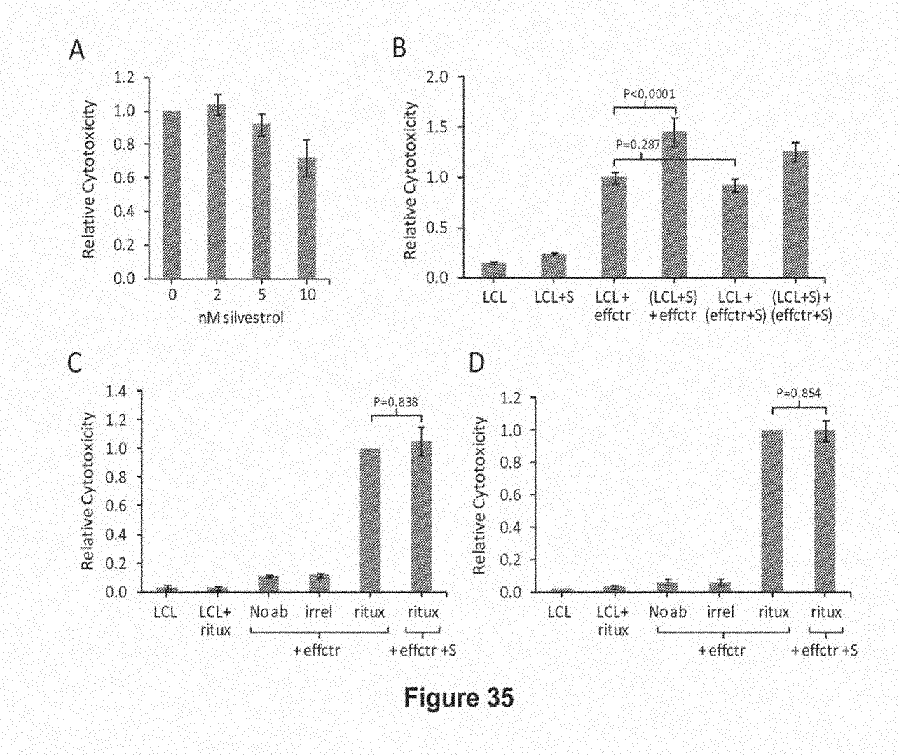

Immune cell function was then analyzed as part of the indirect anti-tumor activity of silvestrol. Cytotoxicity assays were performed. To investigate the effect of silvestrol on adaptive immunity, LCL cells were stained with the cell-tracking dye CFSE, then incubated at an effector to target ratio of 20:1 with autologous PBMC that had been cultured in the presence of irradiated LCL, with or without silvestrol, for 14 days. After a 4 hr incubation, cells were stained with the viability dye 7-AAD and washed. Cytotoxic activity against LCL targets was measured by gating on the CFSE positive events and measuring the 7-AAD positive cells (FIGS. 31-32). As shown in FIG. 35A, the flow cytometry data show that effector cells expanded in the presence of 10 nM silvestrol maintained over 70% of their cytotoxic activity against autologous LCL compared to effector cells expanded in the absence of silvestrol. Data are shown relative to the vehicle-only control and are the averages of three independent experiments. Bars show −/+ standard deviation,

Given that LCL cells were depleted and effector cell cytotoxicity was maintained in non-irradiated co-cultures, whether silvestrol lowers the apoptotic threshold of the LCL tumor target was examined. PBMC were co-cultured with irradiated LCL in the absence of silvestrol for 14 days. These expanded effectors and fresh LCL each were separately incubated with silvestrol or vehicle control for 18 hr and washed. Cells were then combined, and after 4 hr incubation, cytotoxicity assays were performed as above. Silvestrol-treated effectors showed no decrease in their ability to kill targets compared to the untreated effectors (p=0.287; FIG. 35B). Data are shown relative to the vehicle-only control and are the averages of three independent experiments. Bars show −/+ standard deviation. However, LCL target cells pre-treated with silvestrol for 18 hr were more efficiently killed compared to untreated targets (p<0.0001). These data indicate that silvestrol, even at minimally cytotoxic concentrations, significantly increases the sensitivity of tumor cells to effector cell-mediated killing.

Next, antibody-dependent cell-mediated cytotoxicity (ADCC) assays were utilized to measure the innate immune response of NK cells to LCL in the presence of silvestrol. Effector cells were expanded in the presence of irradiated LCL, with or without 10 nM silvestrol, for 14 days prior to being enriched for NK cells. Fresh CFSE-labeled LCL targets and autologous NK cells were incubated with the monoclonal antibodies rituximab (anti-CD20, expressed on LCL) or herceptin (anti-HER2, not expressed on immune cells). After 4 hr, ADCC was evaluated using CFSE and 7-AAD staining as described above. Specifically, cytotoxicity was measured by CFSE⁺/7AAD⁺ events. Data are shown relative to the positive control (effectors+targets+ritux) and are the averages of three individual experiments. Bars show −/+ standard deviation. As shown in FIG. 35C, rituximab-dependent cytotoxic activity of the NK cells was similar between Effectors expanded in the presence vs. the absence of silvestrol (p=0.838).

To determine the acute effect of silvestrol on ADCC, fresh (non-autologous) NK cells were obtained from enriched leukocyte products, incubated 18 hr with or without 10 nM silvestrol, then washed and mixed with CFSE-stained LCL. As shown in FIG. 35D, silvestrol pre-incubation produced no observable effect on ADCC activity of freshly isolated NK cells (p=0.854). Data are shown relative to the positive control (effectors+targets+ritux) and are the averages of three individual experiments. Bars show −/+ standard deviation.

vi) In Vivo Efficacy of Silvestrol in the Hu-PBL SCID Model of EBV-LPD.

The hu-PBL SCID model has been used to study the effect of compounds on spontaneously derived EBV-driven lymphomas (Fuzzati-Armentero 1998) PBMC from a healthy EBV-positive donor were injected into a group of SCID mice that had been pretreated with anti-asialo (GM1) to deplete murine NK cells. Mice were randomized (n=14/group) and treatment with 1.5 mg/kg silvestrol or vehicle control every 48 hr began two weeks after engraftment. Two weeks after treatment began (four weeks post-engraftment), ELISA was performed to evaluate human IgG. All mice (n=28) were shown to produce human IgG, demonstrating engraftment. As shown in FIG. 3, silvestrol did not affect the production of human IgG by the xenografted B-lymphocytes in the hu-PBL-SCID mode (liars show −/+ standard deviation), indicating that silvestrol allows the preservation of adaptive humoral immune surveillance. At 4 and 8 weeks post-engraftment, flow cytometry analysis was performed on spleen cells from two mice in each group. Mice in the vehicle control group showed substantial EBV-LPD infiltration of the spleen, whereas mice from the silvestrol-treated group showed no tumor burden; furthermore, by eight weeks post-engraftment spleens from each group showed clear differences in size (FIG. 25A). Spleens were also obtained from mice as the disease progressed and euthanasia criteria were met. Mice in the vehicle control group showed significantly enlarged spleens relative to silvestrol-treated mice (mean spleen weight 421.6 mg vs. 72.9 mg for silvestrol-treated mice after completion of the experiment at day 140; p≧0.0004) (FIG. 25B). During the 140-day experiment, the health of the mice was tracked by body weight. Weights of all mice were checked twice weekly. Mice treated with silvestrol did not show observable ill effects from the long-term treatment, and appeared to gain weight at a faster pace when compared to the vehicle treated mice (FIG. 25A). Finally, as shown in FIG. 27, a Kaplan-Meier analysis of overall survival shows that silvestrol-treated mice showed significantly improved survival compared to mice treated with vehicle control (7/7 silvestrol treated mice vs. 2/9 control mice alive at day 140, p<0.001). Silvestrol-treated mice were examined at the end of the study and exhibited no signs of disease as determined by flow cytometry of spleen cells.

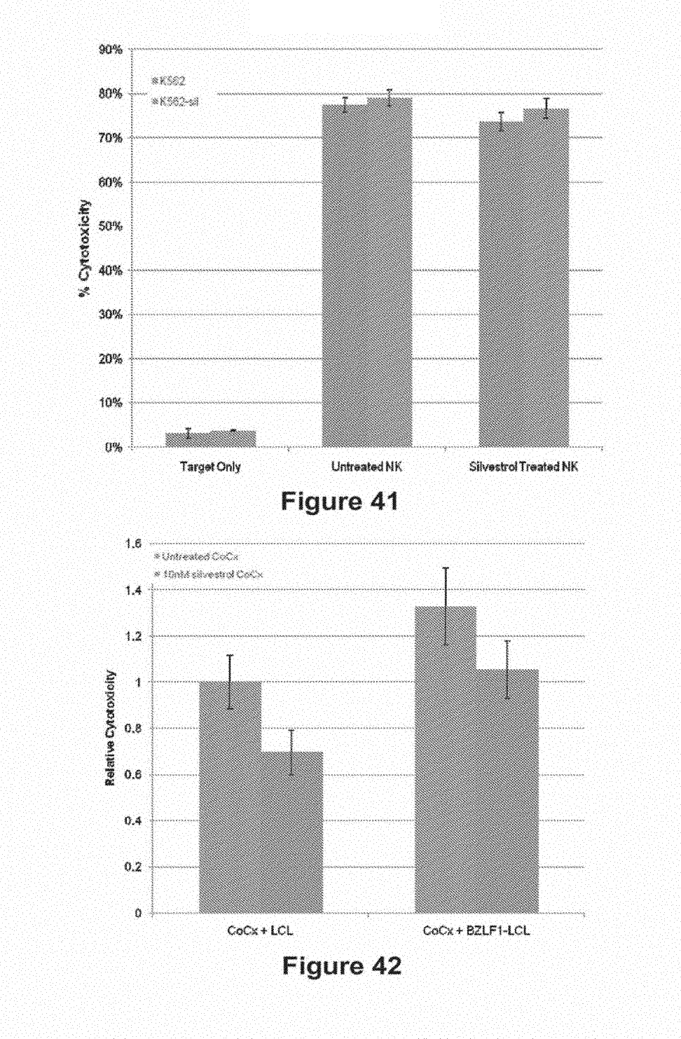

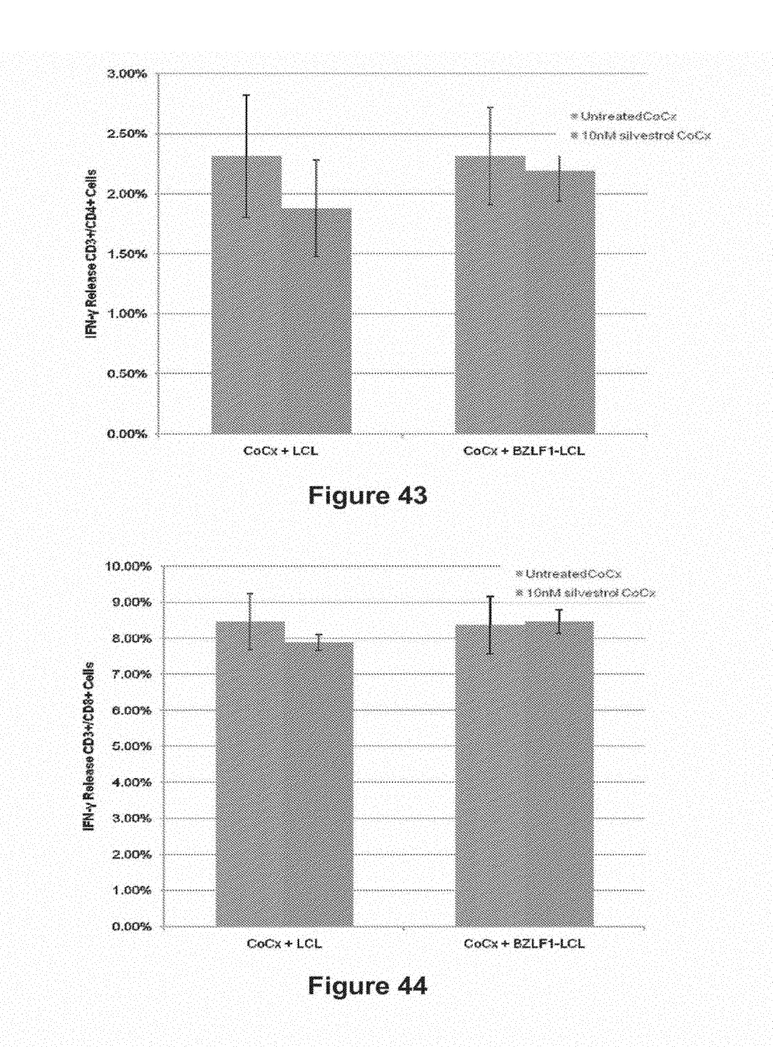

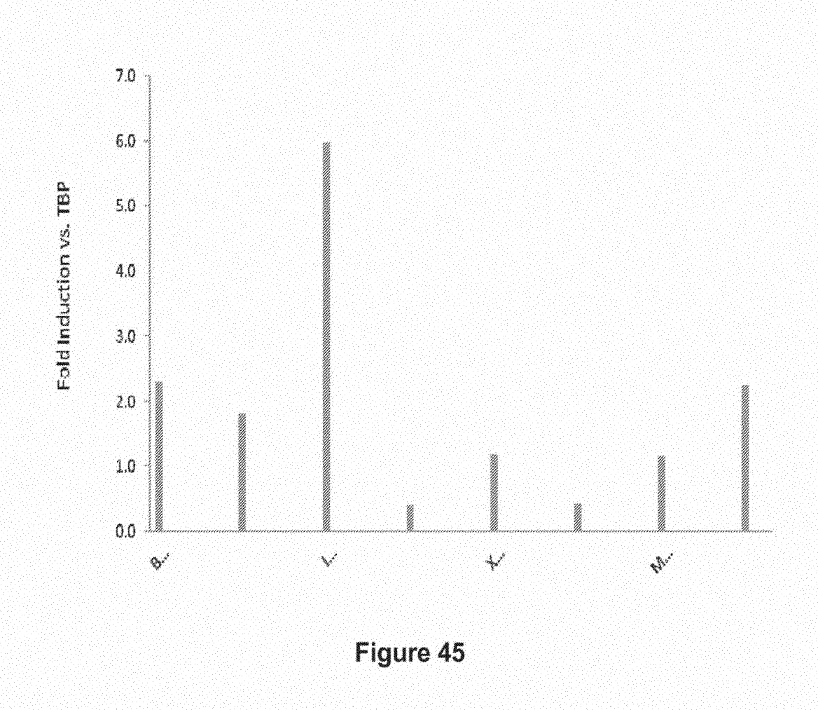

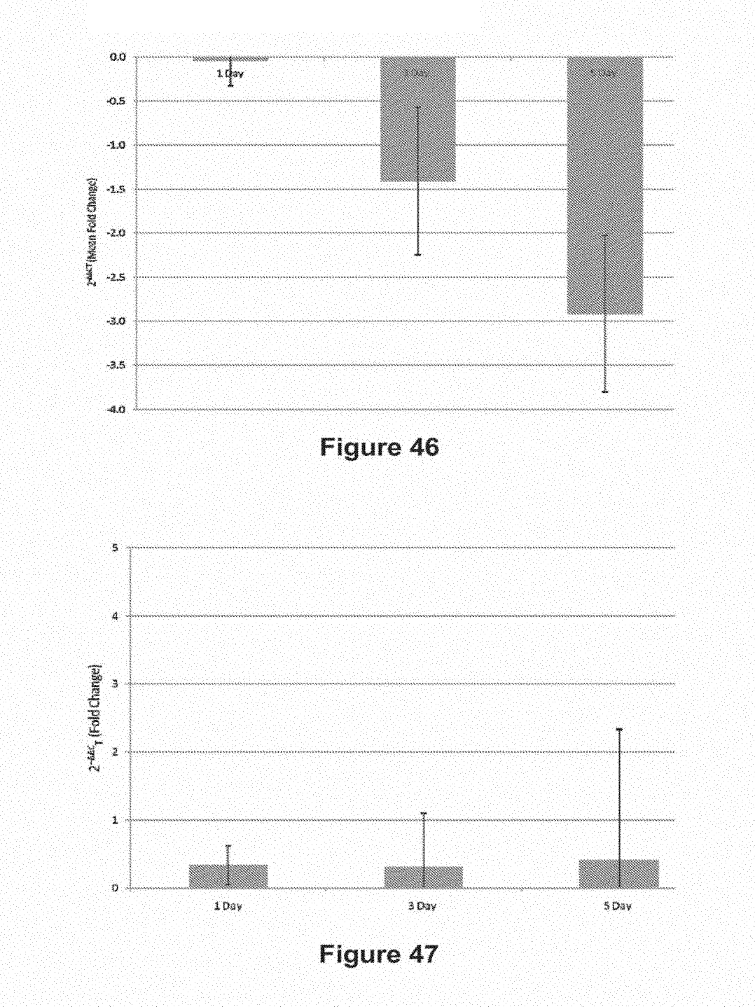

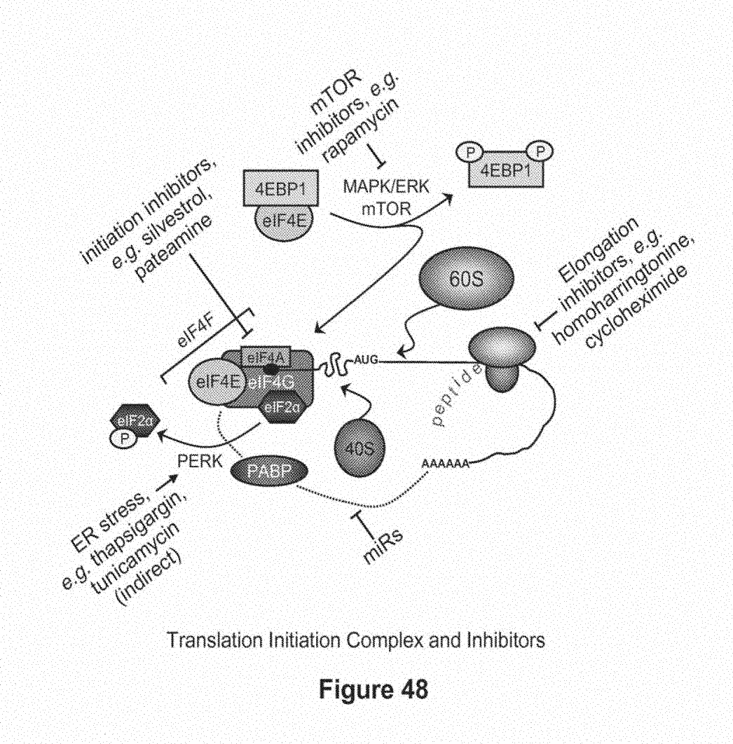

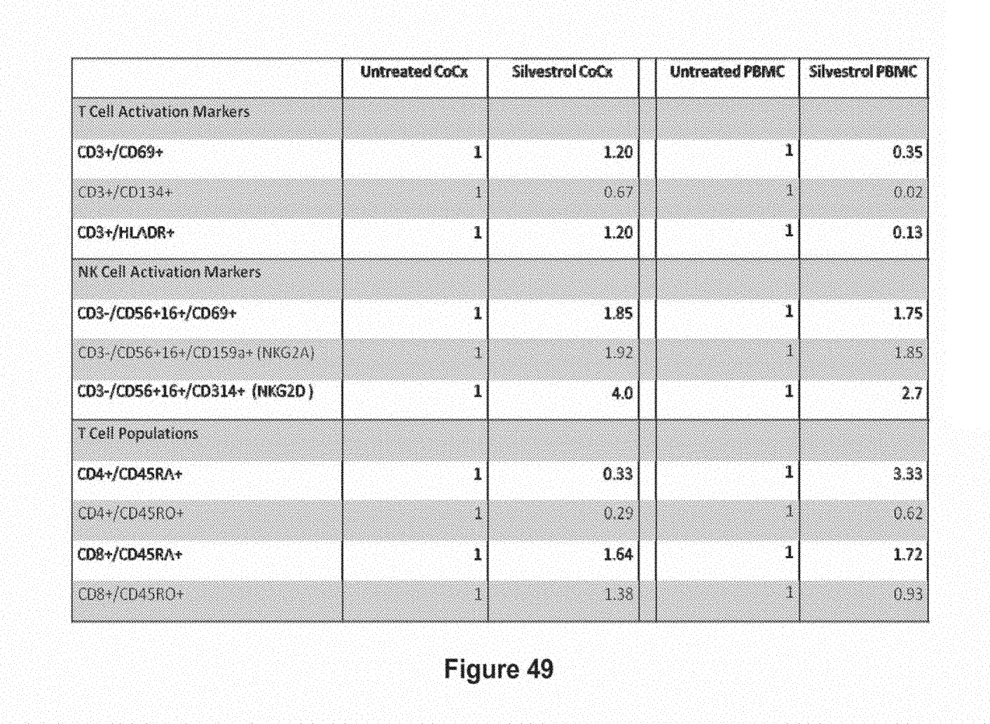

FIG. 41 presents data regarding NK cytotoxicity of K562 cells. The data indicate that the direct killing of K562 cells by NK cells was not adversely affected by the presence of silvestrol (10 nM). FIG. 42 presents data regarding the cytotoxicity of LCLs pretreated with BZLF1. The data show that the direct killing of autologous EBV+ LCLs (tumor) by EBV-specific CTLs was not adversely affected by the presence of silvestrol (10 nM). BZLF1 antigen pulsed LCLs promoted enhanced killing in untreated and silvestrol-treated CTLs. FIG. 43 presents data relating to CD4 IFNgamma release with LCL pretreated with BZLF1. The data show that the IFNgamma release of CD4⁺ T cells was not adversely affected by the presence of silvestrol (10 nM). FIG. 44 presents data relating to CD4 IFNgamma release with LCL pretreated with BZLF 1. The data show that the IFNgamma release of CD4³⁰ T cells was not adversely affected by the presence of silvestrol (10 nM) in LCL pretreated with BZLF1. FIG. 45 presents data relating to interleukin-6 induction by silvestrol. The real-time RT-PCR for NF-κB targets show that interleukin-6 was induced by silvestrol (10 nM) in LCL. The data show that the IFNgamma release of CD4³⁰ T cells was not adversely affected by the presence of silvestrol (10 nM) in LCL pretreated with BZLF1. FIG. 46 presents data relating to a change in LMP-1 expression following silvestrol treatment. The data show that silvestrol treatment decreased the expression of the EBV LMP-1 oncogene transcript. Three cell lines were examined on days 1, 3, and 5 following treatment. FIG. 47 presents data relating to a change in EBNA1 expression following silvestrol treatment. Three cell lines were examined on days 1, 3, and 5 following treatment.

c) Discussion

The oncogenic virus EBV is associated with a broad spectrum of benign and malignant diseases (Rickinson 1986). Healthy individuals mount an efficient immune response to EBV infection, controlling the proliferation of latently infected cells. However in immunocompromised persons, including transplant, HIV-AIDS, and elderly patients, reactivation of the virus can cause transformation of infected B lymphocytes, leading to aggressive LPD (Sato 1989; Weinstock 2006). Current therapies for LPD often lead to further immune suppression and subsequent development of life-threatening opportunistic infections and viral reactivation. Thus, an ideal agent to treat patients with EBV LPD possesses direct anti-tumor activity while preserving the host anti-tumor immune function. Here it is shown that silvestrol can be such an agent, and provided is a characterization of the unique anti-tumor and immune-potentiating properties of silvestrol using in vitro and in vivo models of EBV LPD.

The direct anti-proliferative activity of silvestrol was associated with a loss of LMP-1 expression. Drugs and cytotoxic T cell preparations directly targeting LMP-1 have been shown to prevent metastasis, promote apoptosis and enhance radiosensitivity, identifying LMP-1 as a potential therapeutic target for EBV-driven malignancies (Gottschalk 2003; Kenney 2001; Mei 2007; Hannigan 2010; Yang 2010.) Silvestrol appears to down-modulate LMP-1 more substantially compared to latent gene products EBNA 2 and 3C. The ablation of LMP-1 protein has been shown to interfere with several direct downstream signaling pathways including Akt (Kenney 2001) and STAT1 and STAT3 (Gires 1999; Kung 200) and the results with silvestrol support these findings. In addition, enhanced sensitivity of LCL targets that were pretreated with low-dose silvestrol when incubated with effector cells was found. The classical chemotherapeutic agent's paclitaxel, cisplatin, and doxorubicin sensitize several types of solid tumor cells to cytotoxic T lymphocytes by increasing tumor cell permeability to granzyme B (Ramakrishnan 2010).

As described herein, silvestrol maintains adaptive immune effectors (EBV-specific cytotoxic T cells) and innate immune effectors (natural killer cells) using effectors expanded with irradiated LCLs. Effector cell function was maintained in co-cultures with viable LCL in the presence of 10 nM silvestrol. (See, e.g., FIGS. 28-30). This concentration of silvestrol had minimal effects on LCL by itself, and in untreated co-cultures, autologous PBMC were unable to expand sufficiently to eliminate the LCL tumor targets. The combination of 10 nM silvestrol with autologous PBMC led to expansion of active T and NK cell populations, production of IFNgamma, and elimination of the LCL. These results show that the indirect effects of silvestrol on tumor cells via innate and adaptive immune components can be at least as important as its direct effects. As nearly all in vivo studies to date with silvestrol have used tumor xenegrafts in immune deficient mice, this information was previously unknown.

Chemotherapy also has a deleterious effect on NK cell-mediated ADCC (Saijo 1982; Kurai 2007). Because NK cells were the main lymphocyte population to expand from the co-cultures with non-irradiated LCL, the consequences of silvestrol on innate immune function was examined. These experiments showed no effect of silvestrol on NK cell ADCC activity, either when NK cells were expanded in the presence of silvestrol or when silvestrol was present during the ADCC assay.

Finally, the in vivo studies using the hu-PBL SCID mouse model provided insight into the efficacy of silvestrol in treating EBV-positive LPD. The pre-clinical model is an aggressive EBV-induced lymphoma model that progresses rapidly with the onset of disease (Mosier. 1992).

All of the compositions and/or methods disclosed and claimed herein can be made and executed without undue experimentation in light of the present disclosure. It will be apparent to those skilled in the art that various modifications and variations can be made in the present invention without departing from the scope or spirit of the invention.

More specifically, certain agents which are both chemically and physiologically related can be substituted for the agents described herein while the same or similar results can be achieved. All such similar substitutes and modifications apparent to those skilled in the art are deemed to be within the spirit, scope and concept of the invention as defined by the appended claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

Adams, T E, El Sous, M, Hawkins, B C, et al. Total synthesis of the potent anticancer Aglaia metabolites (−)-silvestrol and (−)-episilvestrol and the active analogue (−)-4′-desmethoxyepisilvestrol. J Am Chem Soc 2009; 131: 1607-16

Alinari L, Edwards R B, Prince C J, et al. Targeting the Cyclin D-CDK4/6-Rb Axis in Mantle Cell Lymphoma with the Novel Translation Inhibitor Silvestrol. Blood. 2011; 118:3498a.

Alinari L, Yu B, Christian B A, et al. Combination anti-CD74 (milatuzumab) and anti-CD20 (rituximab) monoclonal antibody therapy has in vitro and in vivo activity mantle cell lymphoma. Blood. 2011; 117:4530-4541.

Baiocchi R A, Caligiuri M A. Low-dose interleukin 2 prevents the development of Epstein-Barr virus (EBV)-associated lymphoproliferative disease in scid/scid mice reconstituted i.p. with EBV-seropositive human peripheral blood lymphocytes. Proc. Natl Acad Sci USA. 1994; 91:5577-5581.

Baumann, B, Bohnenstengel, F, Siegmund, D, et al. Rocaglamide derivatives are potent inhibitors of NF-kappa B activation in T-cells. J Biol Chem 2002; 277: 44791-800

Bichi R, Shinton, S A Martin, E S, et al. Human chronic lymphocytic leukemia modeled in mouse by targeted TCL1 expression. Proc Natl Acad Sci USA 2002; 99:6955-60.

Bohnenstengel, F I, Steube, K G, Meyer, C, et al. Structure activity relationships of antiproliferative rocaglamide derivatives from Aglaia species (Meliaceae). Z Naturforsch [C] 1999; 54: 55-60

Bohnenstengel, F I, Steube, K G, Meyer, C, et al. 1H-cyclopenta[b]benzofuran lignans from Aglaia species inhibit cell proliferation and alter cell cycle distribution in human monocytic leukemia cell lines. Z Naturforsch [C] 1999; 54: 1075-83.

Bordeleau, M E, Robert, F, Gerard, B, et al. Therapeutic suppression of translation initiation modulates chemosensitivity in a mouse lymphoma model. J Clin Invest 2008; 118: 1-11

Cencic, R, Carrier, M, Galicia-Vazquez, G. et al. Antitumor activity and mechanism of action of the cyclopenta[b]benzofuran, silvestrol. PLoS One 2009; 4: e5223

Cencic, R, Carrier M, Tmkus A, Porco J A, Jr., Minden M. Pelletier J. Synergistic effect of inhibiting translation initiation in combination with cytotoxic agents in acute myelogenous leukemia cells. Leuk Res. 2010; 34:535-541.

Chen H. Hutt-Fletcher L, Cao L, Hayward S D. A positive autoregulatory loop of LMP1 expression and STAT activation in epithelial cells latently infected with Epstein-Barr virus. J Viral. 2003; 77:4139-4148.

C W, Tramountanis G, Eliopoulos A G, Young L S. Epstein-Barr virus latent membrane protein 1 (LMP1) activates the phosphatidylinositol 3-kinase/Akt pathway to promote cell survival and induce actin filament remodeling. J Biol Chem. 2003; 278:3694-3704

El Sous, M, Khoo, M, Holloway, G, et al. Total synthesis of (−)-episilvestrol and (−)-silvestrol. Angew. Chemie Int. Ed. 2007; 46: 7835-8

Fuzzati-Armentero M T, Duchosal M A. hu-PBL-SCID mice: an in vivo model of Epstein-Barr virus-dependent lymphoproliferative disease. Histol Histopathol. 1998; 13:155-168.

Gerard, B, Cencic, R, Pelletier, J and Porco, J. Enantioselective synthesis of the complex rocaglate (−)-silvestrol. Angew. Chemie Int. Ed. 2007; 46: 7831-4

Gires O, Kohlhuber F, Kilger E, et al. Latent membrane protein 1 of Epstein-Barr virus interacts with JAK3 and activates STAT proteins. EMBO J. 1999; 18:3064-3073.

Gires O, Zimber-Strobl U, Gonnella R, et al. Latent membrane protein 1 of Epstein-Bart virus mimics a constitutively active receptor molecule. EMBO J. 1997; 16:6131-6140.

Godoy-Ramirez K, Makitalo B, Thorstensson R, Sandstrom E, Biberfeld G, Gaines H. A novel assay for assessment of HIV-specific cytotoxicity by multiparameter flow cytometry. Cytometry A. 2005; 68:71-80.

Gottschalk S, Edwards O L, Sili U, et al. Generating CTLs against the subdominant Epstein-Barr virus LMP1 antigen for the adoptive immunotherapy of EBV-associated malignancies. Blood. 2003; 101:1905-1912.

Grever. Direct anti-tumor activity in CLL, ALL models. Blood; 2009.

Hande, K R, Etoposide: four decades of development of a topoisomerase II inhibitor. Eur J Cancer 1998; 34: 1514-21

Hannigan A, Wilson J B. Evaluation of LMP1 of Epstein-Barr virus as a therapeutic target by its inhibition. Mol Cancer. 2010; 9:184.

Helguera G, Rodriguez J A, Luria-Perez R, et al. Visualization and quantification of cytotoxicity mediated by antibodies using imaging flow cytometry. J Immunol Methods. 2011; 368:54-63.

Hollingshead, M, Roberson, J, Decker, W, et al. In vivo drug screening applications of HIV-infected cells cultivated within hollow fibers in two physiologic compartments of mice. Antiviral Res 1995; 28:265-79.

Hussain, S R, Cheney, C M, Johnson, A J, et al. Mcl-1 is a relevant therapeutic target in acute and chronic lymphoid malignancies: down-regulation enhances rituximab-medicated apoptosis and complement-dependent cytotoxicity. Clin Cancer Res 2007; 13;2144-50.

Hwang, B Y, Su, B N, Chai, H, et al. Silvestrol and episilvestrol, potential anticancer rocaglate derivatives from Aglaia silvestris, J Org Chem 2004; 69: 3350-8; ibid. 6156

Kenney J L, Guinness M E, Reiss M, Lacy J. Antisense to the Epstein-Barr virus (EBV)-encoded latent membrane protein 1 (LMP-1) sensitizes EBV-immortalized B cells to transforming growth factor-beta and chemotherapeutic agents. Int J Cancer. 2001; 91:89-98.

Kieff E, Rickinson A B. Epstein-Barr Virus. In: Knipe DM HP ed. Fields Virology. Vol. 75:2575-2678. Philadelphia: Lippincott Williams & Wilkins; 2001.

Kim, S. Salim, A A, Swanson, S M and Kinghorn, A D. Potential of cyclopenta[b] benzofurans from Aglaia species in cancer chemotherapy. Anticancer Agents Med Chem 2006; 6: 319-45

Kim, S. Hwang, B Y, Su, B N, et al. Silvestrol, a potential anticancer rocaglate derivative from Aglaia foveolata, induces apoptosis in LNCaP cells is through the mitochondrial/apoptosome pathway without activation of executioner caspase-3 or -7. Anticancer Res 2007; 27: 2175-83

King, M L, Chiang, C C, Ling, H C, et al. X-ray crystal structure of rocaglamide, a novel antileukemic 1H-cyclopenta[b]benzofuran from Aglaia elliptifolia, J. Chem. Soc. Chem. Commun. 1982; 1150-1

Kinghorn Structurally unique natural product isolated from the Indonesian plant Agllaia foveolata J. Org Chem 2004; 69: 3350-3358.

Kung C P, Meckes D G, Jr., Raab-Traub N. Epstein-Barr virus LMP1 activates EGFR, STAT3, and ERK through effects on PKCdelta. J Virol. 2011; 85:4399-4408.

Kung C P, Raab-Traub N. Epstein-Barr virus latent membrane protein 1 induces expression of the epidermal growth factor receptor through effects on Bcl-3 and STAT3. J Virol. 2008; 82:5486-5493.

Kurai J, Chikumi H, Hashimoto K, et al. Antibody-dependent cellular cytotoxicity mediated by cetuximab against lung cancer cell lines. Clin Cancer Res. 2007; 13;1552-1561.

Lee, K-H and Xiao, Z. In: Cragg, G M, Kingston, DGI and Newman, D J Eds., Anticancer Agents from Natural Products. Boca Raton, Fla., CRC/Taylor & Francis, 2005; 71-87

Lindqvist, L and Pelletier, J. Inhibitors of translation initiation as cancer therapeutics; 2009. 9:1709-1722.

Lowe et al. Model for studying the role of genes in chemoresistance. US Publication US2009/0186839, Jul. 23, 2009.

Lucas, D M, Edwards, R B, Lozanski, G, et al. The novel plant-derived agent silvestrol has B-cell selective activity in chronic lymphocytic leukemia and acute lymphoblastic leukemia in vitro and in vivo. Blood 2009; 113: 4656-66

Mei Y P, Zhou J M, Wang Y, et al. Silencing of LMP1 induces cell cycle arrest and enhances chemosensitivity through inhibition of AKT signaling pathway in EBV-positive nasopharyngeal carcinoma cells. Cell Cycle; 2007; 6:1379-1385.

Meurer-Grimes et al. Therapeutic compounds and methods, U.S. Pat. No. 6,710,075, Mar. 23, 2004.

Mi, Q, Kim, S, Hwang, B Y, et al. Silvestrol regulates G2/M checkpoint genes independent of p53 activity. Anticancer Res 2006; 26: 3349-56

Mosier D E, Picchio O R, Baird S M, Kobayashi R. Kipps T J. Epstein-Barr virus-induced human B-cell lymphomas in SCID mice reconstituted with human peripheral blood leukocytes. Cancer Res. 1992; 52;5552s-5551s

Najjar I, Baran-Marszak F, Le Clorennec C, et al. Latent membrane protein 1 regulates STAT1 through NF-kappaB-dependent interferon secretion in Epstein-Barr virus-immortalized B cells. J Virol. 2005; 79:4936-4943.

Park S, Lee J, Ko Y H, et al. The impact of Epstein-Barr virus status on clinical outcome in diffuse large B-cell lymphoma. Blood. 2007; 110:972-978.

Patton J T, Lustberg, M E, Garman, S L, Kinghorn, A D, Pan, L, Lucas, D M, Grever, R and Baiocchi, R A. Silvestrol modulates direct anti-tumor activity against Epstein-Barr Virus (EBV)-associated lymphomas while sparing innate and antigen specific adaptive immunity. Blood,

Pelletier, J. Inhibits translation initiation by promoting aberrant interaction of eIF4A with capped mRNA; J Clin Invest; 2008

Pelletier et al. Chemotherapeutic agents for inhibition of protein translation. US Publication No. US2009/0118362, May 7, 2009.

Proksch, P, Giaisi, M, Treiber, M K, et al. Rocaglamide derivatives are immunosuppressive phytochomicals that target NF-AT activity in T cells. J Immunol 2005; 174: 7075-84.

Ramakrishnan R, Assudani D, Nagaraj S, et al. Chemotherapy enhances tumor cell susceptibility to CTL-mediated killing during cancer immunotherapy in mice. J Clin Invest. 2010; 120:1111-1124.

Rickinson A B ed The Epstein-Barr Virus: Recent Advances. In: Epstein MAA, B. G. ed. London: Heinemann Med. Books; 1986.

Rose, W C, Schurig, J E and Meeker, J B. Correlation of in vitro cytotoxicity with preclinical in vivo antitumor activity. Anticancer Res 1988; 8: 355-67

Rowe M, Young L S, Crocker J, Stokes H, Henderson S, Rickinson A B. Epstein Barr virus (EBV)-associated lymphoproliferative disease in the SCID mouse model: implications for the pathogenesis of EBV-positive lymphomas in man. J Exp Med. 1991; 173:147-158

Saijo N, Shimizu E, Shibuya M, et al. Effect of chemotherapy on natural-killer activity and antibody-dependent cell-mediated cytotoxicity in carcinoma of the lung. Br J Cancer, 1982; 46:180-189.

Salim, A A. Chai, H B, Rachman, I, et al. Constituents of the leaves and stem bark of Aglaia foveolata. Tetrahedron 2007; 63: 7926-34

Saradhi U V, Gupta S V, Chiu M, et al. Characterization of silvestrol pharmacokinetics in mice using liquid chromatography-tandem mass spectrometry, AAPS J. 2011; 13:347-356.

Sato H, Takimoto T, Hatano M, Pagano J S, Raab-Traub N. Epstein-Barr virus with transforming and early antigen-inducing ability originating from nasopharyngeal carcinoma: mapping of the viral genome. J Gen Virol. 1989; 70 (Pt 3):717-727.

Shair K H, Bendt K M, Edwards R H, Bedford E C, Nielsen J N, Raab-Traub N. EBV latent membrane protein 1 activates Akt, NFkappaB, and Stat3 in B cell lymphomas. PLoS Pathog. 2007; 3:e166

Su, B N, Chai, H, Mi, Q, et al. Activity-guided isolation of cytotoxic constituents from the bark of Aglaia crassinervia collected in Indonesia. Bioorg Med Chem 2006; 14: 960-72

van Maanen, J M S, Retel, J, de Vries, J and Pinedo, H M. Mechanism of action of the antitumor drug etoposide: a review. J. Nat. Cancer Inst. 1988; 80; 1526-33

Weinstock D M, Ambrossi G G, Brennan C, Kiehn T E, Jakubowski A. Preemptive diagnosis and treatment of Epstein-Barr virus-associated post transplant lymphoproliferative disorder after hematopoietic stem cell transplant; an approach in development. Bone Marrow Transplant. 2006; 37:539-546.

Wu, T S, Liou, M J, Kuoh, C S, et al. Cytotoxic and antiplatelet aggregation principles from Aglaia elliptifolia. J Nat Prod. 1997; 60: 606-8

Yang L, Lu Z, Ma X, Cao Y. Sun L Q. A therapeutic approach to nasopharyngeal carcinomas by DNAzymes targeting EBV LMP-1 gene. Molecules. 2010; 15:6127-6139.

Zhu, J Y, Lavrik, I N, Mahlknecht, U, et al. The traditional Chinese herbal compound rocaglamide preferentially induces apoptosis in leukemia cells by modulation of mitogen-activated protein kinase activities. Int J Cancer 2007; 121: 1839-46

1-128. (canceled)

129. A composition comprising: silvestrol or a silvestrol analog; a vaccine; and a pharmaceutically acceptable carrier.

130. The composition according to claim 129, wherein the vaccine comprises a preventative vaccine comprising tumor cell vaccines, antigen vaccines, dendritic cell vaccines, DNA vaccines, or vector-based vaccines.

131. The composition according to claim 129, wherein the vaccine comprises a therapeutic vaccine comprising tumor cell vaccines, antigen vaccines, dendritic cell vaccines, DNA vaccines, or vector-based vaccines

132. The composition according to claim 130, wherein the vaccine comprises an antigen vaccine.

133. The composition according to claim 129, comprising a silvestrol analog having the structure: 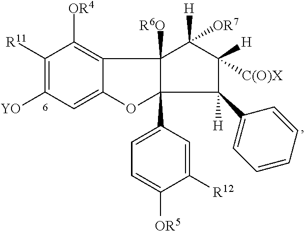
wherein each R⁴-R¹⁰ is independently selected from the group consisting of hydrogen, optionally substituted alkyl, optionally substituted acyl, optionally substituted aryl, optionally substituted arylalkyl, optionally substituted cycloalkylalkyl, optionally substituted arylacyl, optionally substituted cycloalkylacyl and a C-1 linked saccharide;
X is OR⁸ or NR9R10;
R¹¹ and R¹² are each independently hydrogen, or OR⁴ and R¹¹, OR⁵ and R¹² together form a methylenedioxy group; and
Y is selected from the group consisting of optionally substituted phenyl, optionally substituted benzyl, optionally substituted benzoyl, optionally substituted C₃-C₈ cycloalkyl, optionally substituted CH2—(C₃-C₈ cycloalkyl), optionally substituted 5-6 membered heterocyclyl, and optionally substituted CH2-(5-6 membered heterocyclyl).

134. The composition according to claim 133, wherein the silvestrol analog has the formula; 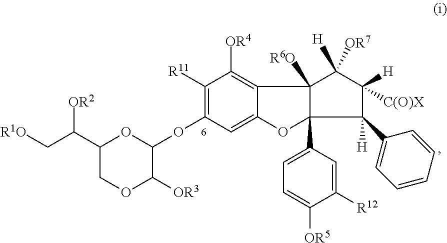
wherein
each R¹-R¹⁰ is independently selected from the group consisting of hydrogen, optionally substituted alkyl, optionally substituted acyl, optionally substituted aryl, optionally substituted arylalkyl, optionally substituted cycloalkylalkyl, optionally substituted arylacyl, optionally substituted cycloalkylacyl and a C-1 linked saccharide;
X is OR⁸ or NR⁹R¹⁰; and
R¹¹ and R¹² are each independently hydrogen or, OR⁴ and R¹¹ or OR⁵ and R¹² together form a methylenedioxy group.

135. A method of vaccinating a subject against cancer, comprising administering to a subject not yet diagnosed with cancer a composition comprising:
a) silvestrol or silvestrol analog; and
b) a vaccine.

136. The method according to claim 135, wherein the subject is vaccinated against a cancer comprising EBV-driven lymphoma, EBV+ lymphomas, nasopharngeal carcinoma, aggressive hematologic malignancies, Hodgkin's lymphomas, Burkitt's lymphoma, or diffuse large B cell lymphoma.

137. The method according to claim 135, wherein the vaccine comprises a preventative vaccine comprising tumor cell vaccines, antigen vaccines, dendritic cell vaccines, DNA vaccines, or vector-based vaccines.

138. The method according to claim 135, wherein the vaccine comprises a therapeutic vaccine comprising tumor cell vaccines, antigen vaccines, dendritic cell vaccines, DNA vaccines, or vector-based vaccines

139. The method according to claim 137, wherein the vaccine comprises an antigen vaccine.

140. The method according to claim 135, wherein the silvestrol analog has the structure: 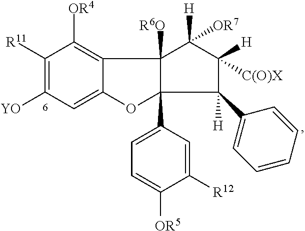
wherein each R⁴-R¹⁰ is independently selected from the group consisting of hydrogen, optionally substituted alkyl, optionally substituted acyl, optionally substituted aryl, optionally substituted arylalkyl, optionally substituted cycloalkylalkyl, optionally substituted arylacyl, optionally substituted cycloalkylacyl and a C-1 linked saccharide;
X is OR⁸ or NR⁹R¹⁰;
R¹¹ and R¹² are each independently hydrogen, or OR⁴ and R¹¹, OR⁵ and R¹² together form a methylenedioxy group; and
Y is selected from the group consisting of optionally substituted phenyl, optionally substituted benzyl, optionally substituted benzoyl, optionally substituted C₃-C₈ cycloalkyl, optionally substituted CH2—(C₃-C₈ cycloalkyl), optionally substituted 5-6 membered heterocyclyl, and optionally substituted CH2-(5-6 membered heterocyclyl).

141. The method according to claim 140, wherein the silvestrol analog has the formula; 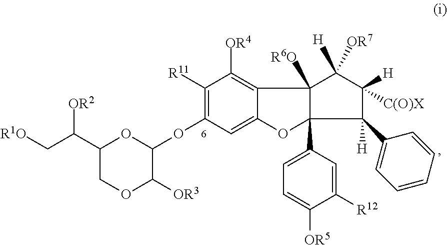
wherein
each R¹-R¹⁰ is independently selected from the group consisting of hydrogen, optionally substituted alkyl, optionally substituted acyl, optionally substituted aryl, optionally substituted arylalkyl, optionally substituted cycloalkylalkyl, optionally substituted arylacyl, optionally substituted cycloalkylacyl and a C-1 linked saccharide;
X is OR⁸ or NR⁹R¹⁰; and
R¹¹ and R¹² are each independently hydrogen or, OR⁴ and R¹¹ or OR⁵ and R¹² together form a methylenedioxy group.

142. The method according to claim 135, wherein the composition is administered in a dosage and frequency synergistically effective to modulate the immune system in the subject, thereby vaccinating the subject against the cancer, wherein humoral immune function in the subject is unaffected, wherein Ig production in the subject is unaffected, wherein IFNg production in the subject remains normal, wherein the composition potentiates virus-specific or tumor-specific cytotoxic T lymphocytes, wherein the composition potentiates virus-specific or tumor-specific T-helper cells, wherein the composition potentiates virus-specific or tumor-specific T-helper cells, or wherein the composition potentiates virus-specific or tumor-specific Th1, Th2, Th17, or Treg cells.

143. A cancer treatment method comprising administering to a patient diagnosed with cancer a composition comprising:
a) silvestrol or silvestrol analog; and
b) a vaccine;
wherein the composition is administered in an amount effective to treat the cancer, and
wherein the cancer comprises EBV-driven lymphoma, EBV+ lymphomas, nasopharngeal carcinoma, aggressive hematologic malignancies, Hodgkin's lymphomas, Burkitt's lymphoma, or diffuse large B cell lymphoma.

144. The method according to claim 143, wherein the vaccine comprises a preventative vaccine comprising tumor cell vaccines, antigen vaccines, dendritic cell vaccines, DNA vaccines, or vector-based vaccines.

145. The method according to claim 143, wherein vaccine comprises a therapeutic vaccine comprising tumor cell vaccines, antigen vaccines, dendritic cell vaccines, DNA vaccines, or vector-based vaccines

146. The method according to claim 145, wherein the vaccine is an antigen vaccine.

147. The method according to claim 143, wherein the silvestrol analog has the structure: 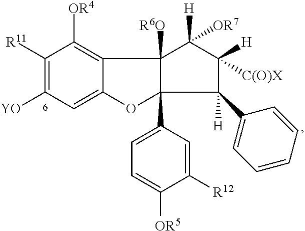
wherein each R⁴-R¹⁰ is independently selected from the group consisting of hydrogen, optionally substituted alkyl, optionally substituted acyl, optionally substituted aryl, optionally substituted arylalkyl, optionally substituted cycloalkylalkyl, optionally substituted arylacyl, optionally substituted cycloalkylacyl and a C-1 linked saccharide;
X is OR⁸ or NR⁹R¹⁰;
R¹¹ and R¹² are each independently hydrogen, or OR⁴ and R¹¹, OR⁵ and R¹² together form a methylenedioxy group; and
Y is selected from the group consisting of optionally substituted phenyl, optionally substituted benzyl, optionally substituted benzoyl, optionally substituted C₃-C₈ cycloalkyl, optionally substituted CH2—(C₃-C₈ cycloalkyl), optionally substituted 5-6 membered heterocyclyl, and optionally substituted CH2-(5-6 membered heterocyclyl).

148. The method according to claim 147, wherein the silvestrol analog has the formula; 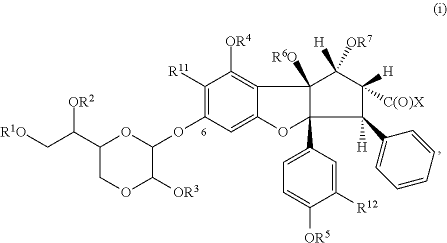
wherein
and each R¹-R¹⁰ is independently selected from the group consisting of hydrogen, optionally substituted alkyl, optionally substituted acyl, optionally substituted aryl, optionally substituted arylalkyl, optionally substituted cycloalkylalkyl, optionally substituted arylacyl, optionally substituted cycloalkylacyl and a C-1 linked saccharide;
X is OR⁸ or NR⁹R¹⁰; and
R¹¹ and R¹² are each independently hydrogen or, OR⁴ and R¹¹ or OR⁵ and R¹² together form a methylenedioxy group.
